SEQUENCE LISTING (20 sequences)

SEQ ID NO: 1 - DNA, 14825 bp, HIV-1
  source 1..14825: mol_type = genomic DNA
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacccaggaggtagaggttgcagtgagccaagatcgcgccactgcattccagcctgggcaagaaaacaagactgtctaaaataataataataagttaagggtattaaatatatttatacatggaggtcataaaaatatatatatttgggctgggcgcagtggctcacacctgcgcccggccctttgggaggccgaggcaggtggatcacctgagtttgggagttccagaccagcctgaccaacatggagaaaccccttctctgtgtatttttagtagattttattttatgtgtattttattcacaggtatttctggaaaactgaaactgtttttcctctactctgataccacaagaatcatcagcacagaggaagacttctgtgatcaaatgtggtgggagagggaggttttcaccagcacatgagcagtcagttctgccgcagactcggcgggtgtccttcggttcagttccaacaccgcctgcctggagagaggtcagaccacagggtgagggctcagtccccaagacataaacacccaagacataaacacccaacaggtccaccccgcctgctgcccaggcagagccgattcaccaagacgggaattaggatagagaaagagtaagtcacacagagccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccaggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcaggcctccacctgggtcttggaatgtgtcccccgaggggtgatgactacctcagttggatctccacaggtcacagtgacacaagataaccaagacacctcccaaggctaccacaatgggccgccctccacgtgcacatggccggaggaactgccatgtcggaggtgcaagcacacctgcgcatcagagtccttggtgtggagggagggaccagcgcagcttccagccatccacctgatgaacagaacctagggaaagccccagttctacttacaccaggaaaggc

SEQ ID NO: 2 - protein, 192 aa, HIV-1
MENRWQVMIVWQVDRMRINTWKRLVKHHMYISRKAKDWFYRHHYESTNPKISSEVHIPLGDAKLVITTYWGLHTGERDWHLGQGVSIEWRKKRYSTQVDPDLADQLIHLHYFDCFSESAIRNTILGRIVSPRCEYQAGHNKVGSLQYLALAALIKPKQIKPPLPSVRKLTEDRWNKPQKTKGHRGSHTMNGH

SEQ ID NO: 3 - protein, 147 aa, HIV-1 [sequence omitted]

SEQ ID NO: 4 - protein, 86 aa, HIV-1
MEPVDPRLEPWKHPGSQPKTACTNCYCKKCCFHCQVCFMTKALGISYGRKKRRQRRRAHQNSQTHQASLSKQPTSQSRGDPTGPKE

SEQ ID NO: 5 - protein, 854 aa, HIV-1
MRVKEKYQHLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGAASMTLTVQARQLLSDIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 6 - DNA, 645 bp, Rhesus macaque
  source 1..645: mol_type = genomic DNA
atggaggaggaaaagaggtggatagcagttcccacatggaggataccggagaggctagagaggtggcatagcctcataaaatatctgaaatataaaactaaagatctacaaaaggtttgctatgtgccccattttaaggtcggatgggcatggtggacctgcagcagagtaatcttccccctacaggaaggaagccatttagaagtacaagggtattggcatttgacaccagaaagagggtggctcagtacttatgcagtgaggataacctggtactcaaggaacttttggacagatgtaacaccagactatgcagacattttactgcatagcacttatttcccttgctttacagcgggagaagtgagaagggccatcaggggagaacaactgctgtcttgctgcaagttcccgagagctcataggtaccaggtaccaagcctacagtacttagcactaaaagtagtaagcgacgtcagatcccagggagagaatcccacctggaaacagtggagaagagacaataggagaggccttcgaatggctaaacagaacagtagaggagataaacagagaggcagtaaaccacctaccaagggagctgattttccaggtttggcaaaggtcttgggaatactggcatga

SEQ ID NO: 7 - DNA, 645 bp, Pig-tail macaque
  source 1..645: mol_type = genomic DNA
atggaggaggaaaagaggtggatagcagttcccacatggaggataccggagaggctagagaggtggcatagcctcataaaatatctgaaatataaaactaaagatctacaaaaggtttgctatgtgccccatcataaggtcggatgggcatggtggacctgcagcagagtaatcttcccactacaagaagaaagccagttagaagtacaagggtattggaatttaacaccagaaagagggtggctcagtacttatgcagtgagaataacctggtactcaaggaacttttggacagatgtaacaccagactatgcagacattttactgcatagcacttatttcccttgctttacagcgggagaagtgagaagggccatcaggggagaacaactgctgtcttgctgcaggttcccgagagctcataagaaccaggtaccaagtctacagtacttagcactgagagtagtaagttacgtcagatcccagagagagaatcccacctggaaacagtggagaagagacaataggagaagccttcgaatggctaaacagaacagtagaggagataaacagagaggcagtaaaccacctaccaagggagctgattttccaggtttggcaaaggtcttgggaatactggcatga

SEQ ID NO: 8 - protein, 192 aa, HIV-1; identical to SEQ ID NO: 2

SEQ ID NO: 9 - protein, 146 aa, HIV-1
PIVQNLQGQMVHQAISPRTLNAWVKVVEEKAFSPEVIPMFSALSEGATPQDLNTMLNTVGGHQAAMQMLKETINEEAAEWDRLHPVPGPIPAGQMREPRGSDIAGTTSTLQEQIGWMTHNPPIPVGEIYKRWIILGLNKIVRMYSP

SEQ ID NO: 10 - protein, 146 aa, HIV-1
PIVQNLQGQMVHQAISPRTLNAWVKVVEEKAFSPEVIPMFSALSEGATPQDLNTMLNTVGGHQAAMQMLKETINEEAAEWDRLHPVPGPIPAGQMREPRGSDIAGTTSTLQEQIGWMTRNPPIPVGEIYKRWIILGLNKIVRMYSP

SEQ ID NO: 11 - protein, 146 aa, HIV-1; identical to SEQ ID NO: 10

SEQ ID NO: 12 - protein, 86 aa, HIV-1
MEPVDPRLEPWKHPGSQPKTACTNCYCKKCCFHCQVCFMTKALGISYGRKKRRQRRRTHQNSQTHQASLSKQPTSQSRGDPTGPKE

SEQ ID NO: 13 - protein, 854 aa, HIV-1
MRVKEKYQRLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGAASMTLTVQARQLLSDIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 14 - protein, 854 aa, HIV-1
MRVKEKYQHWWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGAASMTLTVQARQLLSDIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 15 - protein, 854 aa, HIV-1
MRVKEKYQHLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 16 - protein, 854 aa, HIV-1
MRVKEKYQHWWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 17 - protein, 854 aa, HIV-1
MRVKEKYQRLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 18 - protein, 854 aa, HIV-1
MRVKEKYQRWWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 19 - protein, 854 aa, HIV-1
MRVKEKYQHLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGAASMTLTVQARQLLSDIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 20 - protein, 854 aa, HIV-1
MRVKEKYQRWWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

(19) United States
(12) Patent Application Publication
(10) Pub. No.: US 2025/0304923 A1
(43) Pub. Date: Oct. 2, 2025

(54) Novel HIV-1 Variants And Their Methods Of Use In An Animal Challenge Model

(71) Applicants: The Regents of the University of Colorado, a body corporate, Denver, CO (US); Board of Regents, The University of Texas System, Austin, TX (US)

(72) Inventors: Sara L. Sawyer, Boulder, CO (US); Nicholas Meyerson, Broomfield, CO (US); Greg Wilkerson, New Hill, NC (US); Vanessa Lamae BAUER, Longmont, CO (US); Will Tyler Fattor, Black Hawk, CO (US)

(21) Appl. No.: 19/230,977

(22) Filed: Jun. 6, 2025

Related U.S. Application Data
(63) Continuation-in-part of application No. PCT/US23/82939, filed on Dec. 7, 2023.
(60) Provisional application No. 63/431,003, filed on Dec. 7, 2022.
(60) Provisional application No. 63/522,321, filed on Jun. 21, 2023.

(51) Int. Cl. C12N 7/00

(52) U.S. Cl. CPC C12N 7/00; C12N 2740/16021; C12N 2740/16045

(57) ABSTRACT

Genetically modified HIV-1 variants capable of infecting owl monkeys that includes one or more novel point mutations coupled with the replacement of the viral infectivity factor (Vif) gene.

### CROSS-REFERENCE TO RELATED APPLICATIONS

This application is a bypass continuation-in-part of PCT Application No. PCT/US2023/082939 having an international filing date of Dec. 7, 2023, which designated the United States, which PCT application claims the benefit of and priority to U.S. Provisional Application No. 63/431,003, filed Dec. 7, 2022, and U.S. Provisional Application No. 63/522,321, filed Jun. 21, 2023, the specification, claims and drawings of which are incorporated herein by reference in their entirety.

### GOVERNMENT INTEREST

This invention was made with Government support under grant number DPI-DA-046108 awarded by the National Institutes of Health (NIH). The U.S. Government has certain rights in this invention.

### SEQUENCE LISTING

The instant application contains contents of the electronic sequence listing (90245-00904-Sequence-Listing.xml; Size: 42,944 bytes; and Date of Creation: Jun. 5, 2025) is herein incorporated by reference in its entirety.

### DEPOSIT INFORMATION

The HIV viral strains ATCC Patent Deposit No. PTA-127572 (HIVom v1.1), ATCC Patent Deposit No. PTA-127573 (HIVom v1.2), ATCC Patent Deposit No. PTA-127574 (HIVom v1.3), ATCC Patent Deposit No. PTA-127575 (HIVom v1.4), have been deposited in an international depository under conditions that assure that access to the culture will be available during the pendency of this patent application and any patent(s) issuing therefrom to one determined by the Commissioner of Patents and Trademarks to be entitled thereto under 37 C.F.R. 1.14 and 35 U.S.C. 122. These strains have been deposited in the American Type Culture Collection (ATCC), at 10801 University Boulevard, Manassas, VA., 20110-2209 United States of America.

### NAMING CONVENTIONS

The HIV viral strains of the present disclosure are identified herein by ATCC Deposit Number, designated ATCC Name, as well as alternative naming conventions which are all used interchangeably herein, and summarized in the Table below:

| ATCC PATENT | | ALTERNATIVE |
|---|---|---|
| DEPOSIT NO. | ATCC NAME | NAMES |
| PTA-127572 | HIVom v1.1 | HIVom1 |
| | | HIVom vl.1 |
| | | NLRM |
| | | HIVnom2 |
| PTA-127573 | HIVom v1.2 | HIVom1* |
| | | HIVom vl.2 |
| | | NLRM + 4 |
| | | HIVnom3 |
| PTA-127574 | HIVom v1.3 | HIVom2 |
| | | HIVom vl.3 |
| | | NLRP |
| | | HIVnom1 |
| PTA-127575 | HIVom v1.4 | HIVom2* |
| | | HIVom vl.4 |
| | | NLRP+ |

### TECHNICAL FIELD

The present invention is generally directed to the production and use of novel genetically modified Human Immunodeficiency Virus-1 (HIV-1) variants, and specifically their use in an animal challenge model. In one preferred embodiment, the HIV-1 variants of the invention can be used in a primate challenge model for the development of vaccines and other therapeutics for the same.

### BACKGROUND

Over the past 40 years, thousands of papers have been published on macaque and rodent models of HIV-1/AIDS. The progress made in these models is staggering, particularly in understanding virus pathology, immunology, and the development of antiviral medications that are now routinely used worldwide. Animal models are also essential for the trial-and-error development of vaccines, although vaccine efforts have not yet been successful. Vaccine design is difficult and requires the pairing and optimization of multiple factors: the immunogenic payload, the delivery system (e.g., subunit, mRNA, viral vector), the adjuvant, and the dosing regimen. In theory, animal models enable tuning of each of these parameters until they are effective, facilitating more and better vaccine candidates advancing to trials in humans. Applicants have developed a model which reliably results in HIV-1 replication in fully immunocompetent monkeys. As described herein, Applicants have achieved successful infection of Nancy Ma's owl monkeys (Aotus nancymaae) with an HIV-1 that is 93% wildtype in genomic sequence. Because the virus is a minimally modified form of HIV-1 it contains most of the epitopes relevant to humoral and cell-mediated immunity. Infected owl monkeys recapitulate key aspects of human HIV-1 infection: an acute phase of viral replication with peak plasma viremias up to 107 HIV-1 genomes per milliliter, subsequent reduction in viremia to a set point value, seroconversion, and establishment of a persistent virus reservoir in cells in lymph nodes and possibly other locations.

Owl monkeys represent a new pre-clinical model for the trial-and-error refinement of HIV-1 vaccines. Experimental vaccine formulations could be administered to the owl monkey, the owl monkey's immune system would be allowed to respond, and then the efficacy of the vaccine could be tested by infecting the animals with the viruses described herein.

### SUMMARY OF THE INVENTION

The present invention describes an immune-competent animal model for HIV-1. In a preferred embodiment, the invention include an immune-competent animal model for HIV-1 using owl monkeys. These animals are particularly useful as they demonstrate all key landmarks of HIV-1 infection seen in humans: acute infection, seroconversion, lymphocyte responses, and establishment of the latent reservoir. In one aspect, owl monkeys can serve as model for studying HIV-1 transmission, immunity, and control. In one aspect, the present invention includes genetically modified HIV-1 variants that are capable of infecting owl monkeys. In one preferred aspect, owl monkeys are infected with an HIV-1 variant of the invention that is substantially wildtype. In one preferred aspect, an HIV-1 variant of the invention includes a plurality of novel point mutations coupled with the replacement of the viral infectivity factor (Vif) gene.

The present invention includes HIV-1 variants configured to infect owl monkeys, which can be selected from the strains:
ATCC Patent Deposit No. PTA-127572
ATCC Patent Deposit No. PTA-127573
ATCC Patent Deposit No. PTA-127574
ATCC Patent Deposit No. PTA-127575)
(See Naming Conventions Section for ATCC Name and Alternative Name)

In one aspect, the invention includes a HIV-1 variant genetically modified to infect Aotus nancymaae (owl monkey), wherein said HIV-1 variant includes a genetically modified capsid peptide according to SEQ ID NO. 3, or a fragment or variant thereof, having the following mutations: a ΔH87 mutation; an A88P mutation; an A92P mutation; a P93A mutation.

In a preferred embodiment, the HIV-1 variant is further genetically modified such that the endogenous Viral infectivity factor (Vif) is disrupted or replaced with a simian immunodeficiency virus Viral infectivity factor (SIVVif), which can be selected from: SIVmac (SEQ ID NO. 6), SIVptm (SEQ ID NO. 7), or a fragment or variant thereof. In this preferred aspect, the SIVVif is inserted upstream of a Pol region and downstream of a Vpr region.

In one aspect, the invention includes a HIV-1 variant genetically modified to infect Aotus nancymaae (owl monkey), wherein said HIV-1 variant includes one or more additional mutations to the Capsid, Tat or Env peptides, where in the mutations are selected from: an arginine substituted at position 120 of the HIV-1 Capsid protein; a threonine substituted at position 58 of a HIV-1 Tat protein; an arginine substituted at position 9 of a HIV-1 Env protein; a tryptophan at position 10 of the HIV-1 Env protein; and a glycine substituted at position 545 of the HIV-1 Env protein; or a glycine substituted at position 167 of the HIV-1 Env protein; or a combination of the same.

Additional aspects of the invention include an owl monkey infected in vivo, ex vivo, or in vitro with one or more of the HIV-1 variants described herein.

Additional aspects of the invention will become apparent based on the specification, drawing and claims provided below.

### BRIEF DESCRIPTION OF THE FIGURES

FIG. 1A-F. Determining immune bypasses required for host switching of HIV-1 into owl monkeys. a, Restriction of HIV-1 by owl monkey TRIMCyp variants. CRFK cells stably expressing 14 TRIMCyp variants were exposed to VSIV G-pseudotyped HIV-1-GFP particles. The percentage of cells expressing GFP was normalized relative to cells not expressing any TRIMCyp. Experiments were performed in two biological replicates, each with two technical replicates; error bars represent the standard error of the mean (SEM). b, Screen for mutations in HIV-1 that result in escape from owl monkey TRIMCyp. CRFK cells stably expressing one of two TRIMCyp variants were transduced with an increasing volume of VSIV G-pseudotyped HIV-1-GFP particles containing cyclophilin A-binding loop amino-acid changes (G89V^{22,23} or V86A²⁴) or loops from diverse lentiviruses (full set tested shown in FIG. S5) as indicated at the top. c, Alignment of the cyclophilin A-binding loops of HIV-1 (GenBank #AF324493) and SIVrcm (GenBank #AF028608). d, HIV-1 replication on owl monkey cells. Hut78 (human) and OMK (owl monkey) cells, both expressing human CD4 and CCR5, were exposed to HIV-1 with or without four mutations re-constituting the cyclophilin A-binding loop (CBL) from SIVrem as in (c) at an MOI=0.1. Growth curves were generated by measuring the concentration of HIV-1 p24 (CA) in supernatants at the indicated time points. Note that OMK cells derive from an owl monkey of a different species 21, but were used here because there are no immortalized lines from Nancy Ma's owl monkeys. e, Restriction of HIV-1 by owl monkey APOBEC3G variants. 293T cells were transfected with plasmids containing HIV-1, HIV-1 Δvif, or HIV-1 encoding vif from SIVmac or SIVmne, together with plasmids encoding either human or the indicated owl monkey APOBEC3G variants. Infectious virus produced in each condition was measured by adding viral supernatants to reporter (firefly luciferase) TZM-bl cells. Light units were normalized to cells not expressing APOBEC3G. Two biological replicates are shown, each with four technical replicates. Error bars represent the SEM. f, Genome schematics of the engineered HIV-1 strains HIVnom1 and HIVnom2.

FIG. 2A-C. HIV infection of owl monkeys. a, Owl monkeys CA (5-year-old male) and SO (15-year-old female) were injected intravenously with HIVnom1, and KI (15-year-old male) and RI (16-year-old male) with HIVnom2. Plasma viremia over time was measured by RT-qPCR. The “0” timepoint represents blood samples collected from the leg opposite to the site of virus injection five minutes after inoculation. Dotted horizontal lines depict the limit of quantification of 80 HIV-1 genomes per milliliter. Lack of detection of any HIV-1 genomes is graphed as non-detected (ND). Stars indicate the weeks when animals seroconverted. b, Frozen plasma collected from monkey RI was transfused into owl monkey AB (13-year-old female). Virus from animal AB, which had acquired four point mutations, was renamed HIVnom3. Frozen plasma and fresh whole blood collected from monkey AB were mixed and transfused into two additional owl monkeys, BO (4-year-old male) and ON (21-year-old male). c, Genome schematic of HIVnom3.

FIG. 3A-C. HIVnom3 is an owl monkey-adapted virus. a, Owl monkeys ME (5-year-old male), SI (5-year-old female), ST (13-year-old male), and JI (13-year-old female) were injected intravenously with a 50:50 mix of HIVnom2 and HIVnom3 (9×10⁵ infectious units of each). SI, ST, and JI were treated with dexamethasone during the indicated time periods. Plasma viremia over time was measured by RT-qPCR. The “0” timepoint represents blood samples collected from the leg opposite to the site of virus injection five minutes after inoculation. The dotted horizontal line is the assay limit of quantification of 80 HIV-1 genomes per mL, “ND” means no genomes detected. Stars indicate the weeks when animals scroconverted; horizontal bars indicate average plasma viremia over the indicated time span. b, Sanger sequencing plots of virus from animal JI, with plots focused around the four mutational sites specific to HIVnom3. Nucleotide numbers are based on the first base in the 5′ LTR of HIV-1 (NL4-3 GenBank #AF324493.2). Full data for all animals are shown in FIGS. S15-S18. c, Weeks to seroconversion is shown for all animals in this study, separated by whether they were infected by the original viruses (HIVnom1 or HIVnom2) or the adapted virus (HIVnom3). Lines depict median values. Animals RI and AB are indicated because these are the two animals in which virus adaption occurred during in vivo serial passage (see FIG. S9).

FIG. 4A-I. Immunological and histological responses to infection in owl monkeys. a, Presence of antibodies against three HIV-1 proteins in plasma of HIVnom-infected animals (i.e. seroconversion), determined by the Bio-Rad Genius HIV1/2 system (Life Science). b, Detection of neutralizing antibodies in plasma of the indicated animals. Owl monkey plasma from different weeks post-exposure was serially diluted (X-axis) and then pre-mixed with HIVnom3. TZM-bl cells were then exposed to this mixture, and virus entry quantified by luciferase assay (Y-axis), normalized to cells infected with virus not pre-mixed with plasma (first point on X-axis). c-i, RNAscope RNA in situ hybridization assay performed on necropsy-derived tissues. The probes in this assay anneal to HIV-1 transcribed RNA. Animals with asterisks next to names were infected but nonviremic at time of necropsy. c,e-i, Representative micrographs of formalin-fixed tissues stained with RNAscope probes. Positive results are represented by brown staining and emphasized with red arrows. Magnification 60×. d, A summary of RNAscope studies performed. ND, assay not done.

FIG. S1A-D. Population genetics of genes encoding the CD4 receptor and three major lentivirus restriction factors in a captive colony of Nancy Ma's owl monkeys. Genes encoding CD4, Tetherin, TRIMCyp, and APOBEC3G were sequenced and SNP linkages (i.e. alleles) were derived for 191 captive Nancy Ma's owl monkeys (diploid=382 alleles determined for each gene). The number of alleles identified for each gene is shown by circles. The size of each circle indicates the number of times that each allele was observed. Tick marks between circles indicate the amino acid position(s) in the deduced protein sequences in which nonsynonymous SNPs differentiate each pair of alleles. Alleles were numbered in the order that they were discovered. The graphics were generated using PopART69. a, 18 different CD4 alleles were identified in this colony, and allele 1 appeared 101 times. b, 8 different Tetherin alleles were identified, and allele 4 appeared 104 times. c, 37 different TRIMCyp alleles were identified, and no allele was found more than 27 times. d, 14 different APOBEC3G alleles were identified, and allele 1 appeared 185 times.

FIG. S2. Nancy Ma's owl monkey CD4 variants enable HIV-1 entry. Pseudotyped HIV-1-EGFP was produced by co-transfecting HEK 293T cells with plasmids containing a ΔEnv EGFP-encoding HIV-1 genome (NL4-3ΔEnv-EGFP) and an HIV-1 Env (isolate USI.EC6, GenBank #MK501602). Pseudotyped virions were concentrated from supernatant. Cf2Th cells were transduced to stably produce human C-C motif chemokine receptor 5 (CCR5) alone or together with human CD4 or various Nancy Ma's owl monkey CD4 variants (FIG. S1). These Cf2Th cell lines were plated and 24 h later were exposed to an equivalent volume of virions. 48 h later, cells were harvested and assayed for the EGFP signal by flow cytometry. Values were normalized to the average signal on positive control cells (complemented with human CD4) and expressed as percentage of the signal from that cell line. The limit of detection (dashed line) was set based on the false positive signal observed after the conservative gating of 1% of uninfected cells. Data points represent six individual technical replicates, three each from two independent experiments.

FIG. S3A-C. Nancy Ma's owl monkey Tetherin does not, or only weakly, restricts HIV-1. a, Western blot showing the expression of various Tetherin variants internally tagged with hemagglutinin (Tetherin-HAs) with variants 1-8 representing eight unique Nancy Ma's owl monkey Tetherins (FIG. S1). 293T cells in 24-well dishes were transfected with 50 ng of each Tetherin-HA-encoding plasmid. Whole-cell extracts (WCE) were harvested 48 h later and subjected to western blotting using Anti-HA-Peroxidase antibody (1:5,000 dilution, Roche #12013819001). Non-glycosylated forms of Tetherin-HA are approximately 25 kDa. b, 50 ng of the indicated Tetherin-HA plasmids were co-transfected into 293T cells with 300 ng of a plasmid containing full-length HIV-1 NL4-3 wildtype or NL4-3Δvpu provirus. Forty-eight hours post-transfection, virions in clarified supernatants were enumerated by titration onto TZM-bl reporter cells using a firefly luciferase readout (RLUs, relative light units). Values were normalized to a sample that did not contain Tetherin. Shown are the means of two biological replicates, each performed in triplicate. Error bars represent standard errors of the means (SEMs). c, For each of the samples in (b), whole cell extracts and crude virion preparations were probed to detect HIV-1 capsid by western blot using an anti-p24 (CA) antibody (1:1,000, BEI #ARP-3537). Endogenous actin beta was detected using Anti-β-Actin antibody (C4) (1:1,000, Santa Cruz Animal Health #sc-47778) as a loading control. Numbers at the bottom of each blot are the normalized band intensity values of the anti-p24 (CA) signal, normalized using the no-Tetherin control, done using Image J. Note the bottom row of the blot, which is key because it shows virions released into the supernatant.

FIG. S4A-D. Single-cycle titration of engineered viruses. Plasmids encoding full-length HIV-1 NL4-3 proviruses were transfected into 293T cells and virus titers were determined using (a,b) the TZM-bl β-gal assay to measure infectious units (i.u.) or (c) the SG-PERT assay to measure reverse transcriptase (RT) activity. d, Infectious units per picogram of reverse transcriptase were calculated as proxies for overall fitness of each virus. Our engineered viruses lose approximately one order of magnitude in fitness compared to NL4-3 (most apparent in panels b,d), whereas the G89V mutation (panel a) causes the virus to lose two orders of magnitude in fitness. Not shown in this experiment: Applicants were unable to obtain significant spreading infection with viruses containing the G89V mutation.

FIG. S5A-B. Natural variation in HIV-1 and SIV capsid cyclophilin A-binding loops. a, Amino-acid alignment of the cyclophilin A-binding loops of various HIV-1 and SIV strains. These loops were each introduced into HIV-1 and tested for the ability to bypass Nancy Ma's owl monkey TRIMCyp restriction. Numbers at the top indicate amino-acid residue positions relative to the start of HIV-1 NL4-3 p24 (CA). The SIVrem-GAB variant was the source of the loop that yielded escape from owl monkey TRIM-Cyp. b, A WebLogo alignment of the cyclophilin A-binding loops of four HIV-1 subtypes. Sequences were obtained from the Los Alamos HIV-1 sequence database. Variable sites are highlighted in yellow.

FIG. S6A-B. Screening of 42 SIV and HIV Vifs to evaluate their ability to degrade Nancy Ma's owl monkey APOBEC3G. Western blots in which each lane represents an experiment based on co-transfection of two plasmids into 293T cells: one expressing an HA-tagged APOBEC3G variant and one expressing an HA-tagged Vif from the indicated HIV-1, SIV, or no Vif. Actin beta was used as a loading control. Empty plasmids served as controls. a, Asterisks indicate Vifs effective at degrading owl monkey APOBEC3G. b, Effective Vifs identified in (a) were then tested against the four Nancy Ma's owl monkey APOBEC3G variants encoded by the four major alleles identified in FIG. S1.

FIG. S7A-B. Some SIV Vifs neutralize owl monkey APOBEC3G proteins. 293T cells were co-transfected with 300 ng of plasmid containing a full-length HIV-1 (Q23-17⁶²) or HIV-1 (Q23-17Δvif; FIG. S24) provirus, 200 ng of plasmid encoding HA-tagged HIV-1 or SIV Vif, and 25 ng of plasmid encoding HA-tagged human APOBEC3G or 200 ng encoding Nancy Ma's owl monkey APOBEC3G. a, Forty-eight hours post-transfection, cells were collected and lysed, and lysate was subjected to western blotting with anti-HA antibodies to detect Vif mediated degradation of APOBEC3G. Actin beta was used as a loading control. Shown data are representative of two independent experiments. b, Forty-eight hours post-transfection, supernatants were collected and viruses were enumerated. Equivalent volumes of each supernatant were added to reporter (firefly luciferase) TZM-bl cells. Forty-eight hours later, the TZM-bl cells were lysed and assayed for luciferase relative light units (RLUs). All results are background-subtracted and normalized to no-APOBEC3G control plasmid. Error bars represent the standard errors of the mean (SEM) from two biological replicates each with four technical replicates.

FIG. S8. Owl monkey immune cell dynamics during dexamethasone administration. In the top graph, the plasma viremia is reported for each of three animals over the course of the experiment. ND=no HIV-1 RNA detected. In the remainder of the rows, flow cytometry was performed on fresh Nancy Ma's owl monkey blood to enumerate the indicated cell types over the time course of infection. Antibodies: CD8 Invitrogen #MHCD0801; CD4 BD #550631; CD3 BD #563916 or #563918; CD20 BD #340941; CD16 BD #563892; CD14 BD #557923.

FIG. S9. Mutations acquired in the HIVnom2 genome during serial passage. HIVnom2 proviruses were Sanger-sequenced from peripheral blood mononuclear cells (PBMCs) collected at various timepoints after exposure for three animals in the serial transmission chain. All fixed DNA mutations observed are listed. In green font, and boxed in green, are the four non-synonymous mutations acquired by HIVnom2 in its evolution to HIVnom3. Below the black line are two fixed mutations that arose in animal RI, but after the week 22 transfusion to animal AB. LTR, long terminal repeat; ND, not determined; wk, week. Numbering is based on wildtype HIV-1 (NL4-3).

FIG. S10A-E. p24 (CA) H120R. a, Logos plot made for a partial region of HIV-1 (NL4-3) wildtype p24 (CA), using sequences from the “Subtype reference” (four per subtype, no recombinants, group M only) in the Los Alamos National Lab HIV Sequence Database. Position 120 (red arrow) is never occupied by arginine in any reference subtype, but arginine arose in this position in a Nancy Ma's owl monkey. b, Alignment of SIVcpz and SIVgor strains in this region, with p24 (CA) position 120 highlighted in yellow. Arginine occupies this position, or the position just before in a gapped region (orange), in most SIVgor strains analyzed here. c-e, Co-crystal structures⁷¹⁻⁷³ are shown for monomeric HIV-1 p24 (CA) (tan) in complex with the cyclophilin domains of three host binding partners (purple). In p24 (CA), position 120 is indicated in green and the cyclophilin A-binding loop in red. Amino acid differences between the host binding partners and their Nancy Ma's owl monkey orthologs are indicated by purple stars.

FIG. S11A-B. Tat A58T. a, Partial alignment of HIV-1 Tat, from wildtype NL4-3 (top), the in vivo adapted HIVnom3 virus (second row), and representative transmitted/founder viruses (referenced with their GenBank accession numbers). b, Logos plot of the region around the Tat A58T change, made using sequences in panel a. Logos plot was made using WebLogo Of note, A58T increases activation of gene transcription from the HIV-1 long terminal repeat⁷⁴.

FIG. S12A-B. gp160 H9R and L10W. a, Partial alignment of HIV-1 gp160, showing NL4-3 wildtype (top), the viruses isolated after passage in monkeys AB and RI (second and third rows), and representative transmitted/founder viruses (referenced with their GenBank accession numbers). b, Logos plot of the region around the H9R/L10W changes, made using sequences in panel a. Logos plot was made using WebLogo.

FIG. S13. gp160 D545G. Partial alignment of HIV-1 gp160 showing wildtype NL4-3 (top), the in vivo adapted HIVnom3 (second row), and representative transmitted/founder viruses (referenced with their GenBank accession numbers).

FIG. S14. Adaptation of HIVnom to owl monkeys does not significantly affect fitness in human cells. Human SUP-T1 T-lymphoblasts were exposed to HIVnom2 or HIVnom3 and viruses were quantified over 10 days by measuring reverse transcriptase (RT) in the media. The mock samples show the amount of reverse transcriptase detected in media collected from uninfected cells plated at the same time, over the same time course.

FIG. S15. Animal ME. Forward and reverse sequencing traces are shown across the four point-mutation sites characterizing HIVnom3. At week 0, ME was inoculated with a 50:50 mix of HIVnom2 and HIVnom3. Five minutes after inoculation, plasma was collected and viruses therein sequenced. At later weeks, DNA was purified from collected peripheral blood mononuclear cells (PBMCs) to sequence proviral DNA. SNP, single nucleotide polymorphism.

FIG. S16. Animal SI. Same experiment as described in FIG. S15 for animal SI.

FIG. S17. Animal ST. Same experiment as described in FIG. S15 for animal ST.

FIG. S18. Animal JI. Same experiment as described in FIG. S15 for animal JI.

FIG. S19. Time of seroconversion is unrelated to infectious dose. Each icon represents a different infected monkey in this study. The time to seroconversion for that individual is plotted on the Y axis, and the plasma viremia at 5 minutes after inoculation with HIV are shown on the X axis. Triangles, circles, and squares represent the virus to which each animal was exposed. Note that animal AB received virus via transfusion from animal RI, and the virus in this transfusion only had 3 of the 4 adaptive mutations that eventually were fixed in HIVnom3 (FIG. S9).

FIG. S20. Neutralization of HIVnom3 by common HIV-1 neutralizing monoclonal antibodies. Monoclonal antibodies were mixed with a 1:10 dilution of heat-inactivated owl monkey plasma and 4000 IU of HIVnom3. Concentrations on the x-axis are expressed in micrograms per milliliter (ug/mL) and the antibody cocktail consisted of all 4 antibodies at equal concentration (adding up to 10 μg/mL final concentration). The samples were then serially 2-fold diluted and incubated for 1 h at 37C to allow antibody binding to occur. Following the 1 h incubation, TZM-bl cell culture media (cells plated the day before at 1.1×104 cells/well of a 96-well plate) was removed and replaced with virus+plasma-antibody mixture. The samples were allowed to incubate for 48 h after which the cells were lysed and relative light units were measured using a luminometer. Light units were normalized to those from cells exposed to an equal amount of virus lacking the plasma-antibody mixture and plotted as percent infectivity. Uninfected cells were used to correct for background luciferase activity. Antibodies are: VRC01 (BEI #ARP-12033), NIH45-46 (BEI #ARP-12174), 10E8 (BEI #ARP-12294), 3BNC117 (BEI #ARP-12474).

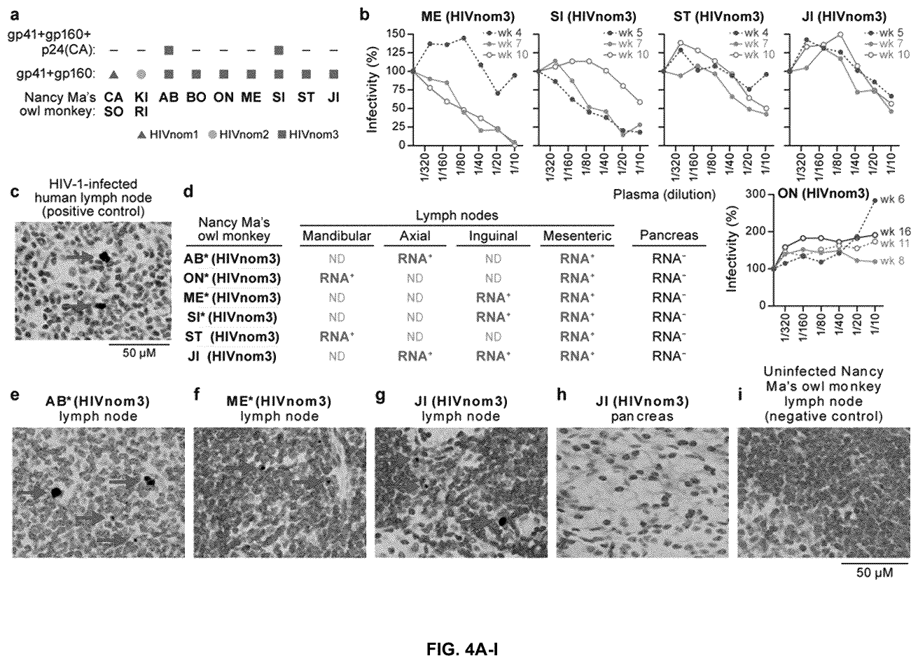

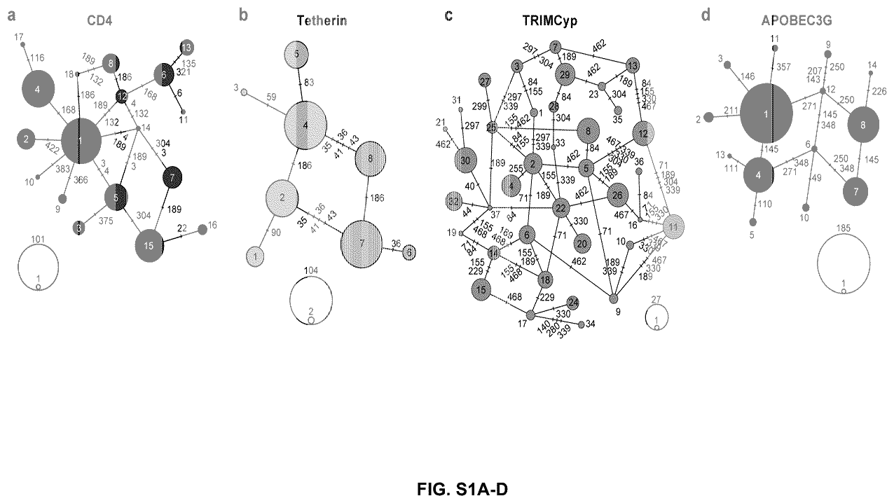

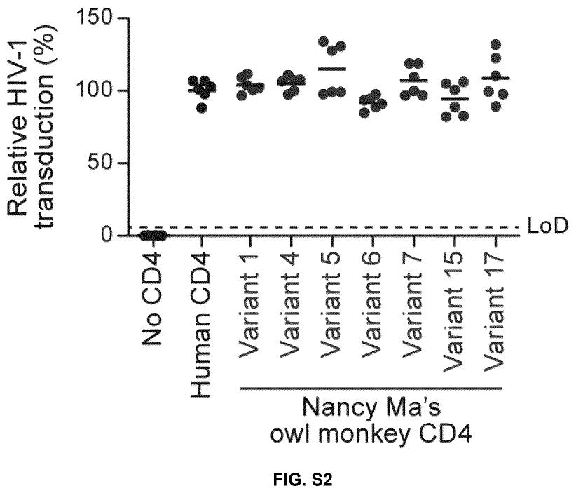

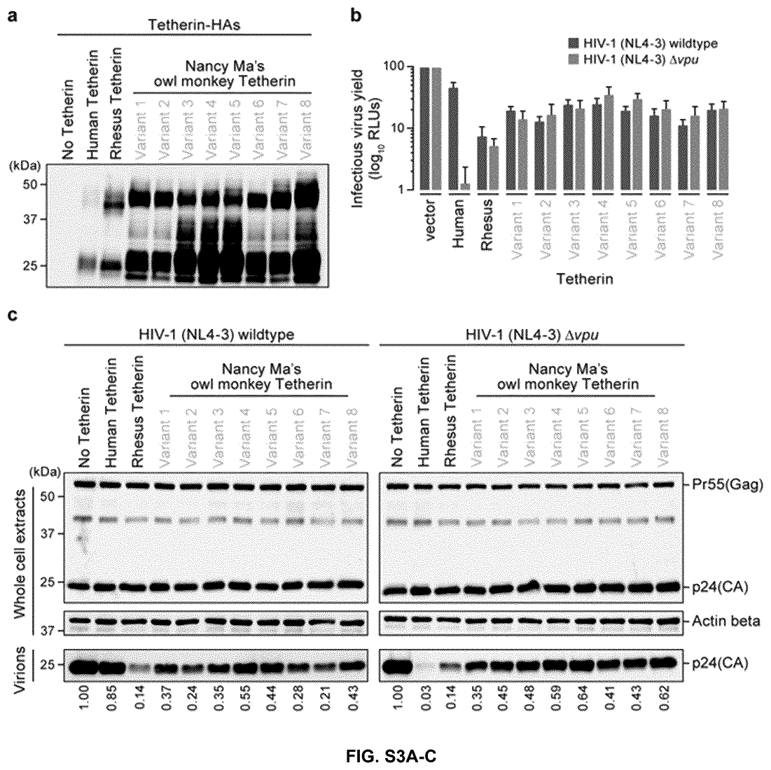

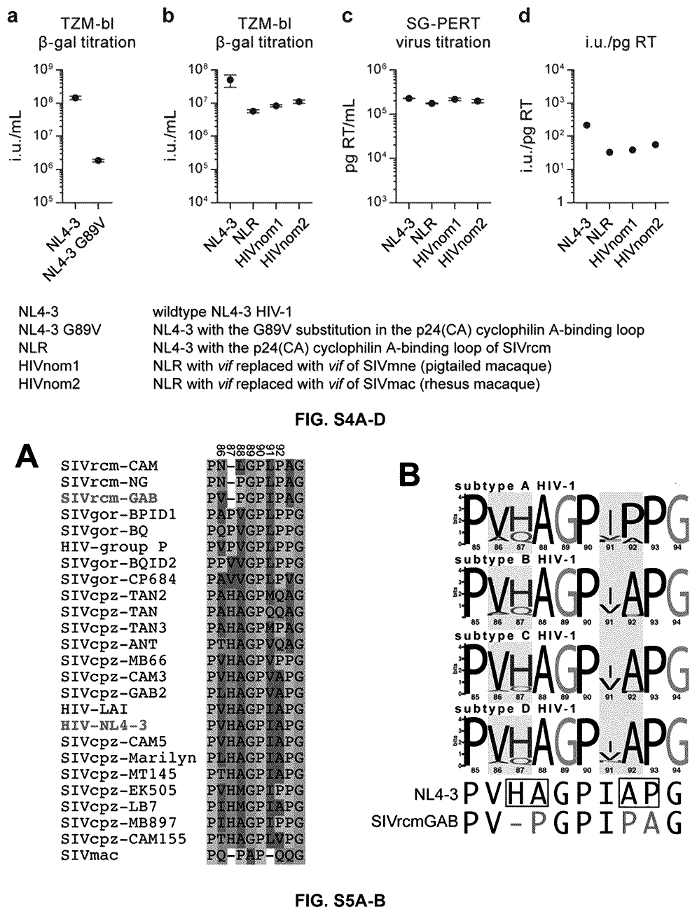

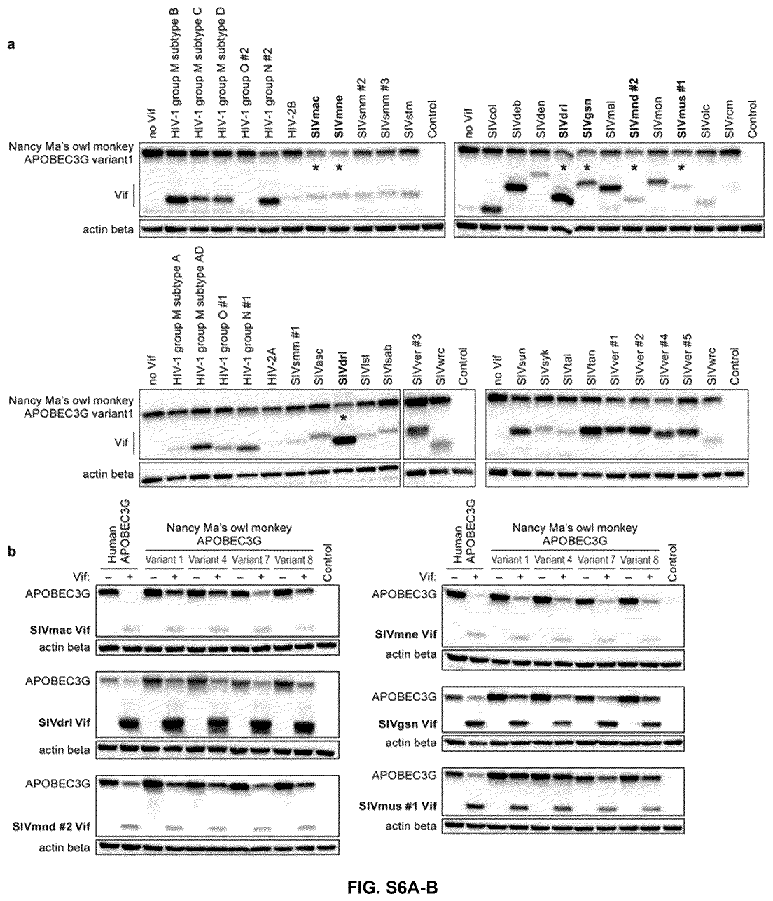

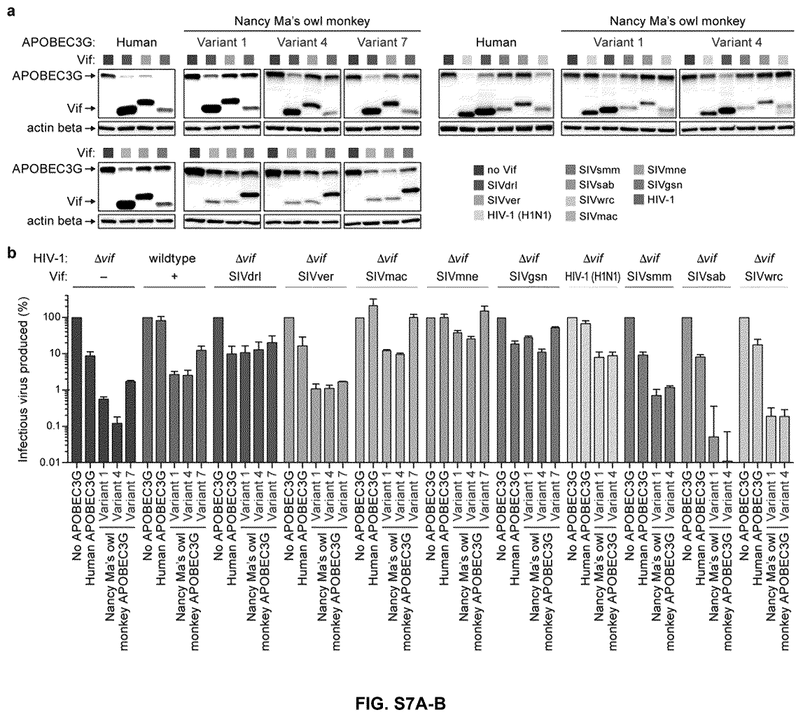

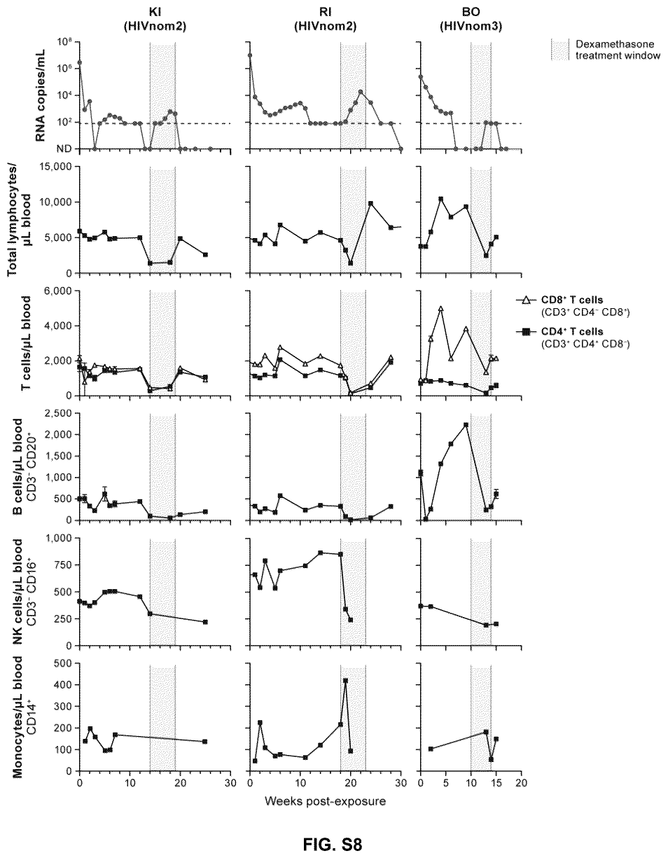

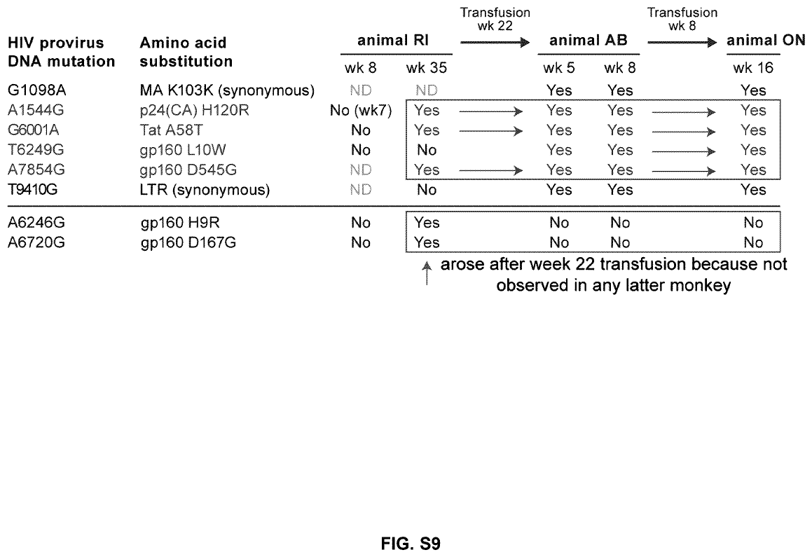

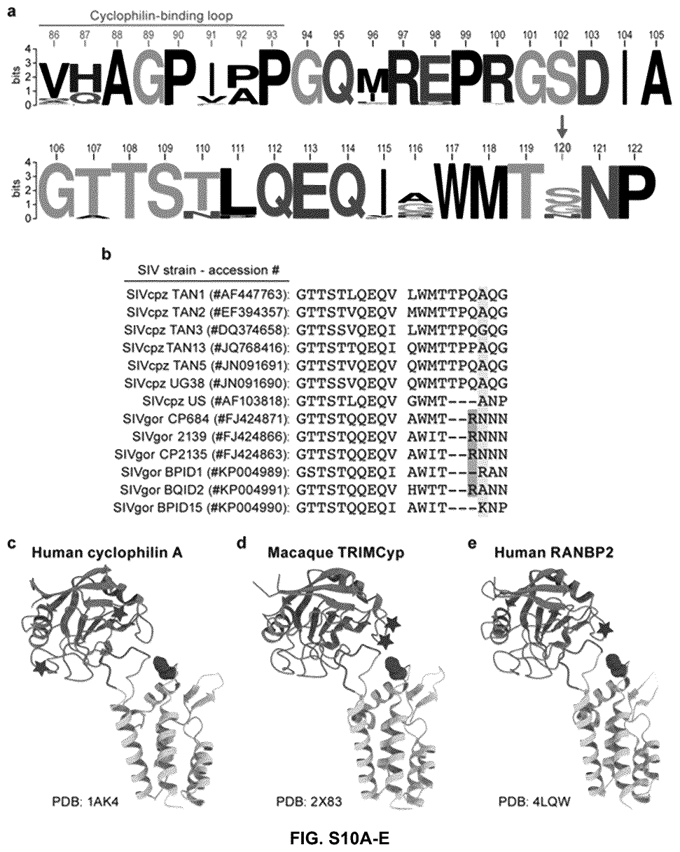

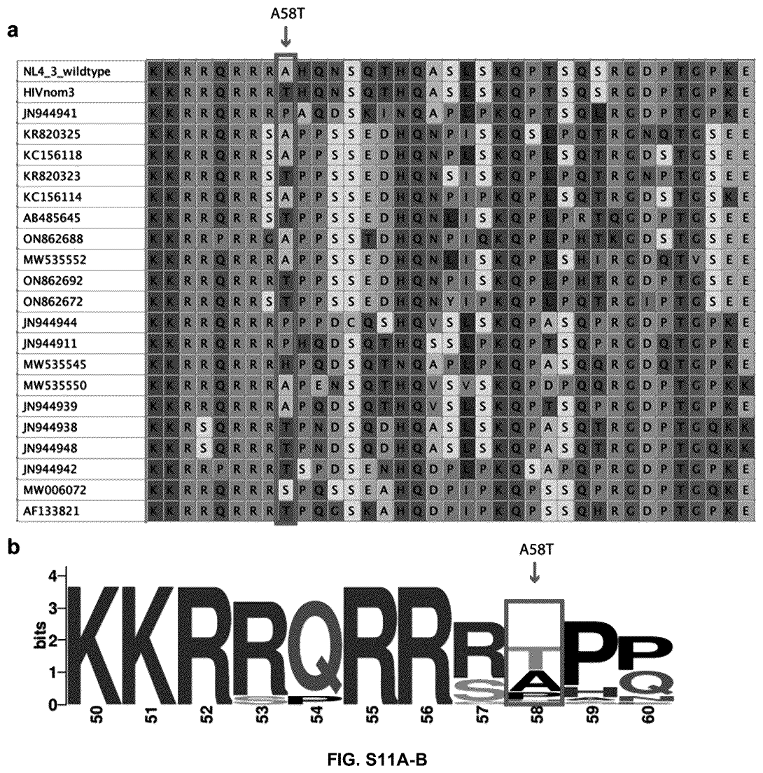

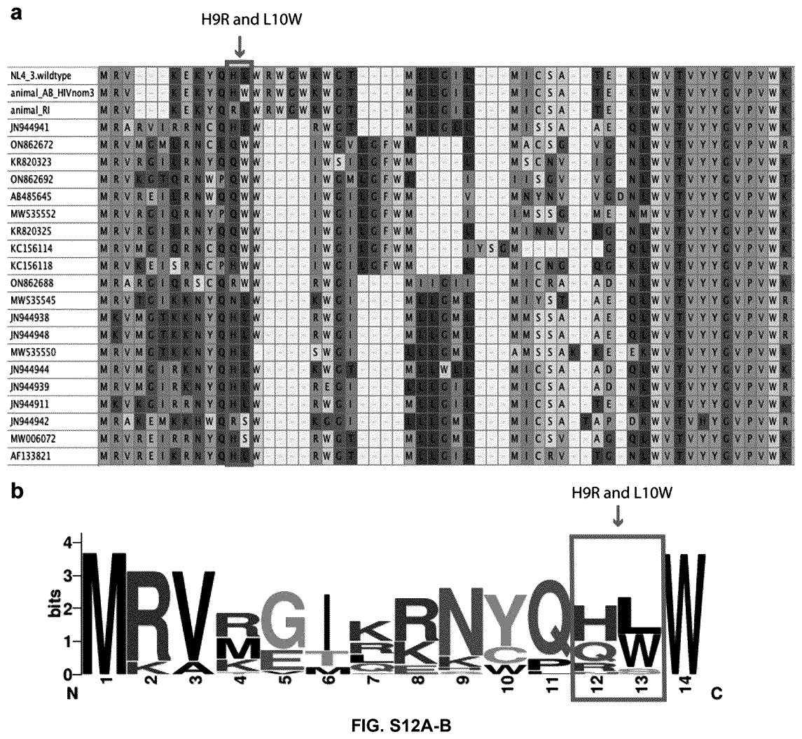

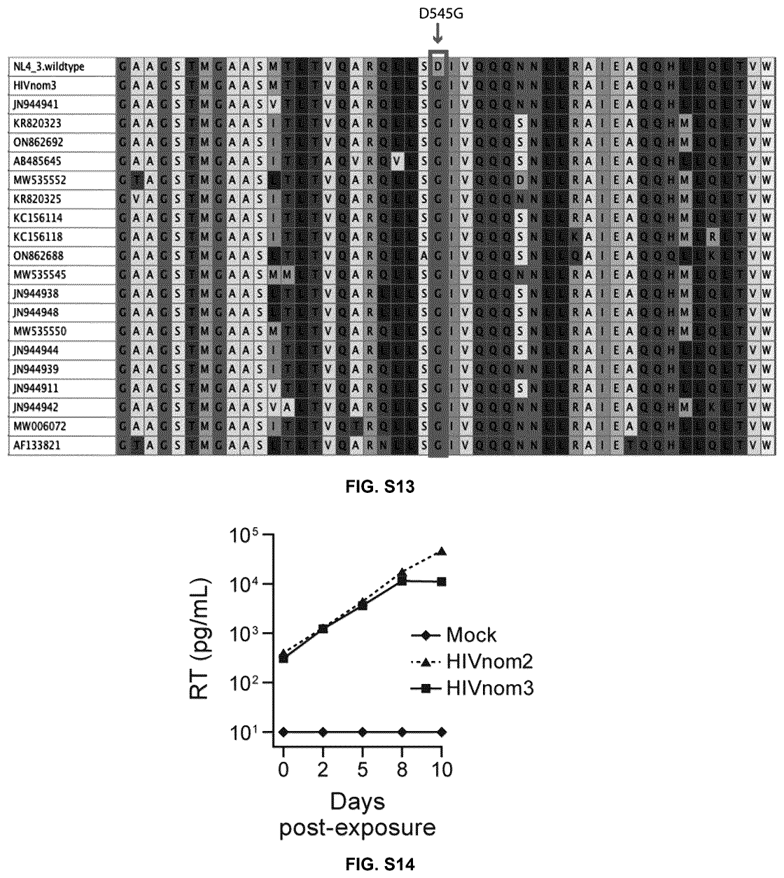

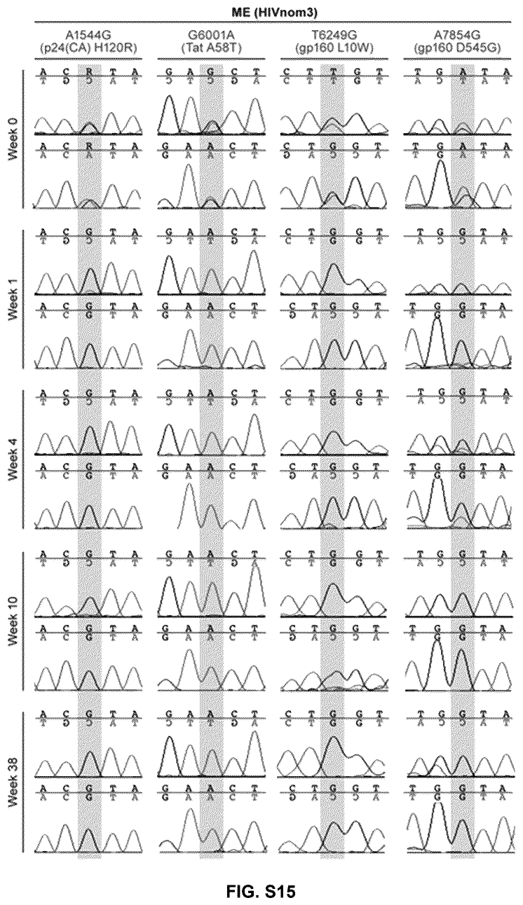

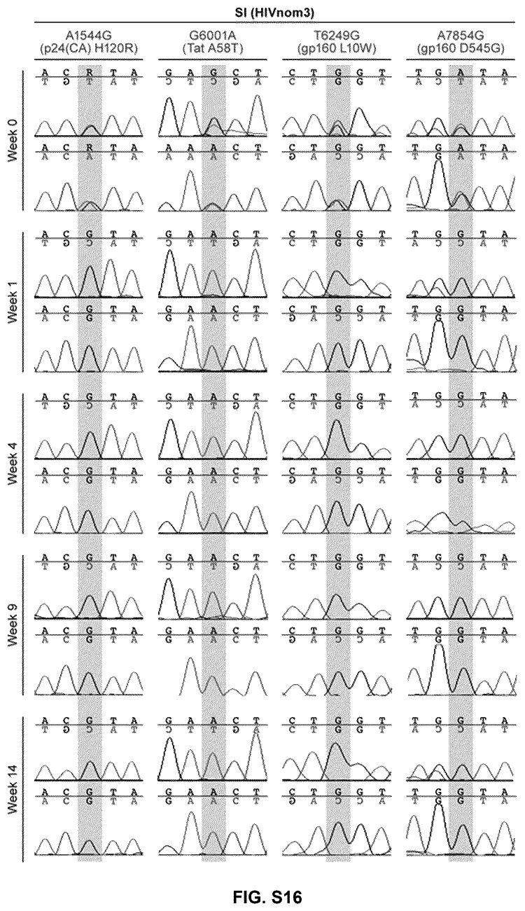

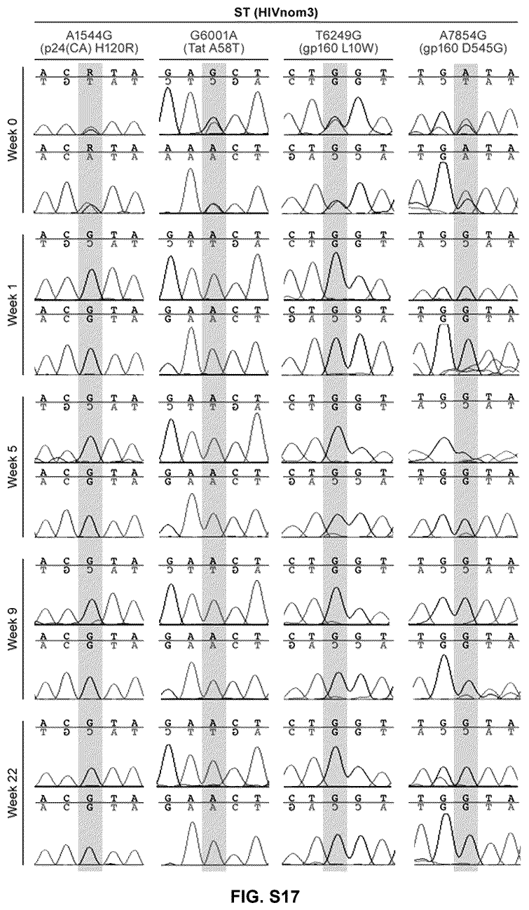

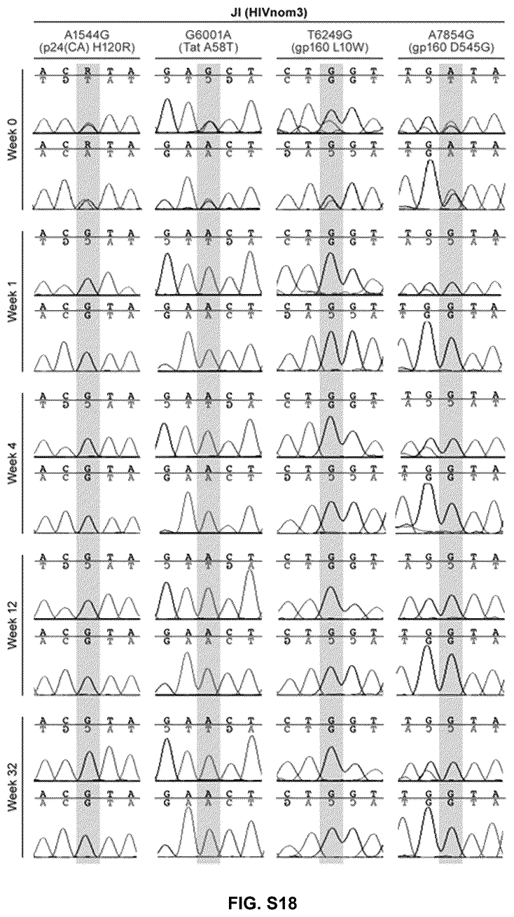

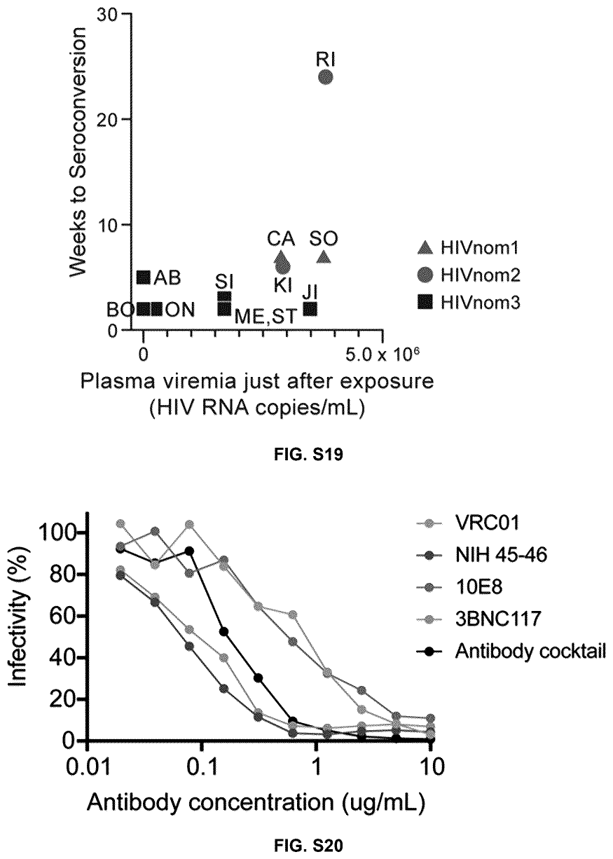

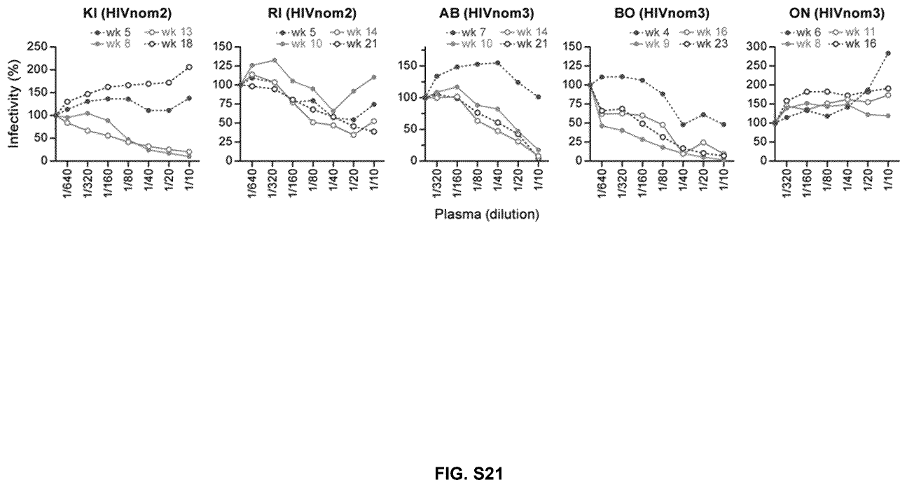

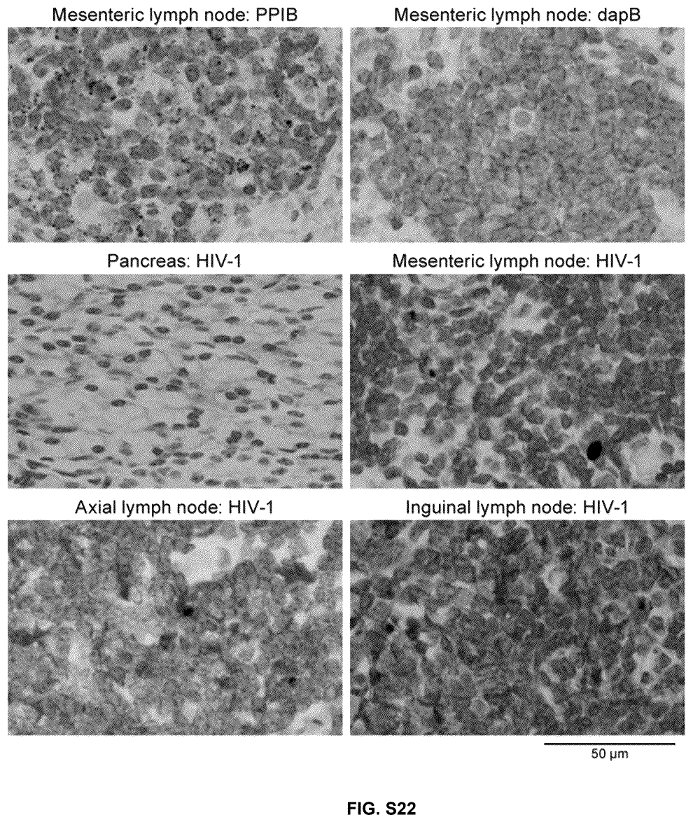

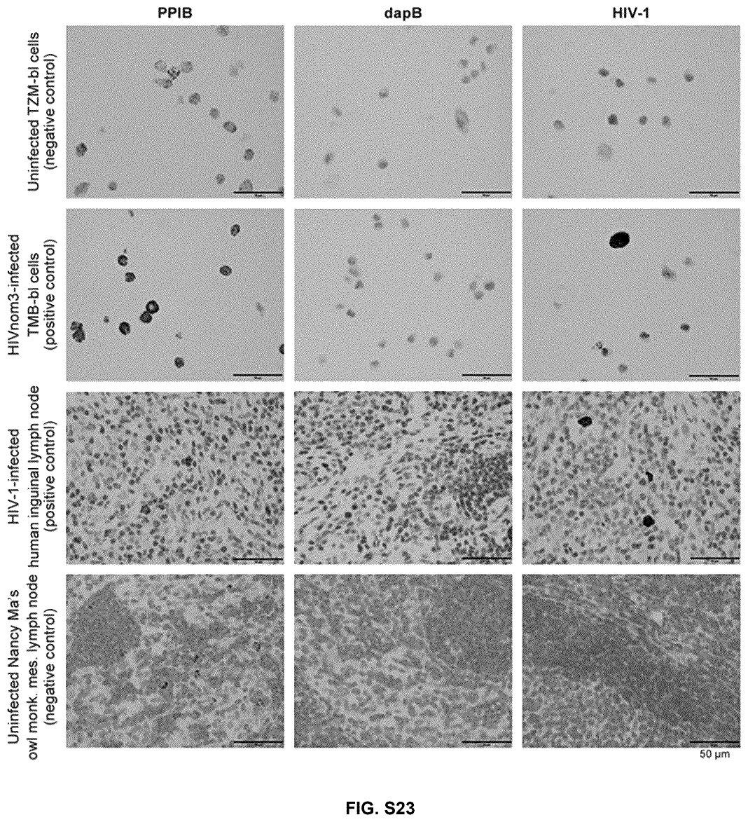

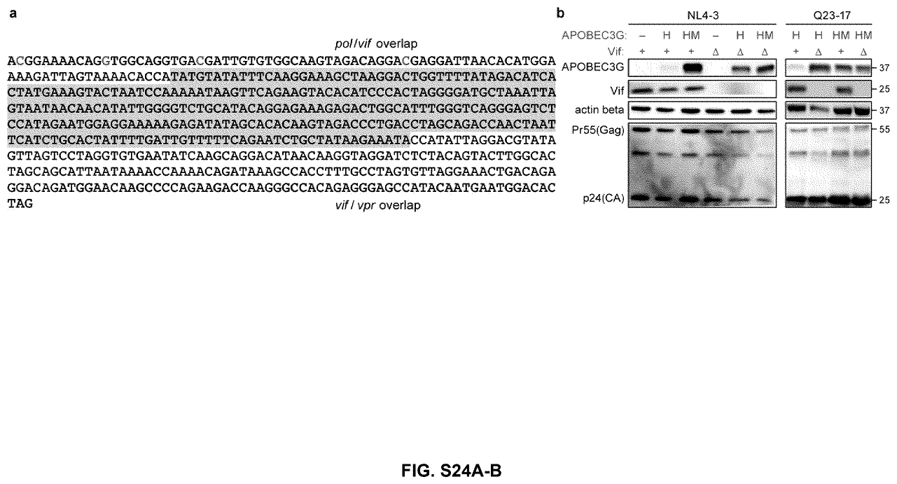

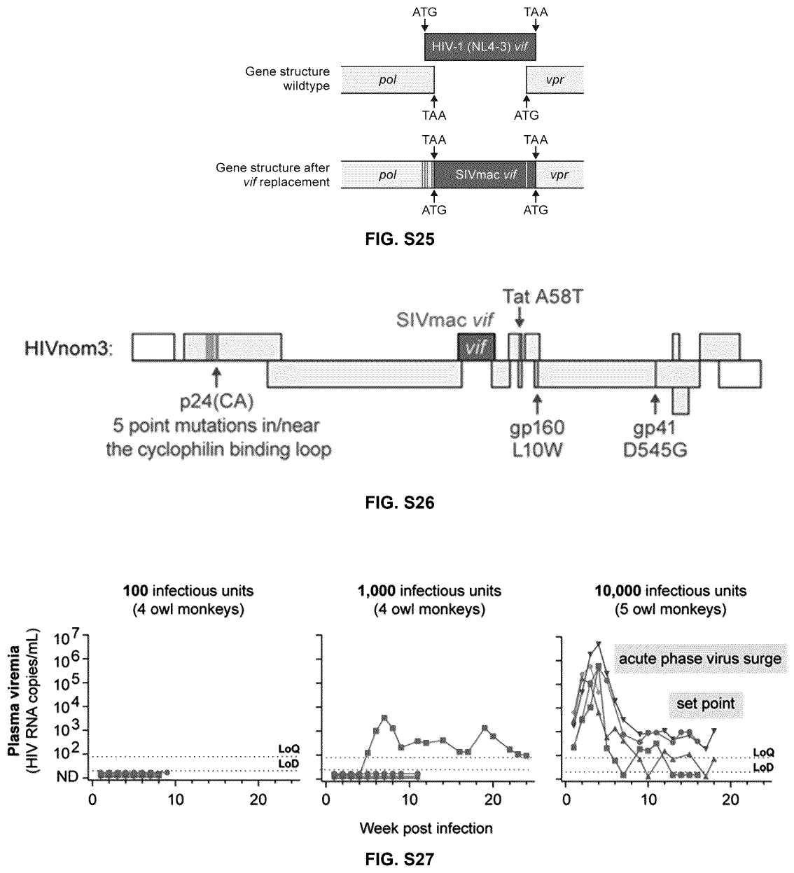

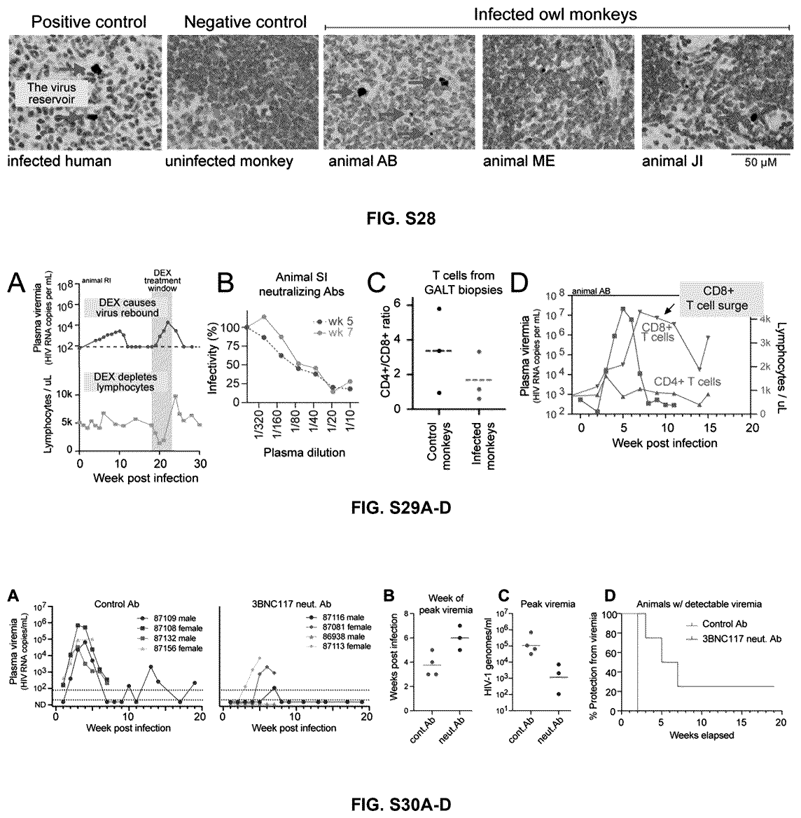

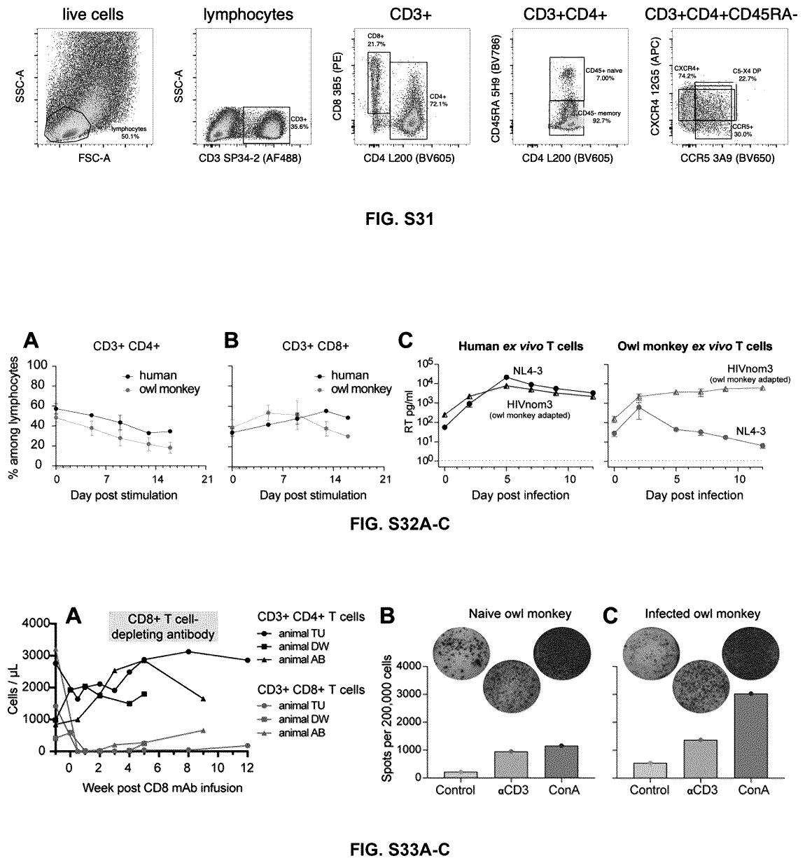

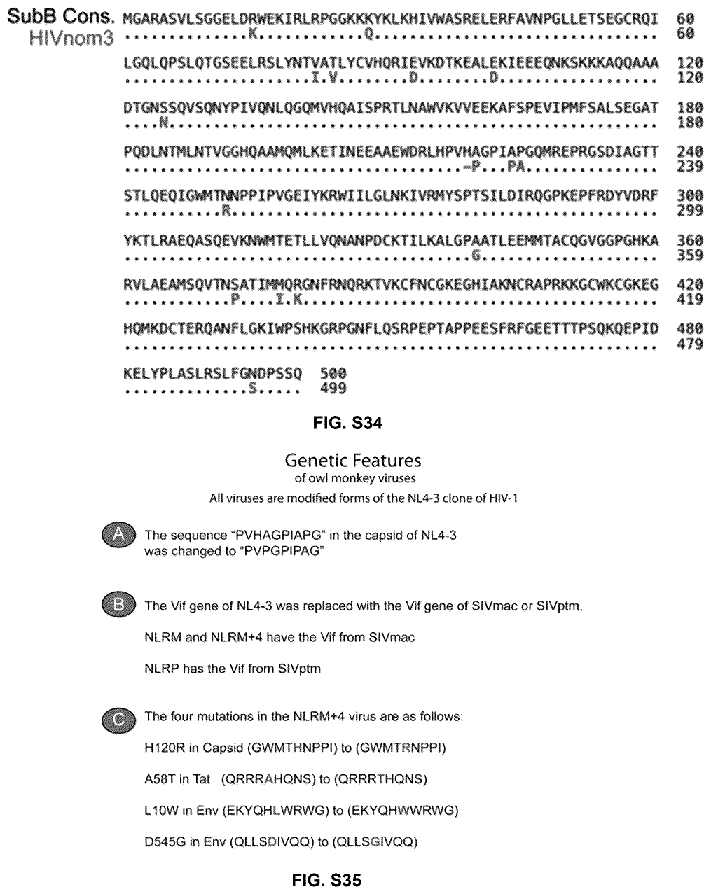

FIG. S21. Immunological and histological responses in HIVnom-infected Nancy Ma's owl monkeys. Neutralizing antibodies in plasma of the indicated animals determined as in FIG. 4b. Neutralization against autologous virus was assayed, and breadth of neutralization against other viruses was not tested.

FIG. S22. Animal JI. RNA in situ hybridization findings. Collective RNAscope in situ hybridization (ISH) photomicrographs from HIVnom3-infected animal JI. Positive results are indicated by brown staining. PPIB, positive control probe set recognizing the mRNA transcribed from the human peptidylprolyl isomerase B (PPIB) gene to assess RNA viability of the samples; dapB, negative control probe set targeting a bacterial gene transcript to assess background/nonspecific staining; HIV-1, a probe set tiled along the length of HIV-1 (NL4-3) RNA. The mesenteric lymph node section probed with dapB and the pancreas section incubated with the HIV-1 probe served as negative controls. Magnification 60×.

FIG. S23. Positive and negative control tissues utilized for RNA in situ hybridization optimization. Representative photomicrographs of various control tissues. In the top two rows, TZM-bl cells grown in culture were or were not exposed to HIVnom3. These cells serve as controls. In the bottom two rows, a lymph node from an HIV-1-infected human (positive control), or from an unexposed owl monkey (negative control) are also shown. PPIB, positive control probe set recognizing the mRNA transcribed from the human peptidylprolyl isomerase B (PPIB) gene to assess RNA viability of the samples; dapB, negative control probe set targeting a bacterial gene transcript to assess background/nonspecific staining; HIV-1, a probe set tiled along the length of HIV-1 (NL4-3) RNA. Magnification 60×. Positive results are indicated by brown staining.

FIG. S24A-B. Construction and testing of HIV-1 NL4-3Δvif and HIV-1 Q23-17Δvif proviral molecular clones. a, Full-length molecular clones for HIV-1 isolates NL4-3²⁵ and Q23-17⁶² were obtained from HIV Reagent Program. Gibson cloning was performed with three fragments to construct vif-deleted versions of these clones. Sequences highlighted in yellow represent the pol/vif overlap and vif/vpr overlap. Two of the Gibson fragments were PCR products amplified from the molecular clone to obtain products spanning from the ampicillin resistance gene in the plasmid to the pol/vif overlap (5′ fragment) and from the ampicillin resistance gene to the vif/vpr overlap (3′ fragment). The 5′ and 3′ fragments contain overlapping sequence in the ampicillin resistance gene. These PCR fragments were ligated to each other, and to a synthesized vif (IDT gBlock) spanning from the pol/vif overlap region to the vif/vpr overlap region (with complimentary sequence to the 5′ and 3′ PCR fragments described above on either end). The synthesized vif mutated all ATG sequences (letters in red), including the original vif start codon in the pol/vif overlap region. These changes introduced three synonymous and one nonsynonymous substitution at the 3′ end of pol. Sequence highlighted in green is the 284 internal bases of vif that were deleted in the synthesized version. b, HIV-1 NL4-3Δvif and Q23-17Δvif are functional and do not degrade human APOBEC3G. 293T cells were co-transfected with two plasmids: one expressing APOBEC3G (25 ng of plasmid encoding human “H” APOBEC3G or APOBEC3G D128K-denoted “HM” for “human mutant”) and one expressing an HIV-1 proviral clone (300 ng of plasmid expressing HIV-1 NL4-3, Q23-17, NL4-3Δvif or Q23-17Δvif). The mutant form of human APOBEC3G (D128K) is known to be resistant to Vif-mediated degradation. It was designed and made using NEBaseChanger and the standard protocol of the Q5 Site-Directed Mutagenesis Kit (New England Biolabs #E0554S). Forty-eight hours post transfection cells were collected, lysed, and subject to western blotting with antibodies to APOBEC3G (HA; Thermo Fisher Scientific #MA1-91878-HRP), Vif (BEI Resources #ARP-6459), and actin beta (Cell Signaling Technology #3700S). A goat anti-mouse horseradish peroxides-conjugated antibody (Promega #W4021) was used as a secondary probe for the Vif and actin beta blots. Applicants also probed with an anti-p24 (CA) antibody (BEI Resources #ARP-3537) as a proxy for viable viral production. These data are representative of two independent experiments. Note: The anti-p24 (CA) blot for HIV-1 NL4-3 was performed on whole cell extract of the 293T producer cells, whereas the anti-p24 (CA) blot for Q23-17 was performed on viruses isolated from the supernatants of the 293T producer cells.

FIG. S25. Replacement of HIV-1 (NL4-3) vif with that of SIVmne or SIVmac. Applicants used three-fragment Gibson cloning to replace the vif open reading frame of pNL4-3 with the vif from SIVmne (GenBank #U79412) or SIVmac (GenBank #M19499). The 5′ and 3′ proviral Gibson cloning fragments were produced as in FIG. S24. The synthesized DNA (gBlock) in this case, however, includes the full vif open reading from the SIVmne or SIVmac genomes. After assembly, the resulting provirus had the start codon of the macaque vifs immediately downstream of the HIV-1 pol stop codon, and the stop codon of the macaque vifs immediately upstream of the start codon of HIV-1 vpr (thus eliminating the pol/vif and vif/vpr open reading frame overlaps). The synthesized DNA also contained nucleotide substitutions to destroy all ATG sequences from the pol/vif overlap region (green lines), thus ensuring that vif is transcribed only from the macaque vif start codon. These changes resulted in three synonymous and one nonsynonymous change at the 3′ of pol. The start codon of SIVmac vpx (in what would be the vif/vpx genomic overlap in SIVmac) was similarly eliminated (white line).

FIG. S26. Genome schematic of HIVnom3, an owl-monkey adapted strain of HIV-1. The virus Applicants are using is based on HIV-1 isolate NL4-3. It contains 8 single point mutations (green) and the vif gene of SIVmac. This leaves 93% of the genome still identical to NL4-3. Green arrows point to single amino acid substitutions. The gp160 L10W mutation is in the signal peptide of Env.

FIG. S27. HIV-1 infection of owl monkeys. Owl monkeys were inoculated with HIVnom3, at three different doses shown at top, and with each line on each graph representing a different animal. Blood was drawn weekly for up to 24 weeks (˜6 months). Plasma viremia was determined by RT-qPCR (Y axis). Dotted horizontal lines depict the limit of quantification (LoQ) of 80 HIV-1 genomes per milliliter and the limit of detection (LoD). ND=not detected. At the highest dose, all monkeys become infected.

FIG. S28. The viral reservoir in owl monkey lymph nodes. RNAscope RNA in situ hybridization performed on lymph nodes from an HIV-1 infected human (left, positive control) or HIVnom3 infected owl monkeys. Representative micrographs are shown of formalin-fixed tissues stained with RNAscope probes that anneal to transcribed HIV-1 RNA. Positive results are represented by brown staining and emphasized with red arrows. Animal AB is particularly noteworthy as this animal had been non-viremic for 37 weeks (˜9 months) at necropsy, yet HIVnom3 RNA was clearly being transcribed within lymph nodes. Magnification 60×.

FIG. S29A-D. Evidence for T cell responses in infected owl monkeys. A. Plasma viremia in an infected monkey (top; Y axis). In this monkey, after 10 weeks the set point viremia falls to the limit of quantification (dotted line; 80 HIV-1 genomes per mL). At week 18, the monkey was administered dexamethasone (DEX; pink box), and viremia then rebounded. Lymphocyte levels drop during DEX treatment. B. Neutralization assay. Owl monkey plasma from 5 and 7 weeks after infection was serially diluted (X-axis) and pre-mixed with autologous HIVnom3. Virus entry into permissive cells (Y-axis), normalized to cells infected with just virus. C. GALT surgeries were performed on 3 uninfected and 3 infected monkeys, near the time of the acute phase virus surge. A relative reduction of CD4+ T cells in the GALT can be seen in infected monkeys, although more datapoints are needed (and being collected currently). D. Viral load (pink) and CD4+ and CD8+ T cell count (brown) across 15 weeks of infection.

FIG. S30A-D. A passive protection study in owl monkeys. In a study that is currently ongoing, owl monkeys were administered a single intravenous 40 mg/kg dose of an isotype control Ab or an HIV-1 neutralizing Ab (3BNC117). 24 hours later, all animals were infected with 10,000 infectious units of HIVnom3. A) Plasma viremia of the infected animals over time, with later datapoints being collected in ongoing experiments. B) The single dose of nAb elongated the time to peak viremia, and C) reduced the peak viremia when it did occur. D) Survival plot representing the animals from each arm.

FIG. S31. Gating strategy for flow cytometry. Gating strategies for the identification of HIVnom3 target cells in mononuclear cells isolated from owl monkey GALT (gut-associated lymphoid tissue) biopsies.

FIG. S32A-C. Growth and infection of T cells from owl monkeys. A, B) PMBCs from humans or owl monkeys were stimulated with IL-2 and PHA, and the percentage of lymphocytes that were CD3+CD4+ (A) or CD3+CD8+ (B) was enumerated over time using flow cytometry. C) HIV-1 and HIVnom3 replication over 12 days on PBMCs stimulated as in A, B. The owl monkey-adapted virus HIVnom3 grows on owl monkey and human T cells, while the parent NL4-3 virus grows only on human T cells, as expected.

FIG. S33A-C. CD8+ T cell depleting antibody and ELISPOT assay for owl monkeys. A) In three animals treated with a single dose of a monoclonal CD8 antibody (administered at week 0) Applicants observe sustained depletion of CD8+ T cells, but not CD4+ T cells, as determined by flow cytometry. B, C) Here Applicants show that a commercially available IFNγ ELISPOT assay by Mabtech can be used to measure owl monkey PMBC stimulation. PBMCs from B) naive or C) HIVnom3 infected animals were plated, stimulated for 48-hours (X axis), and assayed via the kit. Spots (Y axis and shown in images) indicate cells producing and secreting IFNγ.

FIG. S34. Alignment of Gag from the HIV-1 subtype B consensus and HIVnom3. BEI has a large collection of peptides available for the subtype B consensus sequence, spanning the entire length of HIV-1. Here, Applicants show an alignment of Gag from this consensus (SubB Cons) and from our virus (HIVnom3).

FIG. S35. Virus mutations identified in the present disclosure.

### DETAILED DESCRIPTION OF THE INVENTION

The present inventors describe herein a monkey species Aotus nancymaae (owl monkeys) infected with HIV-1. Owl monkeys are small docile animals that tolerate blood draws while awake, have no zoonotic pathogens, and that breed much faster than macaques. Owl monkeys become infected with HIV-1 modified variant that includes a heterologous Viral infectivity factor (Vif) and further includes a combination of point mutations in or near the cyclophilin binding loop of the capsid peptide (CA), but otherwise they are 100% unaltered HIV-1. Specifically, to infect owl monkeys, the present inventors modified HIV-1 with eight to nine non-synonymous point mutations and replacement of the Vif accessory gene. Notably, the virus is still 93% wildtype (compare with GenBank Accession No for NL4-3: AF324493, SEQ ID NO. 1, which include the vector backbone).

These relatively minor modifications allow the virus to bypass the owl monkey APOBEC3G and TRIM-Cyp restriction factors. Since SHIVs only include ˜30% of the HIV-1 genome, the owl monkey model is an enormous step forward in that it models HIV-1 itself, with all of the epitopes relevant to humoral and cell-mediated immunity, including, importantly, CD8+ T cells. Importantly, owl monkeys infected with HIV-1 recapitulate infection as is observed in humans: an acute phase of infection with plasma viremia up to 10⁷ copies/mL, subsequent control of the virus, and seroconversion. This model will enable the study of HIV-1 for the first time in a primate model and represents an exciting new platform for vaccine and cure development.

For studies of immunity and for vaccine development, the present model represents an advancement over prior SHIV models. It is now clear that both antibody- and CD8+ T cell-based immunity are critical to HIV-1 protection and control. Antibodies can counteract viruses in numerous ways, and CD8+ T cells are important for killing HIV-infected cells. However, in SHIV's, only ˜30% of the HIV-1 genome is present (essentially just Env). Thus, SHIVs do not contain all of the epitopes relevant to humoral and cell-mediated immunity. An authentic HIV-1 challenge model like the owl monkey can enable testing of more diverse vaccine approaches beyond Env immunogens. Moreover, the owl monkey model correctly captures all key events in HIV-1 infection: transmission, acute infection, seroconversion, and establishment of the latent reservoir. Furthermore, the present inventors have established a high-quality owl monkey genome project and have identified and optimized necessary reagents, antibodies, and diagnostics required for HIV-1 research in this species. In one preferred embodiment, the invention can include generation of a transmitted-founder (T/F) viruses (the viruses that start new infections) representing major global subtypes. The viruses can be important for developing vaccines and cures in this new model, and will also allow for the study of additional aspects of HIV-1 transmission, protective immunity, and the latent reservoir.

Another embodiment of the invention includes the creation of a novel HIV-1 model animal, and preferably an owl monkey, infected with one or more novel HIV-1 variants of the invention. The invention may further include methods of screening or testing the efficacy of one or more potential therapeutic compounds, or other therapies directed to HIV-1. In one preferred embodiment, a transgenic, non-human animal, and preferably an owl monkey can be infected by one or more novel HIV-1 variants of the invention, namely HIVom1, HIVom1*, HIVom2, or HIVon2* that exhibits at least one phenotype associated with HIV-1 infection may be established. (See also Naming Convention Section for alternative name of HIV-1 strains).

Next, and preferably a therapeutically effective amount of a therapeutic compound directed to the treatment of one or more pathological phenotypes associated HIV-1 may be administered to the animal to determine if the therapeutic compound decreases one or more phenotypes associated with HIV-1, and comparing any phenotype changes with an animal that did not receive the therapeutic compound. Administration may be accomplished through a variety of routes, including vaginal, rectal, oral, nasal, injection, and the like. Moreover, a therapeutic compound may include one or more small molecules, such as inhibitors of protein function or gene expression, or may include one or more biologic therapeutics, such as monoclonal or other antibody based treatments. In a preferred embodiment, the therapeutic compound may include a vaccine against HIV-1. Notably, a therapeutic compound may be part of a pharmaceutical composition, having a pharmaceutical carrier, which would be known by one of ordinary skill in the art.

The present invention includes a plurality of HIV-1 variants configured to infect owl monkeys, which may be selected from the strains: HIVom1 (ATCC Patent Deposit No. PTA-127572), HIVom1* (ATCC Patent Deposit No. PTA-127573), HIVom2 (ATCC Patent Deposit No. PTA-127574), and HIVom2* (ATCC Patent Deposit No. PTA-127575) as described herein. (See also Naming Convention Section for alternative name of HIV-1 strains).

In one embodiment, the novel HIV-1 variant of the invention includes a genetically modified capsid region having one or more mutations at or near the cyclophilin-binding loop. In this preferred embodiment, one or more mutations to the cyclophilin-binding loop can be positioned between positions 87 and 93 according to SEQ ID NO. 3 of the cyclophilin-binding loop, and preferably positions: 87, 88, 92, and/or 93 of the cyclophilin-binding loop, according to SEQ ID NO. 3, and even more preferably:
deletion of a histidine residue at position 87 of the cyclophilin-binding loop;
a proline substituted at position 88 of the cyclophilin-binding loop;
a proline substituted at position 92 of the cyclophilin-binding loop;
an alanine substituted at position 93 of the cyclophilin-binding loop; or
or any conserved amino acid substitution between positions 87 and 93 according to SEQ ID NO. 3.

In this preferred embodiment, the mutations of the cyclophilin-binding loop position comprises amino acids deletions or substitutions embodied in the amino acid sequence SEQ ID NO. 9, wherein the mutations include:
a ΔH87 mutation, wherein a histidine residue at position 87 of the cyclophilin-binding loop is deleted;
an A88P mutation, wherein an alanine residue of the cyclophilin-binding loop is replaced with a proline;
an A92P mutation, wherein an alanine residue of the cyclophilin-binding loop is replaced with a proline; and
a P93A mutation, wherein a proline is of the cyclophilin-binding loop replaced with an alanine residue or
or any conserved amino acid substitution at positions 87-88 and 92-93 according to SEQ ID NO. 3.

In certain alternative embodiments, the invention may include a pharmaceutical composition comprising one or more HIV-1 variants having a capsid protein according to the amino acid sequence SEQ ID NO. 9, or 11, and a pharmaceutically acceptable carrier. In further embodiments, the invention may include an isolated nucleotide sequence, operably linked to a promoter, encoding the genetically modified capsid peptide according to the amino acid sequence SEQ ID NO. 9, or 11, which may further be incorporated into an expression vector. In still further embodiments, the invention may include an isolated nucleotide sequence, operably linked to a promoter, encoding an HIV-variant including a genetically modified capsid peptide according to the amino acid sequence SEQ ID NO. 9, or 11, which may further be incorporated into an expression vector.

The present invention includes a novel HIV-1 variant having a heterologous viral infectivity factor (Vif). In a preferred embodiment, the HIV-1 variant of the invention is genetically modified to replace the wild-type Vif (SEQ ID NO. 2), with a heterologous Vif, preferably selected from a simian immunodeficiency virus (SIVVif). In one embodiment, the SIVVif of the invention is selected from: SEQ ID NO. 6, or SEQ ID NO. 7, or a sequence having at least 85% sequence homology, or a fragment or variant thereof. In this embodiment, the SIVVif is inserted upstream of a Pol region and downstream of a Vpr region, such that SIVVif includes a start codon inserted downstream of a stop codon of the Pol region, and a stop codon inserted upstream of a start codon of the Vpr region, and wherein an internal start codon positioned within said SIVV if is disrupted, for example through site-directed mutagenesis. In this configuration, the HIV-1 variant may further be modified such that one or more start codons positioned within the Vif/Vpr overlap region are disrupted, and further one or more start codons positioned within the Pol/Vif overlap region are disrupted.

In certain alternative embodiments, the invention may include a pharmaceutical composition comprising one or more HIV-1 variants having a heterologous SIVVif protein according to the amino acid sequence SEQ ID NO. 6, or SEQ ID NO. 7, or a sequence having at least 85% sequence homology, or a fragment or variant thereof, and a pharmaceutically acceptable carrier. In further embodiments, the invention may include an isolated nucleotide sequence, operably linked to a promoter, encoding the SIVVif protein according to the amino acid sequence SEQ ID NO. 6, or SEQ ID NO. 7, or a sequence having at least 85% sequence homology, which may further be incorporated into an expression vector. In still further embodiments, the invention may include an isolated nucleotide sequence, operably linked to a promoter, encoding an HIV-variant including SIVVif protein according to the amino acid sequence SEQ ID NO. 6, or SEQ ID NO. 7, or a sequence having at least 85% sequence homology, which may further be incorporated into an expression vector.

The present invention includes a novel HIV-1 variant having one or more mutations that modulate infectivity of the variant. In a preferred embodiment, the one or more mutations of the invention are present in the Capsid, Tat, and Envelope (Env) proteins. In this embodiment, the novel HIV-1 variant of the invention includes one or more mutations selected from:
a substitution mutation at position 120 of the HIV-1 Capsid protein according to SEQ ID NO. 3;
a substitution mutation at position 58 of the HIV-1 Tat protein according to SEQ ID NO. 4;
a substitution mutation at position 9 or 10 of the HIV-1 Env protein according to SEQ ID NO. 5;
a substitution mutation at position 545 of the HIV-1 Env protein according to SEQ ID NO. 5,
a substitution mutation at position 167 of the HIV-1 Env protein according to SEQ ID NO. 5; or
a combination of the same.

In still further embodiments, the novel HIV-1 variant of the invention includes one or more mutations selected from:
an arginine substituted at position 120 of the HIV-1 Capsid protein according to SEQ ID NO. 3;
a threonine substituted at position 58 of the HIV-1 Tat protein according to SEQ ID NO. 4;
an arginine substituted at position 9 of the HIV-1 Env protein according to SEQ ID NO. 5,
a tryptophan at position 10 of the HIV-1 Env protein according to SEQ ID NO. 5; and
a glycine substituted at position 545 of the HIV-1 Env protein according to SEQ ID NO. 5, or a combination of the same.
a glycine substituted at position 167 of the HIV-1 Env protein according to SEQ ID NO. 5, or a combination of the same.

In still further embodiments, the novel HIV-1 variant of the invention includes one or more of mutation is selected from:
a H120R substitution of the HIV-1 Capsid protein according to SEQ ID NO. 10;
a A58T substitution of the HIV-1 Tat protein according to SEQ ID NO. 12;
a H9R substitution of the HIV-1 Env protein according to SEQ ID NO. 13;
L10W substitution of the HIV-1 Env protein according to SEQ ID NO. 14;
D545G substitution of the HIV-1 Env protein according to SEQ ID NO. 15,
a D167G of the HIV-1 Env protein according to SEQ ID NO. 20; or
a combination of the same.

Notably, in some embodiments, the non-modified bases can be variable and include one or more conservative substitution such that in some embodiments, the invention can include a capsid, or other modified peptide described herein, wherein the point mutation or deletion is conserved, but the intervening sequence can include a sequence having between at least 85%-99% sequence homology of the same.

In certain alternative embodiments, the invention may include a pharmaceutical composition comprising one or more HIV-1 variants having a one more proteins is selected from:
a H120R substitution of the HIV-1 Capsid protein according to SEQ ID NO. 10;
a A58T substitution of the HIV-1 Tat protein according to SEQ ID NO. 12;
a H9R substitution of the HIV-1 Env protein according to SEQ ID NO. 13;
L10W substitution of the HIV-1 Env protein according to SEQ ID NO. 14;
D545G substitution of the HIV-1 Env protein according to SEQ ID NO. 15,
a glycine substituted at position 167 of the HIV-1 Env protein according to SEQ ID NO. 20;
or combination of the same; and
a pharmaceutically acceptable carrier.

In further embodiments, the invention may include an isolated nucleotide sequence, operably linked to a promoter, encoding the one or more proteins having: an arginine substituted at position 120 of the HIV-1 Capsid protein; a threonine substituted at position 58 of the HIV-1 Tat protein; an arginine substituted at position 9 of the HIV-1 Env protein a tryptophan at position 10 of the HIV-1 Env protein; and a glycine substituted at position 545 of the HIV-1 Env protein, a glycine substituted at position 167 of the HIV-1 Env protein, or a combination of the same, which may further be incorporated into an expression vector. In still further embodiments, the invention may include an isolated nucleotide sequence, operably linked to a promoter, encoding an HIV-variant including one or more proteins having: an arginine substituted at position 120 of the HIV-1 Capsid protein; a threonine substituted at position 58 of the HIV-1 Tat protein; an arginine substituted at position 9 of the HIV-1 Env protein a tryptophan at position 10 of the HIV-1 Env protein; and a glycine substituted at position 545 of the HIV-1 Env protein, a glycine substituted at position 167 of the HIV-1 Env protein, or a combination of the same, which may further be incorporated into an expression vector.

In certain alternative embodiments, the invention may include a HIV-1 variant having one more proteins according to the amino acid sequence SEQ ID NO.'s 6-20, and a pharmaceutically acceptable carrier. In further embodiments, the invention may include an isolated nucleotide sequence, operably linked to a promoter, encoding the one or more proteins according to the amino acid sequence SEQ ID NO.'s 6-20, which may further be incorporated into an expression vector. In still further embodiments, the invention may include an isolated nucleotide sequence, operably linked to a promoter, encoding an HIV-variant including one or more proteins according to the amino acid sequence SEQ ID NO.'s 10-16, which may further be incorporated into an expression vector.

The present invention includes one or more genetically modified HIV-1 variants adapted to infect Aotus nancymaae (owl monkey). The HIV-1 variant of the invention includes a modified capsid peptide according to SEQ ID NO. 3, wherein the cyclophilin-binding loop is substituted with the binding cyclophilin binding loops simian immunodeficiency viruses (SIVs), and in a preferred embodiment simian immunodeficiency viruses (SIVs). In a preferred embodiment, the HIV-1 variant of the invention is adapted to infect an owl monkey includes a modified capsid peptide according to SEQ ID NO. 9, which may encode a capsid peptide having a cyclophilin-binding loop with the following mutations:
a ΔH87 mutation, wherein a histidine residue at position 87 of the cyclophilin-binding loop is deleted;
an A88P mutation, wherein an alanine residue of the cyclophilin-binding loop is replaced with a proline;
an A92P mutation, wherein an alanine residue of the cyclophilin-binding loop is replaced with a proline;
a P93A mutation, wherein a proline is of the cyclophilin-binding loop replaced with an alanine residue; and
a heterologous simian immunodeficiency virus Viral infectivity factor (SIVVif) selected from: SEQ ID NO. 6, or SEQ ID NO. 7, or a sequence having between at least 85%-99% sequence homology, inserted upstream of a Pol region and downstream of a Vpr region.

In this embodiment, the SIVVif of the invention may include a start codon inserted downstream of a stop codon of the Pol region, and a stop codon inserted upstream of a start codon of the Vpr region, and wherein an internal start codon positioned within said SIVV if is disrupted. Additionally, one or more start codons positioned within the Vif/Vpr and Pol/Vif overlap regions can be disrupted.

In certain alternative embodiments, the invention may include a HIV-1 variant having a modified capsid peptide according to SEQ ID NO. 9, and a SIVVif protein according to the amino acid sequence SEQ ID NO. 6, or SEQ ID NO. 7, or a sequence having at least 85% sequence homology, and a pharmaceutically acceptable carrier. In further embodiments, the invention may include an isolated nucleotide sequence, operably linked to a promoter, encoding the modified capsid peptide according to SEQ ID NO. 9, and a SIVVif protein according to the amino acid sequence SEQ ID NO. 6, or SEQ ID NO. 7, or a sequence having at least 85% sequence homology, which may further be incorporated into an expression vector. In still further embodiments, the invention may include an isolated nucleotide sequence, operably linked to a promoter, encoding an HIV-variant including a modified capsid peptide according to SEQ ID NO. 9, and a SIVVif protein according to the amino acid sequence SEQ ID NO. 6, or SEQ ID NO. 7, or a sequence having at least 85% sequence homology, which may further be incorporated into an expression vector.

The present invention includes one or more genetically modified HIV-1 variants adapted to infect Aotus nancymaae (owl monkey). In a preferred embodiment, the HIV-1 variant of the invention includes a modified capsid peptide according to SEQ ID NO. 9, which encodes a cyclophilin-binding loop with the following mutations: a ΔH87 mutation, wherein a histidine residue at position 87 is deleted; an A88P mutation, wherein an alanine residue of the cyclophilin-binding loop is replaced with a proline; an A92P mutation, wherein an alanine residue of the cyclophilin-binding loop is replaced with a proline; a P93A mutation and a simian immunodeficiency virus Viral infectivity factor (SIVVif) selected from: SEQ ID NO. 6, or SEQ ID NO. 7, or a sequence having at least 85% sequence homology, preferably inserted upstream of a Pol region and downstream of a Vpr region, and one or more additional mutations selected from:
a H120R substitution of the HIV-1 Capsid protein according to SEQ ID NO. 10;
a A58T substitution of the HIV-1 Tat protein according to SEQ ID NO. 12;
a H9R substitution of the HIV-1 Env protein according to SEQ ID NO. 13;
a L10W substitution of the HIV-1 Env protein according to SEQ ID NO. 14;
a D545G substitution of the HIV-1 Env protein according to SEQ ID NO. 15;
a D167G substitution of the HIV-1 Env protein according to SEQ ID NO. 15; or
or a combination of the same.

In additional embodiments, the present invention includes one or more genetically modified HIV-1 variants adapted to infect Aotus nancymaae (owl monkey). In a preferred embodiment, the HIV-1 variant of the invention includes:
a modified capsid peptide according to SEQ ID NO.'s 9-11, or a sequence having between 85%-99% sequence homology with SEQ ID NO.'s 9-11;
a simian immunodeficiency virus Viral infectivity factor (SIVVif) selected from: SEQ ID NO.'s 6-7, or a sequence having between 85%-99% sequence homology with SEQ ID NO.'s 6-7;
and one or more additional mutations selected from:
   a H120R substitution of the HIV-1 Capsid protein according to SEQ ID NO. 10, or a sequence having between 85%-99% sequence homology with;
   a A58T substitution of the HIV-1 Tat protein according to SEQ ID NO. 12, or a sequence having between 85%-99% sequence homology with;
   a H9R substitution of the HIV-1 Env protein according to SEQ ID NO. 13, or a sequence having between 85%-99% sequence homology with SEQ ID NO. 13;
   a L10W substitution of the HIV-1 Env protein according to SEQ ID NO. 14, or a sequence having between 85%-99% sequence homology with SEQ ID NO. 14;
   a D545G substitution of the HIV-1 Env protein according to SEQ ID NO. 15, or a sequence having between 85%-99% sequence homology with SEQ ID NO. 15;
   a D545G, a H9R, and a L10W, substitution of the HIV-1 Env protein according to SEQ ID NO. 18, or a sequence having between 85%-99% sequence homology with SEQ ID NO. 18; or
   a D167G substitution of the HIV-1 Env protein according to SEQ ID NO. 19, or a sequence having between 85%-99% sequence homology with SEQ ID NO. 19;
   a D545G, a H9R,a L10W, and a D167G substitution of the HIV-1 Env protein according to SEQ ID NO. 20, or a sequence having between 85%-99% sequence homology with SEQ ID NO. 20 outside of the conserved mutations.

In this embodiment, the SIVVif of the invention may include a start codon inserted downstream of a stop codon of the Pol region, and a stop codon inserted upstream of a start codon of the Vpr region, and wherein an internal start codon positioned within said SIVV if is disrupted. Additionally, one or more start codons positioned within the Vif/Vpr and Pol/Vif overlap regions can be disrupted.

As noted above, in still further embodiments of the invention, one or more of the HIV variants of the invention can be administered to a mammal causing an infection, wherein the animal is preferably an Aotus nancymaae (owl monkey). Additional embodiments may include contacting a biological sample, such as a cell, tissue of bodily fluid sample from owl monkey with one or more of the HIV variants of the invention. Still further embodiments may include extracting a biological sample, such as a cell, tissue of bodily fluid sample from owl monkey that has been exposed to, or infected with one or more of the HIV variants of the invention.

In additional embodiments, one or more of the HIV variants of the invention can be administered to a mammal causing an infection, wherein the animal is preferably an Aotus nancymaae (owl monkey), and subsequently the resulting immunological and physiological responses measured. In additional embodiments, a therapeutic agent directed prevent HIV infection can be administered to a mammal, and preferably an Aotus nancymaae (owl monkey), and subsequently the owl money can be “challenged” with a HIV-1 variant of the invention and the resulting immunological response or protective effects measured. In a preferred embodiment, the therapeutic agent comprises a vaccine. If the vaccine is effective in generating a prophylactic immunological response that produces immunity in the subject against one or more of the “challenge” HIV-1 variants of the invention, that vaccine can be further pursued for use in a human subject. In alternative embodiments, one or more additional doses of a vaccine can be administered as a booster and the subsequent response evaluate in response to another challenge by one of the HIV-1 variants of the invention as described above. If, on the other hand the vaccine is ineffective in generating a prophylactic immunological response in the subject against one or more of the “challenge” HIV-1 variants of the invention, that vaccine can be reevaluated, modified or abandoned.

In additional embodiments, a therapeutic agent, such as a therapeutic small molecule or biologic directed to treat or prevent HIV infection is administered to a mammal and preferably an Aotus nancymaae (owl monkey), and either prior to administration of the therapeutic agent or subsequent to administration, the infected owl money can be “challenged” with a HIV-1 variant of the invention and the resulting physiological responses measured. In a preferred embodiment, the therapeutic agent comprises a therapeutic compound configured to treat or cure HIV infection, or a prophylactic compound configured to prevent HIV infection. Naturally, in certain embodiments, the therapeutic agent can be administered prior to administering the “challenge” HIV-1 variant, while in alternative embodiments, the therapeutic agent can be administered concurrent with, or even after administering the “challenge” HIV-1 variant. Moreover, the therapeutic agent can be administered as part of a dosing regimen based on the type of agent, predicted or observed response, and other variables that would be understood by one of ordinary skill. If the agent is effective in preventing infection, or treats one or more symptoms of infection by the one or more of the “challenge” HIV-1 variants of the invention, that agent can be further pursued for use in a human subject. If, on the other hand the agent is ineffective at preventing or treating infection by one or more of the “challenge” HIV-1 variants of the invention, that agent can be reevaluated, modified or abandoned.

The following definitions are provided to aid the reader in understanding the various aspects of the present disclosure. Unless defined otherwise, all technical and scientific terms used herein have the same meaning as commonly understood by those of ordinary skill in the art to which the disclosure pertains. Specifically, definitions of common terms in cell biology and molecular biology can be found in The Encyclopedia of Molecular Biology, published by Blackwell Science Ltd., 1994 (ISBN 0-632-02182-9; Benjamin Lewin, Genes X, published by Jones & Bartlett Publishing, 2009 (ISBN-10:0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds. Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

As used herein, “HIV-1” means the human immunodeficiency virus type-1. HIV-1 includes but is not limited to extracellular virus particles and the forms of HIV-1 associated with HIV-1 infected cells. As also used herein, “virus” and/or “virion” can mean either HIV-1 or HIV-1 viral particles, or viral peptide sub-units.

Mutations in the HIV-1 refer to any of point mutations, additions, deletions (though preferably not in the cleavage domain), and rearrangements. Mutations may be at a single site or at multiple sites in the HIV-1 genome. Mutations can be generated by standard techniques including random mutagenesis, targeted genetics and other methods know by those of ordinary skill in the art.

“Pharmaceutical compositions” are compositions that include an amount (for example, a unit dosage) of the disclosed compound(s) together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition). In one embodiment, a pharmaceutical compositions of the invention may include a quantity of HIV-1, and a pharmaceutically acceptable carrier, such as a pharmaceutically acceptable excipient or carriers

Such pharmaceutical compositions/formulations are useful for administration to a subject, in vivo or ex vivo. Pharmaceutical compositions and formulations include carriers or excipients for administration to a subject. As used herein the terms “pharmaceutically acceptable” and “physiologically acceptable” mean a biologically compatible formulation, gaseous, liquid, or solid, or mixture thereof, which is suitable for one or more routes of administration, in vivo delivery, or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules, and crystals. Supplementary active compounds (e.g., preservatives, antibacterial, antiviral, and antifungal agents) can also be incorporated into the compositions. The formulations may, for convenience, be prepared or provided as a unit dosage form. In general, formulations are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein.

Pharmaceutical formulations and delivery systems appropriate for the compositions and methods of the invention are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N.Y., pp. 253-315). For example, pharmaceutical compositions can optionally be formulated to be compatible with a particular route of administration. Exemplary routes of administration include administration to a biological fluid, an immune cell (e.g., T or B cell) or tissue, mucosal cell or tissue (e.g., mouth, buccal cavity, labia, nasopharynx, esophagus, trachea, lung, stomach, small intestine, vagina, rectum, or colon), neural cell or tissue (e.g., ganglia, motor or sensory neurons) or epithelial cell or tissue (e.g., nose, fingers, cars, cornea, conjunctiva, skin or dermis). Thus, pharmaceutical compositions include carriers (excipients, diluents, vehicles, or filling agents) suitable for administration to any cell, tissue, or organ, in vivo, ex vivo (e.g., tissue or organ transplant) or in vitro, by various routes and delivery, locally, regionally, or systemically.

Exemplary routes of administration for contact or in vivo delivery of a target inhibitor, is a dosage of the compound that is sufficient to achieve a desired therapeutic effect, such as can optionally be formulated include inhalation, respiration, intubation, intrapulmonary instillation, oral (buccal, sublingual, mucosal), intrapulmonary, rectal, vaginal, intrauterine, intradermal, topical, dermal, parenteral (e.g., subcutaneous, intramuscular, intravenous, intradermal, intraocular, intratracheal and epidural), intranasal, intrathecal, intraarticular, intracavity, transdermal, iontophoretic, ophthalmic, optical (e.g., corneal), intraglandular, intraorgan, and intralymphatic.

As used herein, “therapeutically effective amount” means an amount of a therapeutic compound that is sufficient to significantly induce a physiological response, such as an immune response caused by infection of HIV-1 in an animal, and preferably an owl monkey, or an amount that treats, prevents or emeloriates infection of HIV-1 in an animal, and preferably an owl monkey.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA. Notably, where a nucleotide sequence is provided, the corresponding amino acid sequence is also encompassed within the disclosure and definition. Conversely, where an amino acid sequence is provided, the corresponding nucleotide sequence is also encompassed within the disclosure and definition.

An “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells.

The term “gene” refers to (a) a gene containing a DNA sequence encoding a protein, e.g., CA; (b) any DNA sequence that encodes a protein, e.g., or mutant CA gene amino acid sequence, and/or; (c) any DNA sequence that hybridizes to the complement of the coding sequences of a protein. In certain embodiments, the term includes coding as well as noncoding regions, and preferably includes all sequences necessary for normal gene expression.

As used herein, the term “genome” refers to the HIV-1 genome including all coding, noncoding and regulatory elements.

As used herein a “wild type” means a cell or organism that does not contain the heterologous recombinant DNA that expressed a protein or element that imparts an enhanced trait as described herein.

“Expression” or “expressing” refers to production of a functional product, such as, the generation of an RNA transcript from an introduced construct, an endogenous DNA sequence, or a stably incorporated heterologous DNA sequence. A nucleotide encoding sequence may comprise intervening sequence (e.g., intrans) or may lack such intervening non-translated sequences (e.g., as in cDNA). Expressed genes include those that are transcribed into mRNA and then translated into protein and those that are transcribed into RNA but not translated (for example, siRNA, transfer RNA, and ribosomal RNA). The term may also refer to a polypeptide produced from an mRNA generated from any of the above DNA precursors. Thus, expression of a nucleic acid fragment, such as a gene or a promoter region of a gene, may refer to transcription of the nucleic acid fragment (e.g., transcription resulting in mRNA or other functional RNA) and/or translation of RNA into a precursor or mature protein (polypeptide), or both.

The term “heterologous” refers to a nucleic acid fragment or protein that is foreign to its surroundings. In the context of a nucleic acid fragment, this is typically accomplished by introducing such fragment, derived from one source, into a different host. Heterologous nucleic acid fragments, such as coding sequences that have been inserted into a host organism, are not normally found in the genetic complement of the host organism. As used herein, the term “heterologous” also refers to a nucleic acid fragment derived from the same organism, but which is located in a different, e.g., non-native, location within the genome of this organism. Thus, the organism can have more than the usual number of copy(ies) of such fragment located in its(their) normal position within the genome and in addition, in the case of plant cells, within different genomes within a cell, for example in the nuclear genome and within a plastid or mitochondrial genome as well. A nucleic acid fragment that is heterologous with respect to an organism into which it has been inserted or transferred is sometimes referred to as a “transgene.”

The term, “operably linked,” when used in reference to a regulatory sequence and a coding sequence, means that the regulatory sequence affects the expression of the linked coding sequence.” Regulatory sequences,” or “control elements,” refer to nucleotide sequences that facilitate the transcription of eukaryotic-like mRNAs in prokaryotic cells, and/or facilitate the export of eukaryotic-like mRNAs out of a prokaryotic cells, and/or facilitate the uptake of eukaryotic-like mRNAs by eukaryotic cells, and/or facilitate the translation of eukaryotic-like mRNAs in eukaryotic cells. The terms may additionally encompass nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences and the like. Particular regulatory sequences may be located upstream and/or downstream of a coding sequence operably linked thereto. Also, particular regulatory sequences operably linked to a coding sequence may be located on the associated complementary strand of a double-stranded nucleic acid molecule. As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell.

The term “promoter” or “regulatory element” refers to a region or nucleic acid sequence located upstream or downstream from the start of transcription and which is involved in recognition and binding of RNA polymerase and/or other proteins to initiate transcription of RNA.

An “expression cassette or “expression vector” or “vector” refers to a nucleic acid construct, which when introduced into a host cell, results in transcription and/or translation of a RNA or polypeptide, respectively. More specifically, the term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria. Again, more specifically, “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s). A polynucleotide sequence is operably linked to an expression control sequence(s) (e.g., a promoter and, optionally, an enhancer) when the expression control sequence controls and regulates the transcription and/or translation of that polynucleotide sequence.

The invention encompasses isolated or substantially purified HIV-1 virions or constituents thereof. An “isolated” or “purified” HIV-1 virions or constituents thereof, is substantially or essentially free from components that normally accompany or interact with HIV-1 virions or constituents thereof as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived.

A “variant,” or “isoform,” or “protein variant” is a member of a set of similar proteins that perform the same or similar biological roles. For example, fragments and variants of the disclosed HIV-1 polynucleotides and amino acid sequences encoded thereby are also encompassed by the present invention. By “fragment” is intended a portion of the polynucleotide or a portion of the amino acid sequence. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. Generally, variants of a particular HIV-1 constituent or genome disclosed herein will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

Notably, all peptides disclosed in specifically encompass peptides having conservative amino acid substitutions. As used herein, “conservative amino acid substitutions” means the manifestation that certain amino acids can be substituted for other amino acids in a protein structure without appreciable loss of biochemical or biological activity. Since it is the interactive capacity and nature of a protein that defines that protein's biological functional activity, certain amino acid sequence substitutions can be made in a protein sequence, and, of course, the underlying DNA coding sequence, and nevertheless obtain a protein with like properties. Thus, various changes can be made in the amino acid sequences disclosed herein, or in the corresponding DNA sequences that encode these amino acid sequences, without appreciable loss of their biological utility or activity.

Examples of amino acid groups defined in this manner include: a “charged polar group,” consisting of glutamic acid (Glu), aspartic acid (Asp), asparagine (Asn), glutamine (Gln), lysine (Lys), arginine (Arg) and histidine (His); an “aromatic, or cyclic group,” consisting of proline (Pro), phenylalanine (Phe), tyrosine (Tyr) and tryptophan (Trp); and an “aliphatic group” consisting of glycine (Gly), alanine (Ala), valine (Val), leucine (Leu), isoleucine (Ile), methionine (Met), serine (Ser), threonine (Thr) and cysteine (Cys).

Within each group, subgroups can also be identified, for example, the group of charged polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gin. The aromatic or cyclic group can be sub-divided into the sub-groups consisting of the “nitrogen ring sub-group,” consisting of Pro, His and Trp; and the “phenyl sub-group” consisting of Phe and Tyr. The aliphatic group can be sub-divided into the sub-groups consisting of the “large aliphatic non-polar sub-group,” consisting of Val, Leu and Ile; the “aliphatic slightly-polar sub-group,” consisting of Met, Ser, Thr and Cys; and the “small-residue sub-group,” consisting of Gly and Ala. Examples of conservative mutations include substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gin for Asn such that a free —NH2 can be maintained.

Proteins and peptides biologically functionally equivalent to the proteins and peptides disclosed herein include amino acid sequences containing conservative amino acid changes in the fundamental amino acid sequence. In such amino acid sequences, one or more amino acids in the fundamental sequence can be substituted, for example, with another amino acid(s), the charge and polarity of which is similar to that of the native amino acid, i.e., a conservative amino acid substitution, resulting in a silent change. It should be noted that there are a number of different classification systems in the art that have been developed to describe the interchangeability of amino acids for one another within peptides, polypeptides, and proteins. The following discussion is merely illustrative of some of these systems, and the present disclosure encompasses any of the “conservative” amino acid changes that would be apparent to one of ordinary skill in the art of peptide, polypeptide, and protein chemistry from any of these different systems. Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. Table 13, infra, contains information about which nucleic acid codons encode which amino acids.

### Amino Acid Nucleic Acid Codons

| Amino Acid | Nucleic Acid Codons |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

As used herein and in the appended claims, the singular forms “a”, “an”, and “the” include plural referents unless the context clearly dictates otherwise. Thus, for example, reference to “a plant” includes a plurality of such plants; reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or +a range of up to 20%, up to 15%, up to 10%, up to 5%, or up to 4%, 3%, 2%, or 1% compared to the specifically recited value.

The term “comprising” as used in a claim herein is open-ended, and means that the claim must have all the features specifically recited therein, but that there is no bar on additional features that are not recited being present as well. The term “comprising” leaves the claim open for the inclusion of unspecified ingredients even in major amounts. The term “consisting essentially of” in a claim means that the invention necessarily includes the listed ingredients, and is open to unlisted ingredients that do not materially affect the basic and novel properties of the invention. A “consisting essentially of” claim occupies a middle ground between closed claims that are written in a closed “consisting of” format and fully open claims that are drafted in a “comprising′ format”. These terms can be used interchangeably herein if, and when, this may become necessary. Furthermore, the use of the term “including”, as well as other related forms, such as “includes” and “included”, is not limiting. Notably, where the specification or other parts of this application refer to a polynucleotide sequence, it may also refer to the corresponding protein sequence and vice-verse.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Background, Experimental Overview, and Rationale

HIV/AIDS is an ongoing pandemic that won't end until vaccines are developed. The key to the chronic and deadly nature of HIV-1 is that the virus sets up a reservoir of infected cells in the body. HIV-1 stably integrates into the genome of target cells, and these cells then divide and sustain a population of cells capable of producing infections HIV-1. Thus, it is particularly important that HIV-1 vaccines induce effective non-neutralizing antibodies and T cells because these mechanisms kill infected cells. Conversely, an effective HIV-1 vaccine will likely not be based on the surface protein of HIV-1 alone (Envelope, Env), because Env-based immunogens predominantly induce neutralizing antibodies which can block free virus but do not kill infected cells.

As noted above, a major limitation in the development of an HIV vaccine has been the current vaccine challenge model, SHIV infection of macaques, which only allows the testing of Env immunogens. To develop an effective HIV-1 vaccine, different HIV-1 immunogens beyond Env and how to deliver are required. Because epitopes in every HIV-1 protein can theoretically induce unique non-neutralizing antibodies and T cells, it will be important that vaccines contain as immunogens as many HIV-1 proteins as possible. To properly test the breadth of immune responses induced by candidate vaccines, the challenge virus must also contain most or all of the HIV-1 genome (and therefore most or all of the epitopes).

Others in the field have attempted to produce a suitable animal model for HIV vaccine development. For example, prior research has developed a single model of a primate infected with minimally modified HIV-1. The virus was initially designed to have a substituted Vif and was used to infect pigtailed macaques, where the virus evolved compatibility to pigtail macaque CD4, Tetherin, and MX2 (15, 26). Chronic CD8+ T cell depletion in monkeys was required for that virus to reach high titers and evolve compatibility with pigtailed macaque. After over 4 years of adaptation and passage, a pathogenic clone was isolated. One potential concern is that this virus has been evolved in the absence of CD8+ cytotoxic T cells, which would presumably make it hyper-sensitive to vaccine-induced T cell immunity. To date, this virus in pigtailed macaque has not been demonstrated as a vaccine model.

Here, Applicants report the development of this needed primate model for HIV-1. Fully immune competent owl monkeys are readily infected with HIV-1 (93% wildtype in sequence) administered intravenously as a purified virus or through blood transfusions from previously infected animals. Owl monkey infections recapitulate key features of human HIV-1 infections: an acute phase of infection with plasma viremia up to 10⁷ copies/mL, subsequent control of the virus, seroconversion, and the establishment of a small virus reservoir from which virus rebound occurs. Thus, the owl monkey model represents the long-sought pre-clinical testing ground for HIV-1 vaccines. Specifically, Applicant's invention allows vaccines tested in owl monkeys to be assessed for their ability to: 1) block virus replication during the acute phase, 2) clear virus from the body more quickly, and 3) block formation of a reservoir as measured by dexamethasone-induced virus rebound in subsequent weeks and months.

As detailed below, in one embodiment HIVom1* is an improvement over the existing HIV-1 vaccine model that employs SHIVs. SHIVs are SIVmac strains that have had the region around Env replaced with the HIV-1 genome. Even the most state-of-the art SHIVs only have ˜30% of their genome derived from HIV-1. To the contrary, Applicant's engineered virus is 93% wildtype HIV-1 in sequence and therefore will contain 93% of the HIV-1 epitopes relevant to adaptive immune responses, compared to SHIVs which will contain ˜30%. Moreover, SHIVs must be adapted such that they acquire mutations in Env that allow it to use the macaque CD4 which is non-permissive for HIV-1 entry. Applicants have shown that these Env adaptations alter the biology of HIV-1 in ways that are not completely understood. In contrast, owl monkey CD4 behaves like human CD4 and is not a barrier for most strains of HIV-1.

### Example 2: Determining Immune Bypasses Required for Host Switching of HIV-1 into Owl Monkeys

It was determined early in the HIV/AIDS pandemic that unmodified HIV-1 does not infect monkeys of any species. To identify research species with minimal genetic barriers to HIV-1, and because monkeys are not isogenic, Applicants obtained blood draws from over a thousand monkeys representing 14 research species (data not shown). From each, Applicants made cDNA libraries and sequenced transcripts of genes encoding the HIV-1 receptor CD4 and three powerful restriction factors of HIV-1: Tetherin, TRIM5α, and APOBEC3G. These four genes are evolving under intense positive natural selection in primates, resulting in highly divergent proteins across and within species. Applicants sequenced the two alleles of each gene present in each animal and made molecular clones of each allele that Applicants identified. Each allele was then tested in vitro for the ability to support entry of HIV-1 (CD4), or restrict HIV-1 (Tetherin, TRIM5α, and APOBEC3G) (data not shown).

Applicants previously found that the CD4 orthologs of several owl monkey species (Aotus spp.) support entry of HIV-1, unlike the CD4 orthologs of most other primate species, including macaques. Applicants had access to a large colony of captive Nancy Ma's owl monkeys (Aotus nancymaae), from which Applicants took blood samples from 191 individuals. In owl monkeys, Applicants found alleles encoding 18 distinct CD4 variants (FIG. S1, Align. S1). Applicants cloned, expressed, and tested the most common ones, all of which supported HIV-1 entry (FIG. S2). Tetherin interferes with HIV-1 budding by tethering virions to the cell membrane, thereby limiting their release. In Nancy Ma's owl monkeys, Applicants found alleles encoding eight distinct Tetherin variants (FIG. S1, Align. S2), none of which inhibited HIV-1 virion release (FIG. S3) consistent with previous findings for the Tetherin homolog of another owl monkey species. Therefore, Nancy Ma's owl monkeys naturally have two critical genetic advantages as a possible HIV-1 model because both their CD4 and Tetherin homologs are compatible with HIV-1 infection.

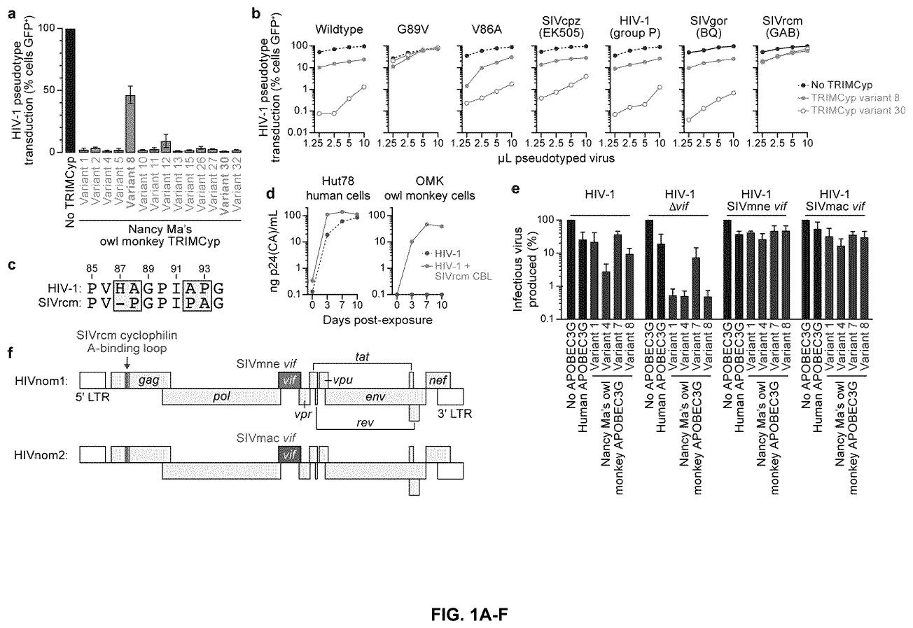

TRIM5α evolved into “TRIMCyp” in owl monkeys. TRIMCyp targets and disrupts the HIV-1 capsid protein p24 (CA) during virion entry. Applicants found 37 unique TRIMCyp protein variants in the captive animals (FIG. S1, Align. S3), and with one exception (variant 8) these potently inhibit HIV-1 (FIG. 1a). As shown before, a G89V substitution in the cyclophilin A-binding loop of HIV-1 p24(CA) enabled escape from owl monkey TRIMCyp restriction (FIG. 1b). However, Applicants found that the G89V change reduced viral fitness (FIG. S4), and Applicants were unable to obtain significant spreading infection with this mutation in the context of our viruses. Consequently, Applicants sought to engineer HIV-1 to escape owl monkey TRIMCyp in a different manner by using variation in lentiviral capsid proteins found in nature. Applicants systematically substituted the 10-amino-acid-long HIV-1 cyclophilin A-binding loop with those from various SIV and HIV-1 strains. Applicants identified one, from red-capped mangabey SIV (SIVrcm), that differed from that of HIV-1 in only four positions (FIG. 1c, FIG. S5) yet enabled HIV-1 to bypass owl monkey TRIMCyp restriction (FIG. 1b). HIV-1 with just these four mutations replicated robustly in OMK (owl monkey kidney) cells, overcoming the powerful inhibition of TRIMCyp otherwise observed in these cells (FIG. 1d).

Finally, Applicants investigated the activity of APOBEC3G, a restriction factor that causes hypermutation of reverse transcribed HIV-1 genomes unless it is degraded by the HIV-1-encoded virion infectivity factor (Vif). APOBEC3G is not expressed in OMK cells but is expected to inhibit HIV-1 in owl monkey T cells. Applicants identified alleles encoding 14 protein variants of APOBEC3G in the captive animals (FIG. S1, Align. S4). At least one of the four most common variants inhibits HIV-1 replication, even in the presence of HIV-1 Vif (FIG. 1e). Consequently, Applicants tested 42 different HIV-1 and SIV Vifs and identified several that degrade owl monkey APOBEC3G variants (FIGS. S6, S7). HIV-1 engineered to encode Vif from either SIVmne (pigtailed macaque) or SIVmac (rhesus macaque) bypassed restriction by all tested owl monkey APOBEC3G variants (FIG. 1e).

To produce an HIV-1 strain potentially capable of infecting owl monkeys, Applicants modified a plasmid encoding the full-length HIV-1 genome (strain NL4-3 (SEQ ID NO. 1)) to introduce the four point mutations reconstituting the SIVrem cyclophilin A-binding loop (FIG. 1c) and either SIVmne or SIVmac Vif. Applicants named these two viruses HIVnom1 and HIVnom2, respectively, following lentiviral naming conventions in which the subscript refers to Nancy Ma's owl monkey (FIG. 1f).

### Example 3: HIV-1 Infection of Owl Monkeys

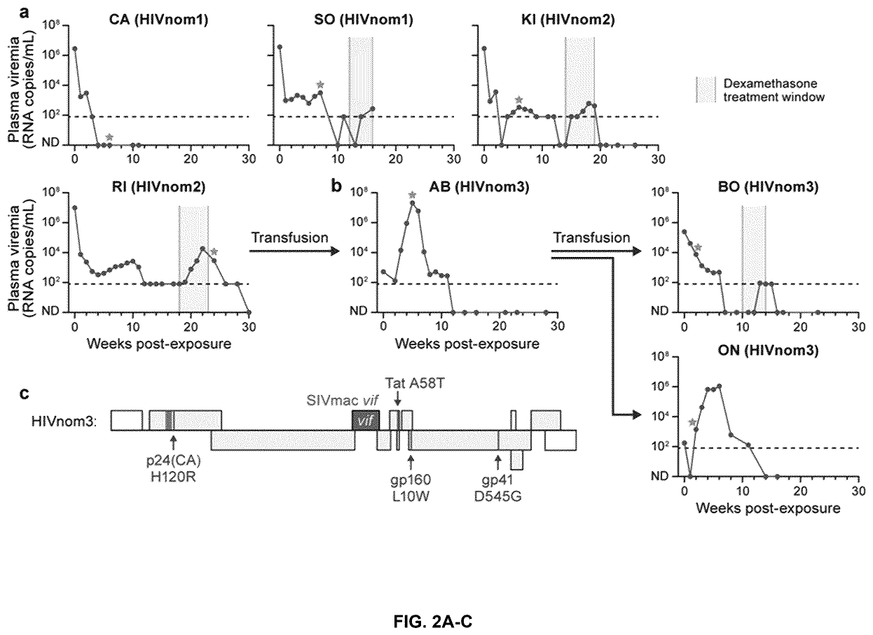

Two Nancy Ma's owl monkeys were intravenously injected with 1.3×10⁶ infectious units of HIVnom1 and another two with 2.1×10⁶ infectious units of HIVnom2 (FIG. 2a). Blood was drawn weekly for up to 30 weeks, with the “0” timepoint taken from the leg opposite to the site of virus injection five minutes after inoculation. Blood samples were sent to the University of Washington Clinical Retrovirology Laboratory (a human clinical diagnostics lab in Seattle, WA, USA) for determination of plasma viremia by RT-qPCR. Although virus replication in these first four animals was modest, in each animal there was one or more timepoint at which plasma viremia increased, suggesting that HIV-1 progeny virions were being produced (FIG. 2a). By week 12, viremia in all animals was reduced to a level at or below the assay limit of quantification (dotted line; 80 HIV genomes per mL). As such, it was unclear whether the animals had successfully cleared the virus and eliminated it from their bodies. To test this, Applicants subcutaneously administered dexamethasone over a course of several weeks to three of the four animals. Dexamethasone, a glucocorticoid, induces transient immunosuppression by reversibly depleting blood lymphocytes, NK cells, and B cells, but not monocytes, in people and owl monkeys (FIG. S8). In all three animals, virus rebounded into the blood to detectable levels during treatment (FIG. 2a). These data demonstrate that virus persists in owl monkeys, and that there is immune pressure being exerted on the virus that is suppressing viremia. Together, these data demonstrate the replication of close-to-wildtype HIV-1 in a fully immunocompetent small nonhuman primate host, including establishment of a viral reservoir.

Of the four infected animals, animal RI (infected with HIVnom2) had the highest and longest acute phase (0-10 week) viremia, and the highest rebound viremia upon dexamethasone treatment (FIG. 2a). Frozen plasma collected from animal RI at weeks 21 and 22 was combined and transfused into a naïve animal (AB). At this point, Applicants observed a dramatically higher level of replication. Plasma viremia of AB five minutes after transfusion was 526 HIV-1 genomes per mL but, within 5 weeks, expanded by five orders of magnitude to 2.1×10⁷ HIV-1 genomes per mL (FIG. 2b). Sequencing of integrated viral genomes from the PBMCs of animal AB revealed that HIVnom2 had acquired four fixed nonsynonymous point mutations that were not present in the HIVnom2 stock used for infection of source animal RI. This virus, here referred to as HIVnom3, is characterized by four new amino-acid substitutions: in p24 (CA) (H120R), Tat (A58T), and Env (L10W in the signal peptide; D545G in the gp41 subunit) (FIG. 2c). Three of the mutations arose in animal RI, with the final mutation arising in animal AB (FIG. S9). Notably, three of these four substitutions are to amino acids commonly circulating in transmitted forms of HIV-1 (FIGS. S10-S13), preserving the likeness to HIV-1. Further, these four substitutions did not cause an obvious fitness loss in human T cells (FIG. S14). The four mutations remained fixed in all subsequent infections, and no additional fixed HIV-1 mutations were ever detected in any subsequent experiments.

Frozen plasma and fresh whole blood collected from animal AB at weeks 4 and 5, respectively, were mixed and transfused into one naive animal (BO). Likewise, frozen plasma and fresh whole blood collected from animal AB at weeks 7 and 8, respectively, were mixed and transfused into a second naive animal (ON) (FIG. 2b). In BO, replication was modest and then decreased over time until no longer detectable. To ensure that virus persisted in the animal, Applicants waited for one month after viremia was last detected and then treated the animal with dexamethasone upon which virus rebounded to low detectable levels (FIG. 2b). In contrast, a significant acute viral peak was observed in the second monkey, ON. The plasma viremia of ON five minutes after transfusion was 175 HIV-1 genomes per mL but, within six weeks, expanded by four orders of magnitude to 1.1×10⁶ HIV-1 genomes per mL (FIG. 2b). Applicants conclude that HIVnom3 is replicating in owl monkeys: it can propagate to high titers during the acute phase of infection in at least some owl monkeys, causes animals to seroconvert (yellow stars in FIG. 2a,b), can rebound upon dexamethasone-induced immunosuppression, and can be transmitted from one animal to the next through infected blood products.

### Example 4: HIVnom3 is an Owl Monkey-Adapted Virus

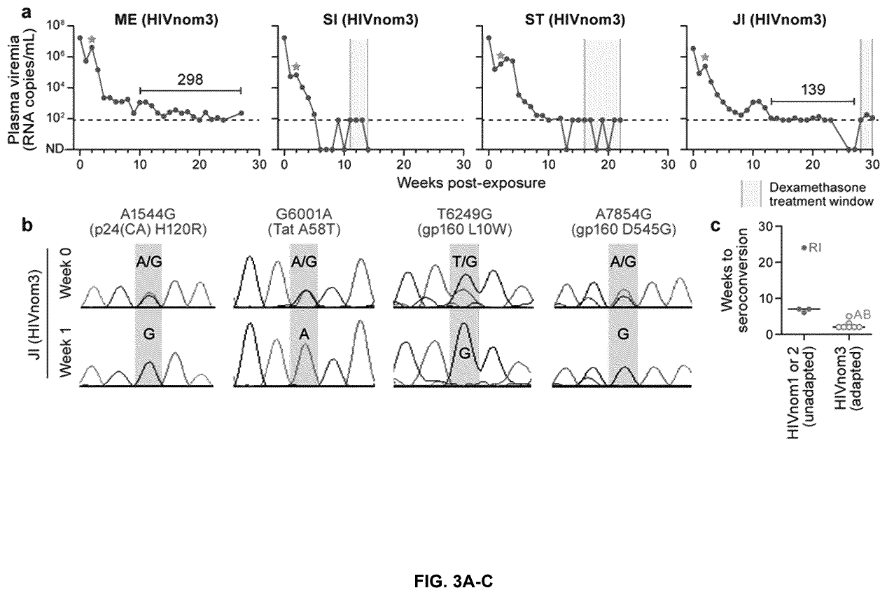

Applicants speculated that HIVnom3 may have adapted to the owl monkey host during the serial passage experiment. To test this hypothesis, Applicants introduced the four HIVnom3-specific mutations into the plasmid-based molecular clone of HIVnom2 (FIG. 2c). Both plasmids were then used to create virus stocks. A 50:50 mixture of the original HIVnom2 and HIVnom3 (which differ by only four point mutations) was intravenously injected into two male and two female owl monkeys of varying ages. The goal was to determine whether one virus would outcompete the other in vivo. In all four animals, the viruses replicated and, whenever tested, rebounded upon dexamethasone treatment (FIG. 3a). HIVnom3 proved to be more fit that HIVnom2, which could no longer be detected at the 1-week timepoint in any of the four animals (FIG. 3b, FIGS. S15-S18).

Increased fitness of HIVnom3 is supported by two additional observations. First, all four animals infected with HIVnom3 seroconverted more rapidly than animals infected with HIVnom1 or HIVnom2 (FIG. 3c). Times to seroconversion were correlated with the presence of the four new mutations but not with the dose of inoculated viruses (FIG. S19). Second, HIVnom3 set point viremias in two monkeys (ME: average 298 RNA copies/mL and JI: average 139 RNA copies/mL) were higher than set points observed in any previous experiment, which tended to be below the limit of quantification except when dexamethasone was administered. Sequencing of proviruses in PBMCs collected from these two animals (ME and JI) at necropsy yielded the sequence of HIVnom3 and did not reveal any additional fixed virus mutations. Collectively, these data indicate that the four mutations acquired during in vivo serial passage are sufficient for adapting HIV-1 to owl monkeys. Stocks of this virus can be created from the HIVnom3 molecular clone, ensuring genetic stability of this owl monkey-adapted virus over time.

### Example 5: Immunological and Histological Responses to Infection in Owl Monkeys

Applicants next investigated antibody dynamics in HIVnom-infected owl monkeys. All animals infected in this study seroconverted, developing antibodies to HIV-1 gp41, gp160, and/or p24 (CA) (FIG. 4a; antibodies to other HIV-1 antigens were not tested). With in vitro experiments, Applicants showed that HIVnom3 is effectively neutralized by commonly studied neutralizing monoclonal antibodies (FIG. S20). All investigated animals also developed autologous neutralizing antibodies except for animal ON (FIG. 4b, FIG. S21).

Physical examinations with complete blood counts, serum chemistry analyses, and flow cytometric analyses of blood leukocyte populations were performed periodically on the animals throughout the duration of the study. Each animal also underwent a complete necropsy with histological examination of at least 44 tissues upon study termination. For the duration of these experiments (˜7 months per animal), there were no clinical abnormalities, laboratory changes, gross necropsy findings, or histological lesions identified that could be directly attributable to HIV-1/HIVnom infection or simian AIDS.

In people, HIV-1 proviruses in lymph nodes continue to produce viral transcripts even during full suppression of viremia. Limited access of cytotoxic T cells to lymph nodes is thought to contribute to this persistent viral replication at this site. Applicants therefore investigated whether HIVnom3 was active in lymph nodes in infected owl monkeys. RNAscope in situ hybridization was performed on formalin-fixed lymph node sections using an HIV-1-specific probe set recognizing transcribed copies of the HIV-1 genome. In positive control lymph node samples from a person with HIV-1, Applicants observed only rare cells transcribing HIV-1 RNA (FIG. 4c). In people, as few as ˜12 per 10⁶ CD4⁺ T cells in lymph nodes are actively transcribing HIV-1 RNA. Applicants then examined lymph nodes from different anatomical locations in six owl monkeys infected with HIVnom3 (FIG. 4d). Like in the human samples, Applicants identified low numbers of HIVnom3 RNA-positive cells in all examined owl monkey samples (FIG. 4d-g). No staining was identified in pancreas of infected animals, or in lymph nodes of naive animals (FIGS. 4h,i, S22, S23). Four of these six animals (AB, ON, ME, SI) were non-viremic at the time of necropsy yet were transcribing HIVnom3 genomes within cells of their lymph nodes. Animal AB is particularly noteworthy here as this animal had been non-viremic for 37 weeks (≈9 months) at necropsy, yet HIVnom3 RNA was clearly being transcribed within the lymph nodes (FIG. 4e). These RNAscope studies, in combination with rebound of plasma viremia upon dexamethasone treatment, demonstrate the presence of an active virus reservoir in lymph nodes and possibly in other locations as well.

### Example 6: Results Overview and Summary

Animal models for vaccine development, in particular, should recapitulate key events of the first days to weeks of human infection. This is because years of post-exposure treatment (PEP) has demonstrated that HIV-1 becomes permanently established within three days of exposure. Thus, the timeline for executing vaccine-induced immunity to fight off the virus is short, and a successful HIV-1 vaccine will have to provide protection within the first days to weeks after exposure. There are several key hallmarks of early HIV-1 infection in people, which are recapitulated in the Nancy Ma's owl monkey model.

First, people with HIV-1 demonstrate an acute phase burst of virus replication within the initial 10 weeks of infection. All infected owl monkeys had at least modest increases in plasma viremia during this phase and some had profound acute phase virus bursts (FIGS. 2, 3). Acute viral peaks in animals AB and ON reached 2.1×10⁷ and 1.1×10⁶ HIV-1 genomes per mL, like the median acute viral peak in people of 5.0×10⁶ HIV-1 genomes per mL (range: 3.2×10⁴-3.2×10⁸). In other animals, virus replication was more modest, but initial replication may have also been masked by the large inoculating bolus.

Second, people with HIV-1 produce anti-HIV-1 antibodies (i.e., seroconvert) by a median of two weeks after first testing positive for viral RNA (range: 1-7 weeks). All infected owl monkeys seroconverted within this time range except for animal RI, which needed 24 weeks (FIG. 3c, 4a, S19). Like in most people, most infected animals also developed autologous neutralizing antibodies (FIG. 4b, S21).

Third, after the acute phase, plasma viremia in people stabilizes at a low level referred to as the “set point” viremia. In untreated people, the median set point is 2.5×10⁴ HIV-1 genomes per mL (range: 320-1.0×10⁶). Owl monkeys also reduce viremia by 10 weeks, but they do so more profoundly than do most untreated people. The set point is often detectable but below the limit of quantification, similar to human elite controllers. After full adaptation of HIVnom3, some monkeys demonstrated higher viral set points, in the range of 100-300 HIV-1 genomes per mL (FIG. 3a).

Fourth, once infected, people never clear HIV-1 from their body. A persistent reservoir of HIV-1-infected cells is established in lymph nodes and other locations. Applicants observed HIVnom3 genome transcription in the lymph nodes of all investigated animals, in one case 37 weeks after virus was cleared from the bloodstream (FIG. 4d-g, S22). Further, in all animals tested, dexamethasone treatment led to virus rebound into the bloodstream, at least to detectable levels (FIGS. 2, 3). These findings demonstrate the presence of an HIVnom reservoir in lymph nodes and possibly other locations. Further work is needed to understand the cell types involved in this reservoir.

Accurate recapitulation of the virus reservoir is of particular interest, because vaccines will need to inhibit formation of this reservoir. Some people are thought to develop an exceedingly small reservoir. In persons who are treated with antiretrovirals early in infection, rebound viremia is very low (<100 HIV-1 genomes per mL) and takes weeks to months to occur. Likewise, the reservoir in owl monkeys appears to be small. Rebound viremia in owl monkeys upon treatment with dexamethasone was low, with peak viremias of 276 (animal SO), 612 (animal KI), 18,220 (animal RI), 92 (animal BO), and 258 (animal JI) HIVnom genomes per mL (FIGS. 2, 3). In animals SI and ST, viral RNA was detected during dexamethasone treatment, but concentrations never surpassed the limit of quantification (FIG. 3). Also supporting a small reservoir is the very low percentage of cells in the monkeys' lymph nodes that are producing HIVnom3 RNA (FIGS. 4e-g, S22), as is also observed in human lymph nodes (FIG. 4c, S23). Additional experiments characterizing the viral reservoir in owl monkeys are underway.

Based on these data the owl monkey model provides a complement vaccine development efforts. SHIVs are adequate challenge viruses for evaluating neutralizing antibody responses to Env-based vaccines, because Env in SHIVs derives from HIV-1. However, SHIVs are inappropriate challenge viruses for evaluating vaccines containing most other HIV-1 immunogens, which in SHIVs derive from the simian virus, SIV. Vaccines can induce target-specific neutralizing antibodies, non-neutralizing effector antibodies, and target-specific CD8+ T cells. Decades of research in humans, rodent models, and macaque models have shown that these responses will likely all be important in a successful vaccine response. Because epitopes throughout the HIV-1 genome can be targets of cytotoxic CD8+ T cells, challenge viruses in vaccine studies need to contain as many of these epitopes as possible. SHIVs are about 30% HIV-1 in their genome composition, whereas the HIVnom3 genome is 93% derived from HIV-1. Thus, HIVnom3 contains more of the relevant epitopes for cell-mediated immune responses than do SHIVs. Consequently, Applicants anticipate that HIVnom3 is valuable challenge virus that complements SHIVs, particularly for studies aimed at clearing HIV-1-infected cells. Notably, this is a small monkey with a relatively fast breeding time. Owl monkeys have a decades-long history as a malaria vaccine model (5, 6), and so there are significant research tools available.

### Example 7: HIVnom3 Virus Used in the Owl Monkey Model Recapitulates Key Hallmarks of Human HIV-1 Infection

As noted above, it was determined early in the HIV/AIDS pandemic that unmodified HIV-1 does not infect monkeys of any species. To identify research species with minimal genetic barriers to HIV-1, and because monkeys are not isogenic, Applicants obtained blood draws from over a thousand monkeys representing 14 research species. From each, we made cDNA libraries and sequenced transcripts of genes encoding the HIV-1 receptor CD4 and three powerful restriction factors of HIV-1: Tetherin, TRIM5α, and APOBEC3G. These four genes are evolving under intense positive natural selection in primates, resulting in highly divergent proteins across and within species. Applicants sequenced the two alleles of each gene present in each animal and made molecular clones of each unique allele that was identified. Each allele was then tested in vitro for the ability to support entry of HIV-1 (CD4), or restrict HIV-1 (Tetherin, TRIM5α, and APOBEC3G). As a result of this work, Applicants have successfully infected 25 owl monkeys with an owl monkey adapted HIV-1. Each of the 25 animals was followed for 4-12 months, generating the following supplemental data.

The HIVnom3 virus used in the owl monkey model: HIVnom3 (“nom” stands for Nancy ma's owl monkey) a/k/a ATCC Patent Deposit No. PTA-127573 (See Section on Naming Convention). The virus is based on HIV-1 subtype B isolate NL4-3. Applicants engineered this virus to contain 8 point mutations and a replacement of the vif gene with the vif of SIVmac (FIG. S26). Five of the point mutations are in and near the cyclophilin binding loop of p24 (CA) and provide escape from owl monkey TRIM-Cyp (their version of TRIM5α). Vif from SIVmac allows bypass of owl monkey APOBEC3G. The other 3 point mutations arose during serial passage in owl monkeys, and result in substitutions to amino acids common in T/F strains of HIV-1, preserving the likeness to HIV-1. HIVnom3 is 93% identical to the original NL4-3 sequence. Virus stocks are generated from a molecular clone, ensuring genetic stability of the virus over time.

Owl monkey model recapitulates key hallmarks of human HIV-1 infection: Humans demonstrate an acute phase burst of virus replication within the initial 10 weeks of infection. HIVnom3 infected owl monkeys show this as well (FIG. S27). Acute viral peaks in owl monkeys commonly reach 10⁶ HIV-1 genomes per mL, like the median acute viral peak in humans of 5.0×10⁶ HIV-1 genomes per mL (range: 3.2×10⁴-3.2×10⁸). Applicants have determined the minimal infectious dose of HIVnom3 to be 10,000 infectious virions, a dose at which we have observed 100% of owl monkeys to become infected (FIG. S27).

After the first 10 weeks, plasma viremia in humans stabilizes at a lower level referred to as the viral “set point.” In untreated humans, the median set point is 2.5×10⁴ HIV-1 genomes per mL. Owl monkeys also reduce viremia after the acute phase, with a set point that is typically 1×10²-1×10³ HIV-1 genomes per mL (FIG. S27). This set point is lower than typically seen in untreated humans, and more like HIV “controllers.” In humans, HIV controllers are those that sustain set points <2,000 HIV-1 genomes per mL. (Human elite controllers are, further, those that sustain set points <50 copies/mL.)

Once infected, a persistent reservoir of HIV-1-infected cells is established in human lymph nodes and other locations. In humans, HIV-1 proviruses in some infected cells in lymph nodes continue to produce viral transcripts even during full suppression of viremia. In humans, as few as ˜12 per 10⁶ CD4⁺ T cells in lymph nodes are actively transcribing HIV-1 RNA. Applicants observe similar HIVnom3 genome transcription in the lymph nodes of infected owl monkeys (FIG. S28). Animal AB is particularly noteworthy here, as this animal had been non-viremic for 37 weeks (˜9 months) at necropsy, yet HIVnom3 RNA was clearly being transcribed within the lymph nodes. These findings of HIVnom3 transcription in lymph nodes during suppressed viremia, and dexamethasone-induced rebound discussed below, suggest the presence of an HIVnom3 reservoir in lymph nodes and possibly other locations. Finally, as in humans, HIVnom3 is transmissible between owl monkeys by needle injection and transfusion of blood products.

Preliminary evidence of T cell and other immune responses in the owl monkey: HIVnom3 transcription in lymph nodes during suppressed viremia (FIG. S28) suggests some level of immune control in the owl monkey. There are other data to support this. In a few animals, Applicants observe undetectable viremia after the acute phase, like elite controllers (one such animal shown in FIG. S29A). Set point viremia in these animals is below 80 HIV-1 RNA copies per mL, our assay LoQ. To determine the role of immunity in this control, Applicants administered dexamethasone over a course of several weeks. Dexamethasone, a glucocorticoid, induces transient depletion of blood lymphocytes (FIG. S29A, bottom) and other immune cells in humans and owl monkeys. During dexamethasone treatment, viremia rebounds to detectable levels (FIG. S29A, top). This demonstrates that viremia is being actively suppressed by immune regulation.

Preliminary evidence for T cell responses: Humans seroconvert by 7 weeks, and infected owl monkeys also seroconvert within this time range. Most infected owl monkeys also develop autologous neutralizing antibodies (FIG. S29B). Since we see neutralizing antibodies, CD4+ T cells are likely active in the response to infection. CD4+ T cells, also known as helper T cells, are integral in coordinating and maturing antibody responses by delivering essential signals to B cells. Further, in humans with HIV-1, initial replication occurs in the gut-associated lymphoid tissue (GALT) with quantifiable reduction of CD4+ T cell frequencies. Although to date we have only performed a limited number of GALT surgeries at early infection timepoints in owl monkeys, we do see the initial indication of CD4+ T cell reduction in GALT (FIG. S29C). Finally, in humans, HIV-1-specific CD8+ T cells are linked to the reduction of viremia during the acute phase. Notably, a surge of CD8+ T cells is observed in infected owl monkeys correlating with this reduction in viremia (FIG. S29D).

Demonstration of the owl monkey as a model of HIV-1 protection: As noted above, Applicants have also shown that owl monkeys can model protection, as required in a vaccine model. In a passive protection study, Applicants administered to animals a single intravenous 40 mg/kg dose of a monoclonal neutralizing Ab (3BNC117) or an isotype control. Twenty-four hours later, each monkey was injected with 10,000 infectious units of HIVnom3, and plasma viremia was measured weekly (FIG. S30A). The single dose of neutralizing Ab elongated the time to peak viremia (FIG. S30B) and reduced that peak viremia when it did occur (FIG. S30C). The virus reservoir was still clearly established because virus rebounded as the nAb concentration diminished over time (FIG. S30A, right graph).

Antibodies for flow cytometry analysis of owl monkey immune cells: Many commercially available antibodies directed at human proteins cross-react with owl monkey proteins. This is because human and owl monkey proteins are, on average, about 87% identical in amino acid sequence. Applicants have identified available antibodies that detect the following owl monkey proteins by flow cytometry: CD3, CD20, CD16, CD4, CD8, CD14, CCR5, CXCR4, CD45RA, CCR7, CD95, HLA-DR. As an example, FIG. S31 shows Applicants gating strategy for HIV-1 target cells within mononuclear cells isolated from a GALT biopsy.

Ex vivo proliferation of CD4+ and CD8+ T cells from naive owl monkeys: For some experiments, ex vivo proliferation and maintenance of T cells from owl monkey PMBCs can be desired. T cells require external signals for survival and proliferation. For human T cell proliferation, phytohemagglutinin (PHA) and concanavalin A (Con A) are the primary mitogens used. Human IL-2 is also known to trigger a signaling cascade that promotes survival, proliferation, and differentiation of human T cells. Prior work has shown that these mitogens, as well as human IL-2, also promote the growth of owl monkey T cells in vitro. Indeed, Applicants can proliferate CD4+ T cells (FIG. S32A) and CD8+ T cells (FIG. S32B) from owl monkey PBMCs, to similar levels as from human PBMCs, using IL-2 and PHA. T cells stimulated in this manner can be productively infected with HIVnom3 (FIG. S32C) generating target cells for assays of CD8+ T cell-mediated killing.

Custom antibody for in vivo depletion of owl monkey CD8+ T cells: Applicants sequenced the IgG Fc constant region of the owl monkey (80% identical to human) and made an αCD8 alpha antibody that transiently depletes CD8+ T cells in owl monkeys. After a single 50 mg dose of the antibody, administered by intravenous slow infusion, CD8+ T cells go to nearly undetectable levels and stay suppressed for several months (FIG. S33A).

ELISPOT assay for owl monkeys: The activation of PBMCs can be measured in multiple ways, but one of the most common is the ELISPOT assay. Applicants have verified the Mabtech IFN-γ ELISPOT kit (ELISpot Pro: Monkey IFN-γ (ALP)) for use with owl monkey PBMC samples (FIG. S33 B,C). ELISPOT assays measuring perforin have previously been performed with owl monkey PBMCs.

### Example 8: Materials and Methods

### Nonhuman Primates and Biological Materials

Nancy Ma's owl monkeys (Aotus nancymaae) and blood samples used in this study originated from a breeding colony housed at the MD Anderson Cancer Center, Keeling Center for Comparative Medicine and Research (KCCMR) located in Bastrop, TX, USA. Naïve colony animals were socially housed in environmentally controlled indoor enclosures containing nest boxes, perches, and a variety of physical enrichment manipulanda. Commercial monkey chow and water were available ad libitum, and fresh produce and novel food enrichment items were also provided daily. The KCCMR is accredited by the Association for the Assessment and Accreditation of Laboratory Animal Care International. All animal work was approved by The University of Texas, MD Anderson Cancer Center Institutional Animal Care and Use Committee (IACUC Protocol #s: 00001839-RN00, 00001839-RN00, 00000451-RN02, and 00000451-RN03) and was performed in accordance with the standards established by the Guide for the Care and Use of Laboratory Animals of the National Institute of Health, the Office of Animal Welfare, and the United States Department of Agriculture. Experimental virus exposures of these animals are described below. Other blood collections and clinical procedures (e.g., physical exams, dexamethasone administration, potassium administration) were performed on animals as they were briefly manually restrained, without the use of sedation.

### Genetic Characterization of Nancy Ma's Owl Monkeys

For genetic characterization of the KCCMR colony, 2.5 mL of blood were collected from naïve colony animals in RNA preservation tubes (PAXgene Blood RNA Tube, BD Biosciences #762165) that were processed according to the manufacturer's instructions. These animals were immediately returned to the breeding colony following collection. Individuals were surveyed for genetic diversity at four genetic loci: CD4, tetherin, TRIMCyp, and APOBEC3G. Blood samples were collected from 191 naïve owl monkeys. RNA was isolated using TRIzol (Invitrogen #15596018) and cDNA libraries were prepared using the SuperScript IV First-Strand Synthesis System (Invitrogen #18091200). Gene-specific primers (placed in the 5′ and 3′ UTRs) were then used to PCR-amplify nucleic acids encoding CD4, tetherin, TRIMCyp, and APOBEC3G from cDNA using Q5 High-Fidelity DNA Polymerase (New England BioLabs #M0491L). All PCR products were treated with Exonuclease I (Thermo Fisher Scientific #70073) and recombinant Shrimp Alkaline Phosphatase (rSAP, Thermo Fisher Scientific #78390), and then quantified using the EZQuant dsDNA Quantitation Kit (Fluorometric, BioVision #K900-2000). These products were pooled in equimolar quantities by individual, and then barcoded before being sequenced using a MiSeq instrument (Illumina). Raw sequenced reads were processed as follows: Adapters were trimmed using Trimmomatic v0.36⁵¹ generating both paired-end and unpaired trimmed reads. FastQC v0.11.7⁵² was used to assess the quality of the trimmed raw sequences. Sequencing reads were re-assembled into the four targeted genes using GenBank reference sequences CD4 (#XM_021670978), tetherin (#XM_021669213), TRIMCyp (#XM_012456799), and APOBEC3G (#NM_001308530). An indexed reference sequence was created utilizing BWA v0.7.17⁵³ and Samtools v1.6⁵⁴. Mapping the trimmed, quality-controlled reads to the indexed reference sequence was accomplished using BWA-MEM v0.7.17⁵⁵ to create SAM files. The conversion of SAM to the binary BAM format, subsequent sorting, and removal of duplicate reads were performed using Picard Tools v2.6.0 (https://broadinstitute.github.io/picard; Broad Institute). VCF files containing genotype likelihoods were generated from the marked BAM files with the mpileup algorithm in BCFtools v1.8⁵⁶. A variant callset was created using the multiallelic calling and rare-variant calling model in BCFtools v1.8 with a prior probability of 0.99 after which a set filter excluded sites with a QUAL<20. Using this variant callset and the FastaAlternate ReferenceMaker function in GATK v3.7.0⁵⁷, individual FASTA files for each sample and gene were produced with homogeneous single nucleotide polymorphism sites replacing their respective reference bases while IUPAC ambiguity codes were used at heterozygous sites. FASTA files containing ambiguity codes were then trimmed to the coding region of each gene and manipulated to be merged by gene and individual using Linux command line utility commands. Pedigree-independent allelic phasing was performed on sequence data for each of the four genes of interest using DnaSP v5.10.1⁵⁸. Allelic phasing was later validated with available pedigree information from the KCCMR animal colony, and identified alleles were ultimately confirmed by amplifying them by PCR from cDNA libraries, cloning, and Sanger-sequencing individual products. Throughout this manuscript, alleles were defined only as those bearing at least one nonsynonymous single nucleotide polymorphism (SNP) compared to other observed alleles; synonymous SNPs were ignored.

Engineering HIV-1 Escape from Owl Monkey TRIMCyp

Crandell-Rees feline kidney (CRFK) cells (ATCC #CCL-94) stably expressing various Nancy Ma's owl monkey TRIMCyp alleles (Align. S3) were established via transduction using retroviral vectors to identify cyclophilin A-binding loops of various SIVs that escape owl monkey TRIMCyp restriction. To generate the retroviral vectors, human embryonic kidney (HEK) 293T cells (ATCC #CRL-3216) were seeded at a concentration of 1×10⁶ cells/well in 6-well dishes. Each well was transfected 24 h later with 2 μg of pLPCX plasmid from the LRCX Retroviral Vector Set (Takara #631511; empty or encoding the TRIMCyp allele of interest), 1 μg of pCS2-mGP plasmid containing murine leukemia virus (MLV) gag-pol⁵⁹, and 0.2 μg of pC-VSIV-G plasmid encoding the vesicular stomatitis Indiana virus (VSIV) glycoprotein (G) using the standard TransIT-293 protocol (Mirus Bio #MIR 2705). Supernatants were collected after 48 h, passed through a 0.2-μm filter, and used to transduce CRFK cells. After 24 h, medium containing 8 μg/mL of puromycin was added to select transduced cells. Cell lines were expanded and grown in puromycin for at least two weeks before expression of TRIMCyp constructs was detected by western blot.

Next, Applicants created different mutations to induce changes in the cyclophilin A-binding loop for screening of TRIMCyp escape (FIG. S4). The pMDLg/pRRE plasmid⁶⁰ containing HIV-1 gag-pol was used as a template for site-directed mutagenesis using PfuTurbo DNA polymerase (Agilent #600254)⁶¹. Viruses for single-cycle infection assays were packaged in 293T cells by co-transfection of plasmids encoding viral proteins and VSIV G, along with a transfer vector (pMDLg/pRRE, pRSV-Rev, pMD2.G, pRRLSIN.cPPT. PGK-GFP.WPRE; all available from Addgene). 293T cells were seeded into 6-well plates at a concentration of 1×10⁶ cells/well prior to transfection. After 48 h, supernatants containing viruses were harvested, filtered using a 0.45-micron filter, and frozen. The titer of viruses was determined on CRFK cells stably expressing human TRIM5 or owl monkey TRIMCyp by measuring percent green fluorescent protein (GFP)-positive cells along a volume gradient of virus supernatant. For infection assays, the modified CRFK cells were plated at a concentration of 7.5×10⁴ cells/well in 24-well plates and exposed to HIV-1 wildtype or variants containing mutations. Two days post-exposure, cells were fixed in 2% paraformaldehyde for 15 min, washed three times with 2 mL of FACS buffer (Dulbecco's Phosphate-Buffered Saline supplemented with 2% fetal bovine serum and 1 mM ethylenediamine tetraacetic acid (EDTA)), resuspended in 500 μl of FACS buffer, and analyzed by flow cytometry for expression of GFP. The percentage of each cell line that was GFP⁺ was calculated and normalized to the percentage of GFP⁺ cells in the control line (cells without TRIMCyp). All exposures were performed in triplicate using a single virus stock, and all results were confirmed using at least two experimental replicates.

Full-length HIV-1 engineered to bypass owl monkey TRIMCyp was then assessed for the ability to cause infection in two different immortalized cell lines, one from human and one from owl monkey. HIV-1 (Q23-17)⁶² was engineered to contain the four mutations re-constituting the SIVrem cyclophilin A-binding loop. This plasmid (wildtype or altered as described) was transfected into human cells to produce virus stocks, the titers of which were measured on TZM-bl cells (BEI Resources #ARP-8129), which are HeLa cells that express CD4, CCR5, CXCR4, and reporters that respond to HIV-1, specifically β-galactosidase (β-gal) or firefly luciferase, under the control of the HIV-1 long terminal repeat^{63,64}. Then, Hut78 (human) cells stably expressing human CCR5 were seeded into a 48-well dish at 10⁶ cells/well in a total of 600 μL of complete Roswell Park Memorial Institute (RPMI) medium. Three-striped owl monkey (Aotus trivirgatus) kidney OMK cells (ATCC #CRL-1556) stably expressing human CD4 and CCR5 were seeded into a 6-well dish at 200,000 cells/well in a total of 2 mL of complete Dulbecco's Modified Eagle Medium (DMEM). This is the only available immortalized owl monkey cell line. The next day, cells were exposed at an MOI=0.1 by spinoculation at 1,200×g for 90 min in the presence of 20 μg/mL diethylaminoethyl (DEAE)-dextran. After spinoculation, cells were washed five times with 1 mL of either RPMI (Hut78 cells) or Dulbecco's Phosphate-Buffered Saline (OMK cells). After washing, Hut78 cells were resuspended in 600 μL of complete RPMI in a 24-well dish, and OMK cells were provided 2 mL of complete Dulbecco's Modified Eagle Medium (DMEM). Supernatant aliquots were collected on 0, 3, 7, and 10 days post-exposure to quantify virus production. The ZeptoMetrix p24 Antigen ELISA kit (ZeptoMetrix #0801008) was used to quantify the amount of p24 (CA) in supernatant aliquots according to the manufacturer's instructions. A p24 (CA) protein standard provided with the kit was used to generate a standard curve.

Engineering HIV-1 Escape from Owl Monkey APOBEC3G

Through screening of 42 HIV-1, HIV-2, and SIV Vifs, Applicants identified SIVmne and SIVmac Vifs as capable of degrading Nancy Ma's owl monkey APOBEC3G (FIG. S7). Using an infection assay, Applicants then tested the effect of these Vifs on HIV-1 production in the presence of various APOBEC3Gs. Human APOBEC3G corresponds to GenBank #NM_021822. Nucleic acids encoding the four major owl monkey APOBEC3G alleles (FIG. S1, Align. S4) were PCR-amplified from owl monkey cDNA isolated from blood. 293T cells were plated in 24-well dishes at a density of 200,000 cells/well. Twenty-four hours later, cells were co-transfected with a plasmid encoding APOBEC3G (human at 20 ng and owl monkey at 200 ng) and 300 ng of a plasmid encoding HIV-1 (NL4-3) provirus or variants thereof: Δvif, SIVmne vif or SIVmac vif. Forty-eight hours post-transfection, virion-containing supernatants were collected and spun at 1,200×g for 5 min to remove cell debris. Equivalent volumes of supernatants were added to TZM-bl cells, which had been plated one day before in 96-well plates at 10,000 cells/well. Forty-eight hours post-exposure, cells were washed with Dulbecco's Phosphate-Buffered Saline and resuspended in Passive Lysis 5× Buffer (Promega #E194A) and incubated at room temperature for 15 min. Luciferase signal was activated using the Promega Luciferase Assay System (Promega #E1501) and detected using an Agilent BioTek Synergy HTX Multi-Mode Microplate Reader (Agilent #SILFA).

### Creation of HIV-1 Molecular Clones and Variants

Except where noted otherwise, viruses were constructed through manipulation of the pNL4-3 plasmid (BEI Resources #ARP-114), which encodes the full-length HIV-1 (NL4-3) provirus. Gibson cloning was performed to construct a vif-deleted version of pNL4-3, using the strategy shown in FIG. S24, and to replace the vif open reading frame with the vif from SIVmne (GenBank #U79412) or SIVmac (GenBank #M19499), using the cloning strategy shown in FIG. S25. To replace the cyclophilin A-binding loop with that of SIVrcm, Applicants used a gBlock of 440-bp length roughly centered in the region encoding the cyclophilin A-binding loop. The pNL4-3 proviruses with the inserted SIVmne or SIVmac vifs were used as templates for PCR amplification of Gibson fragments. These PCR products overlapped with the 5′ and 3′ ends of the SIVrem cyclophilin A-binding loop-encoding gBlock and the ampicillin resistance gene on the plasmid. The HIVnom2 plasmid was then used to construct a derivative clone containing the four mutations leading to the four amino acid substitutions that arose during in vivo adaptation in owl monkeys (HIVnom3). This clone was made through four consecutive site-directed mutagenesis reactions using primers designed using NEBaseChanger (https://nebasechanger.neb.com) and the standard protocol of the Q5 Site-Directed Mutagenesis Kit (New England Biolabs #E0554S).

### Hivnom Stock Production

Nancy Ma's owl monkeys were exposed to three viruses that were produced from molecular clones: HIVnom1, HIVnom2, or HIVnom3. Each virus was produced from the respective modified pNL4-3-derived plasmid. To create these stocks, 13×10⁶ 293T cells were plated in 15-cm tissue culture dishes in media without antibiotics. The following day, 20 μg of plasmid was transfected using the standard Trans-IT 293 transfection reagent protocol. Six hours post-transfection, media were removed and replaced with complete Dulbecco's Modified Eagle Medium (DMEM) containing 3% fetal bovine serum. Forty-eight hours later, media were removed and spun at 1,200×g for 5 min to remove cell debris. Virion-containing supernatants were clarified through 0.45-μm cellulose acetate syringe filters followed by concentration through 100-kDa Amicon columns (Millipore UFC910024). Concentrated virions were further purified through a 20% sucrose cushion at 21,000×g for 90 min at 4° C. Virus pellets were resuspended in 1 mL of Dulbecco's Phosphate-Buffered Saline and 100-mL aliquots were prepared for future use. To determine virus titers, one frozen aliquot was removed for testing. Virus titers were determined on TZM-bl cells using a β-galactosidase assay 65 using the protocol associated with NIH HIV Reagent Program entry #ARP-1470. To test replication of modified viruses, human SUP-T1 T-lymphoblasts (ATCC #CRL-1942) expressing both CD4 and CCR5 (and which naturally express CXCR4) were exposed at a MOI of 0.1 via spinoculation (500×g for 90 min at 30° C.). Cell supernatants were collected every other day for 12-14 days and frozen. Viruses in supernatants were quantified using both an SG-PERT assay as previously described 66 and via exposure of TZM-bl cells followed by measuring firefly luciferase luminescence.

### Nancy Ma's Owl Monkey Challenge Studies

In total, eleven adult Nancy Ma's owl monkeys (four females and seven males) ranging in age from 5-21 years of age were used in virus exposure studies. Animals were either pair-housed or single-housed in a designated study room outside of the main KCCMR colony but were otherwise maintained similarly to colony animals. Prior to beginning the study, animals were given complete physical examinations and had blood work performed to establish baseline data and ensure their health. Blood work included an in-house CBC analysis (Advia 120, Siemens Medical Solutions), serum chemistry analysis (Beckman Coulter #AU680), and flow cytometry analysis along with a baseline HIV-1 RNA quantification and serological screening assay, both performed by the University of Washington Retrovirology Laboratory.

Eight of the animals were infused with individual aliquots of stock virus that had been maintained in liquid nitrogen vapor following production in tissue culture. Two animals (CA and SO) were administered 1.27×10⁶ infectious units of HIVnom1, two animals (KI and RI) were administered 2.07×10⁶ infectious units of HIVnom2, and the remaining four animals (ME, SI, ST, and JI) were administered 9.3×10⁵ infectious units of HIVnom2 premixed with 9.1×10⁵ infectious units of HIVnom3 as part of the virus competition experiment.

The remaining three animals (AB, BO, and ON) were transfused with either citrate-phosphate-dextrose solution with adenine (CPDA)-1 preserved plasma, or a mixture of CPDA-1 preserved plasma and fresh CPDA-1 preserved whole blood obtained from previously exposed animals. Prior to the transfusion experiments, a major and minor crossmatch was performed by the KCCMR diagnostic laboratory on blood from the donor and recipient animals. No adverse reactions were identified between the donor and recipient animals in any of the crossmatch assays. Animal AB was transfused with a mixture of two frozen plasma aliquots from animal RI collected at week 21 and week 22 post-infection. The week-21 aliquot was 0.6 mL (viral RNA [vRNA]=2,760 genomes/mL) and the week-22 aliquot was 2.4 mL (vRNA=18,220 genomes/mL). Animals BO and ON were both transfused using a mixture of frozen plasma and fresh whole blood from animal AB. Animal BO received a 0.6-mL frozen plasma aliquot from week 4 post-infection (vRNA=895,304 genomes/mL) mixed with a 1.0-mL fresh whole blood sample from week 5 post-infection (vRNA=>21,000,000 genomes/mL). Animal ON received a 0.6-mL frozen plasma aliquot from week 7 post-infection (vRNA=11,104 genomes/mL) mixed with a 1.5-mL fresh whole blood sample from week 8 post-infection (vRNA=344 genomes/mL).

On the day of virus administration (day 0), each of the virus exposure animals was anesthetized with a combination of xylazine (Akorn 20 mg/mL, Gurnee, IL, USA) and ketamine (Dechra 100 mg/mL, Overland Park, KS, USA) at 2.3 mg/kg and 20 mg/kg total body weight, respectively. Aliquots of the virus stocks and the CPDA-1 preserved plasma were thawed on ice and maintained cold until administered. CPDA-1 preserved whole blood samples were administered within 1 h of collection. Viral solutions were slowly intravenously infused into one of the femoral veins of an anesthetized animal. Approximately 5 min after completion of the infusion, a blood sample was collected from the contralateral femoral vein into a K2 EDTA tube (BD Biosciences #367856 or #365974) for “day 0” vRNA quantification. Animals were monitored until fully recovered from anesthesia and then were observed a minimum of twice daily thereafter for the remainder of the study. Physical examinations and blood work analyses for health monitoring were performed on a periodic basis throughout the remainder of the study. The CBC data obtained for each study animal are provided in File S1.

On the final day of the study, animals were anesthetized using xylazine and ketamine and a blood sample was collected. The animals were then humanely euthanized using an overdose of pentobarbital and phenytoin (Euthasol, Virbac, Fort Worth, TX, USA), consistent with the regulatory guidelines of the American Veterinary Medical Association. Full diagnostic necropsies and tissue collections were performed for each animal.

Blood Sampling from Infected Animals

A maximum of 3 mL of blood was collected per week over the study from each of the eleven animals included in the virus exposure studies and deposited in: K2 EDTA tubes (BD Vacutainer, BD Biosciences #367856 or BD Microtainer, BD Biosciences #365974); serum clot tubes (BD Vacutainer, BD Biosciences #367812); or microcentrifuge tubes (Fisher Scientific #02-681-343) containing CPDA-1 anticoagulant (citric acid, sodium citrate, monobasic sodium phosphate, dextrose, and adenine) at a concentration of 0.14 mL of CPDA-1/mL of whole blood. The CPDA-1 anticoagulant was acquired aseptically from a commercial veterinary blood transfusion bag (IV Blood Transfusion bag, Jorgensen Laboratories #J0520X, Loveland, CO, USA). EDTA preserved blood was utilized for complete blood counts (CBC), to obtain plasma for vRNA and antibody assays and for the acquisition of peripheral blood mononuclear cells (PBMCs). Standard Ficoll (Ficoll-Paque Plus, GE Healthcare #17-1440-03) processing was used to obtain PBMCs. These cells were preserved in fetal bovine serum with 10% dimethyl sulfoxide (DMSO) at a density of 10⁶-10⁷ cells/mL and were maintained in liquid nitrogen vapor in cryogenic vials (Corning #431417) until needed. Sera derived from clot tubes were used for serum chemistry analyses. CPDA-1 preserved whole blood was used on the same day it was collected for blood transfusion experiments or the CPDA-1 plasma from these samples was collected and preserved in liquid nitrogen vapor in cryogenic vials (Corning #431417) until needed.

### Dexamethasone Treatment

Dexamethasone sodium phosphate (Hikma 10 mg/mL #00641036725, Eatontown, NJ, USA) was administered to immunosuppress some of the study animals. Treatment consisted of a once-daily subcutaneous injection. Initially, animals were administered dexamethasone at 1.5 mg/kg with the dose gradually increasing to 6 mg/kg over a 2-week period. Study animals were maintained at 6 mg/kg for an additional 1-2 weeks. Two of the study animals (SO and SI) were euthanized at the point when the dexamethasone dose was at its highest peak. The remaining study animals underwent a gradual withdrawal from dexamethasone over a 2-3-week period. Two study animals (ST and JI) were euthanized at the time point when dexamethasone dose was reduced to 1.5 mg/kg, just prior to full withdrawal from the drug. The remaining three animals (KI, RI, and BO) were maintained on study for several weeks following withdrawal of the dexamethasone. A 0.75-mL dose of oral potassium gluconate gel supplement (Renal K⁺, Vetoquinol, Ft Worth, TX) was administered daily to all animals on dexamethasone treatment to counteract the mild hypokalemia noted to occur in some animals.

### Determination of Viral Loads and Seroconversion

Plasma vRNA quantification was performed by the commercial human diagnostic services at the University of Washington Retrovirology Laboratory (UWRL; Department of Laboratory Medicine, Seattle, WA, USA) using the Abbott RealTime HIV-1 RNA assay (Abbot #06L18-090) in conjunction with RT-qPCR instrumentation, practices, and procedures standard to that laboratory. Briefly, 500 μL of frozen EDTA owl monkey plasma were submitted for analysis and then diluted to 1 mL by the laboratory prior to processing. The range of vRNA quantitation for the assay is 40-10,000,000 HIV-1 genomes/mL when using undiluted samples, although the range of vRNA reported in this study was 80-20,000,000 HIV-1 genomes per mL due to the dilution required for the animal samples. The laboratory reported four possible outcomes: 1) virus detected and a vRNA quantification in HIV-1 genomes/mL; 2) virus detected but numbers were below the lower limit of quantification (<80 HIV-1 genomes/mL); 3) virus detected but the numbers were above the upper limit of quantification (>20 million HIV-1 genomes/mL); 4) virus not detected. The vRNA assay was performed for each animal prior to the start of the study, on the day of exposure (day 0), and on multiple days throughout the study (File S1).

The initial HIV-1 seroconversion analyses in this study were also performed by UWRL through their two-step protocol for HIV-1/HIV-2 antibody detection. A 500-μL frozen plasma sample first underwent qualitative detection of HIV-1 p24 (CA) or HIV-1/HIV-2 antibodies in the plasma using the Architect HIV Ag/Ab Combo (Abbott Diagnostics #873766). Samples identified to be reactive to the first assay then underwent further analysis using the Bio-Rad Genius HIV1/2 system (Life Science). This second assay confirmed the presence of HIV-1 antibodies and also identified the specific type of antibodies (anti-p24 (CA), -p31(IN), -gp41, and -gp160) present in reactive samples. Given that only 3 mL of blood could be collected from each animal per week and that there were numerous blood-based assessments desired for each of the study animals, Applicants sought other means to document time-to-seroconversion that might require less than 500 μL of plasma. Through retrospective testing of plasma samples that had been previously analyzed by UWRL, one human point-of-care HIV assay (Determine HIV-1/2 Ag/Ab Combo test, Abbott Laboratories #7D2648) was identified to correlate well with the UWRL results. As this assay requires only 25 μL of plasma, it has been used to retrospectively determine the earliest date of seroconversion for study animals and also has been used to confirm the UWRL results. The results of all UWRL and point-of-care assays performed in this study are provided in File S1.

### Detection of Neutralizing Antibodies

Owl monkey plasma samples were obtained at different weeks post-exposure. Plasma samples were received frozen and heat-inactivated at 56° C. for 30 min. These samples were then mixed 1:5 in Dulbecco's Modified Eagle Medium (DMEM) containing 10% fetal bovine serum (FBS), L-glutamine, and penicillin/streptomycin, and 2-fold serially diluted. Dilutions were mixed 1:1 with HIVnom3 (4,000 infectious units/well) and incubated at 37° C. for 1 h. TZM-bl cell culture media (cells plated the day before at 1.1×10⁴ cells/well in a 96-well plate) were removed and replaced with virus/plasma mixtures. TZM-bl cells contain an integrated Tat-inducible firefly luciferase gene. The samples were incubated for 48 h, after which the cells were lysed using the Luciferase Assay System (Promega #E1500) and relative light units (RLUs) were measured using a luminometer. Uninfected cells were used to correct for background firefly luciferase activity. Samples were normalized to cells exposed to virus but not exposed to plasma. Infectivity surpassing 100% in some samples suggests incomplete heat inactivation of virus in samples with very high viral loads.

### Sequencing of HIVnom Proviruses

Virus evolution was monitored throughout animal studies. PBMC samples were obtained from owl monkeys at multiple timepoints of infection, and genomic DNA was extracted using the DNeasy Blood & Tissue Kit (QIAGEN #69504). Twelve primer pairs were designed for nested PCR to amplify six separate overlapping amplicons (900-2,500 bp each) from integrated pro-viral DNA representing the entire HIVnom genome. PCR was performed using the Q5 High-Fidelity 2× Master Mix (NEB #M0492S). The first of each nested set of amplicons were gel-extracted using the Wizard SV Gel and PCR Clean-Up System (Promega #A9281) and the resulting products were diluted 1:100 in nuclease-free water before being used as templates for a second round of PCR. Both strands of the second amplicons were Sanger-sequenced (Quintara Biosciences). Single nucleotide polymorphisms (SNPs) that had reached fixation in the proviral pool were flagged at each time point in each animal.

For dually-exposed (HIVnom2+HIVnom3) animals, Sanger sequencing was used to determine the presence of each virus over time. Genomic DNA was prepared from PBMCs at various timepoints after exposure, and then used as template for nested PCR and Sanger-sequencing of proviral DNA as described above. Samples were selectively sequenced at the four sites that differ between the original and adapted virus. The complete virus genomes were sequenced from gDNA isolated at time of necropsy to identify any new mutations that may have arisen during infection.

### Histopathological and In Situ Hybridization Tissue Analyses

Tissues collected at necropsy were immediately placed in 10% neutral buffered formalin and then processed into paraffin blocks within 72 h of collection. For each animal, 5-μm sections were prepared from a minimum of 44 unique tissues. These sections were stained using hematoxylin and eosin (H&E) by the KCCMR Histology Laboratory using instruments, practices, and procedures standard to that laboratory. The resultant slides were reviewed by a boarded veterinary pathologist (G.K.W.) for pathologic lesions, and specifically for any changes typical of lentiviral infections or immune dysfunction.

Additional (axial, inguinal, mandibular, and mesenteric) lymph node and pancreas sections were obtained from HIVnom3-infected animals (AB, ON, ME, SI, ST, and JI) for analysis through RNA in situ hybridization. Cell culture preparations were initially used for optimization of the RNA in situ hybridization assay. In brief, TZMbl cells stably expressing human CD4, CCR5, and CXCR4 were exposed to varying volumes of concentrated HIVnom3 virus stock (0.1, 1, or 10 μL) or exposed to media alone. Following 48 h of incubation, cells were collected, fixed (10% Neutral Buffered Formalin, MilliporeSigma #HT501128-4L), and permeabilized using ethanol. Cells were then suspended in agarose gel (Fisher Scientific, HistoGel #22-110-678) and processed into paraffin blocks for sectioning. Negative and positive control tissues samples were also used for optimization of the RNA in situ hybridization assay and, additionally, for comparison to the infected owl monkey lymph nodes. As pancreas is not generally considered a primary reservoir tissue in chronic HIV-1 infection, Applicants expected HIV-1 pancreatic staining to be negligible in infected animals and processed this tissue as an internal negative control. Lymph node sections were obtained from uninfected (naïve) animals and served as an external negative control. Applicants obtained paraffin-embedded lymph node blocks derived from human patients chronically infected with HIV-1 to serve as positive control tissues. The human tissues were obtained through a commercial biobank (Aurora Diagnostics, Palm Beach FL, USA [Formerly Aurora Research Institute, LLC, Las Vegas, NV, USA]) under approved IRB protocols. All human tissues had undergone de-identification of personal health information (PHI) in accordance with the HIPAA Privacy Rule prior to being provided to KCCMR. Serial 5-μm sections were collected from the various cell and tissue blocks for use in the RNA in situ hybridization assays.

RNA in situ hybridization processing of the experimental and control tissues was performed by the Pathology Services Core at the University of North Carolina at Chapel Hill using instruments, practices, and procedures standard to that laboratory. In brief, RNA in situ hybridization processing used a fully automated system (Leica BOND RX, Leica biosystems) and a commercially available detection reagent kit (ACD Bio #322100). One RNA probe set (ACD Bio #313908) was applied to one of the serial sections to assess general RNA viability through analysis of the transcript from the human housekeeping gene, peptidylprolyl isomerase B (PPIB). A second RNA probe set (ACD Bio #312038), recognizing the transcript from the Bacillus subtilis dihydrodipicolinate reductase (dapB) gene, was applied to another serial section as a negative control to identify background/nonspecific staining. A third RNA probe set (ACD Bio #416118), comprised of 78 probe pairs spanning the HIV-1 NL4-3 genome, was applied to additional serial sections for the detection of HIV-1 RNA. The specific tissues undergoing RNA in situ hybridization analysis and the results of these analyses are provided in FIGS. S22 and S23.

### Supplemental Methods for Supplementary FIG. S6

For the creation of a Vif library, 40 unique vif sequences (File S2) from eight HIV-1 isolates, two HIV-2 isolates, and 30 different SIVs were codon-optimized using Codon Optimization OnLine (COOL) 70. All vifs were then synthesized as gBlocks (Integrated DNA Technologies) and cloned into the retroviral expression vector pLPCX with a C-term HA-tag. The human APOBEC3G sequence used matches GenBank #NM_021822.

Degradation assays were used to test each Vif in the library for the ability to degrade owl monkey APOBEC3G (the variant encoded by each major allele in the colony was tested). Human APOBEC3G served as a positive control. 293T cells were plated into 24-well dishes at a concentration of 200,000 cells/well in Dulbecco's Modified Eagle Medium (DMEM) without antibiotics. Twenty-four hours later, they were co-transfected with two plasmids: one expressing APOBEC3G (human 25 ng or owl monkey 200 ng to produce equivalent expression) and one expressing Vif (200 ng) using TransIT-293 transfection reagent (Mirus Bio cat #MIR 2705). Forty-eight hours post-transfection, cells were washed in Dulbecco's Phosphate-Buffered Saline (PBS) and lysed in NP-40 cell lysis buffer (50 mM Tris-HCl pH 8.0, 150 mM NaCl, 1% Nonidet P-40, pH 7.8) supplemented with IX Complete protease inhibitor (Sigma cat #11873580001). Cells were rotated in lysis buffer at 4° C. for 45 min and whole cell extracts were cleared by spinning at 16,000×g for 15 minutes at 4° C. Protein concentrations were determined using the Pierce BCA protein assay (Thermo Scientific cat #23227). Equal amounts of protein (10 μg) were resolved using 12% TGX stain-free FastCast acrylamide gels (Biorad cat #1610185) and transferred onto Immobilon-P PVDF membrane (EMD Millipore cat #IPVH07850). Blots were blocked for 30 min at room temperature in 3% milk. HA-tagged APOBEC3G and Vif proteins were detected using a 1:5,000 dilution of a mouse anti-HA antibody conjugated with horseradish peroxidase (Thermo Scientific cat #MA1-91878-HRP). As a loading control endogenous actin beta was detected using a 1:5,000 dilution of a mouse anti-actin beta antibody (Cell Signaling Technology cat #3700S). A 1:10,000 dilution of a goat anti-mouse horseradish peroxidase-conjugated antibody (Promega cat #W4021) was used as a secondary probe. Chemiluminescence was activated with the ECL prime western blotting detection reagent (GE Healthcare cat #RPN2236) and imaged on a BioRad ChemiDoc imaging system.

### Biosafety Considerations

This study utilized viruses that are presumably infectious to people, because they replicate on a human T cell line (FIG. S14). All procedures instituted for risk minimization and management of human virus exposures during in vitro work were overseen and approved by Environmental Health and Safety and were approved by the Institutional Biosafety Committee (IBC Approval #BA18-MCDB-SAW-01) at the University of Colorado Boulder, Boulder, Colorado, USA. Virus stocks and biologic samples from infected animals were handled with biosafety level 3 operating procedures within a biosafety level 2 physical facility. Prior to animal testing, all virus stocks were first confirmed by a third-party laboratory (VELA Diagnostics) to be susceptible to the drugs used in standard post-exposure prophylaxis treatment, typically tenofovir, emtricitabine, and raltegravir³³. This ensured that any research personnel, in case of accidental exposure to viruses or virus-exposed animals, could be successfully treated with standard post-exposure protocols if necessary. All procedures instituted for risk minimization and management of human virus exposures during in vivo experimentation were overseen by Environmental Health and Safety and were approved by the Institutional Biosafety Committee (IBC Approval #s: RM00002884-RN00 and RM00005639-RN00) at The University of Texas, MD Anderson Cancer Center.

TABLE 2
Antibodies used for flow cytometry of owl monkey blood
Compensation controls

| Antibody | Cat# | Vendor | Status |
|---|---|---|---|
| PerCP Mouse Anti-Human CD3 (SP34-2) | 552851 | BD | 5 and 7 color panel |
| PE Mouse Anti-Human CD3 (SP34-2) | 552127 | BD | 5 and 7 color panel |
| FITC Mouse Anti-Human CD3 (SP34) | 556611 | BD | 5 and 7 color panel |
| APC Mouse Anti-Human CD3 (SP34-2) | 557597 | BD | 5 and 7 color panel |
| BV650 Mouse Anti-Human CD3 (SP34-2) | 563916 | BD | 5 and 7 color panel |
| AF700 Mouse Anti-Human CD3 (SP34-2) | 561805 | BD | 7 color panel |
| BV786 Mouse Anti-Human CD3 (SP34-2) | 563918 | BD | 7 color panel |

Isotype Controls

| Antibody | Cat# | Vendor | Status |
|---|---|---|---|
| PerCP Mouse IgG1 isotype control (MOPC-21) | 559425 | BD | 5 and 7 color panel |
| FITC Mouse IgG2a isotype control | MG2A01 | LifeTech | 5 and 7 color panel |
| BV650 Mouse IgG1 isotype control (X40) | 563231 | BD | 5 and 7 color panel |
| APC Mouse IgG1 isotype control | 340442 | BD | 5 and 7 color panel |
| PE Mouse IgG3 isotype control | 556659 | BD | 5 and 7 color panel |
| AF700 IgG2a isotype control (G155-178) | 557880 | BD | 7 color panel |
| BV786 IgG1 isotype control (X40) | 563330 | BD | 7 color panel |

Samples

| Antibody | Cat# | Vendor | Status |
|---|---|---|---|
| CD20 APC (L27) | 340941 | BD | 5 and 7 color panel |
| PE Mouse Anti-Human CD195 CCR5 (3A9) | 556042 | BD | 5 and 7 color panel |
| PerCP Mouse Anti-Human CD4 (L200) | 550631 | BD | 5 and 7 color panel |
| CD8 FITC Human (3B5) | MHCD0801 | Invitrogen | 5 and 7 color panel |
| BV650 Mouse Anti-Human CD3 (SP34-2) | 563916 | BD | 5 color panel only |
| BV650 Mouse Anti-Human CD16 (3G8) | 563892 | BD | 7 color panel only |
| AF700 Mouse Anti-Human CD14 (M5E2) | 557923 | BD | 7 color panel only |
| BV786 Mouse Anti-Human CD3 (SP34-2) | 563918 | BD | 7 color panel only |

### REFERENCES

1. Fauci A S. 2017. An HIV Vaccine is Essential for Ending the HIV/AIDS Pandemic. JAMA 318:1535.
2. Meyerson N R, Sharma A, Wilkerson G K, Overbaugh J, Sawyer S L. 2015. Identification of owl monkey CD4 receptors broadly compatible with early-stage HIV-1 Isolates. J Virol 89:8611-8622.
3. Warren C J, Meyerson N R, Dirasantha O, Feldman E R, Wilkerson G K, Sawyer S L. 2019. Selective use of primate CD4 receptors by HIV-1. PLOS Biol 17: e3000304.
4. Humes D, Emery S, Laws E, Overbaugh J. 2012. A Species-Specific Amino Acid Difference in the Macaque CD4 Receptor Restricts Replication by Global Circulating HIV-1 Variants Representing Viruses from Recent Infection. J Virol 86:12472-12483.
5. Nisole S, Lynch C, Stoye J P, Yap M W. 2004. A Trim5-cyclophilin A fusion protein found in owl monkey kidney cells can restrict HIV-1. Proc National Acad Sci 101:13324-13328.
6. Sayah D M, Sokolskaja E, Berthoux L, Luban J. 2004. Cyclophilin A retrotransposition into TRIM5 explains owl monkey resistance to HIV-1. Nature 430:569-573.
7. Sauter D, Kirchhoff F. 2019. Key viral adaptations preceding the AIDS pandemic. Cell Host Microbe 25:27-38.
8. Wong S K, Connole M, Sullivan J S, Choc H, Carville A, Farzan M. 2009. A New World Primate Deficient in Tetherin-Mediated Restriction of Human Immunodeficiency Virus Type 1. J Virol 83:8771-8780.
9. Adachi A, Gendelman H E, Koenig S, Folks T, Willey R, Rabson A, Martin M A. 1986. Production of acquired immunodeficiency syndrome-associated retrovirus in human and nonhuman cells transfected with an infectious molecular clone. J Virol 59:284-291.
10. Okoye A A, et al . . . 2018. Early antiretroviral therapy limits SIV reservoir establishment to delay or prevent post-treatment viral rebound. Nat Med 24:1430-1440.
11. Robb M L, Eller L A, Kibuuka H, Rono K, Maganga L, Nitayaphan S, Kroon E, SaFK, Sinei S, Sriplienchan S, Jagodzinski L L, Malia J, Manak M, Souza M S de, Tovanabutra S, Sanders-Buell E, Rolland M, Dorsey-Spitz J, Eller M A, Milazzo M, Li Q, Lewandowski A, Wu H, Swann E, O'Connell R J, Peel S, Dawson P, Kim J H, Michael N L, Team R 217 S. 2016. Prospective Study of Acute HIV-1 Infection in Adults in East Africa and Thailand. New Engl J Medicine 374:2120-2130.
12. Barden A, Phillips M, Hill L M, Fletcher E M, Mas E, Loh P S, French M A, Ho K M, Mori T A, Corcoran T B. 2018. Antiemetic doses of dexamethasone and their effects on immune cell populations and plasma mediators of inflammation resolution in healthy volunteers. Prostaglandins, Leukot Essent Fat Acids 139:31-39.
13. Colby D J. et al., group T R study. 2018. Rapid HIV RNA rebound after antiretroviral treatment interruption in persons durably suppressed in Fiebig I acute HIV infection. Nat Med 24:923-926.
14. Henrich T J, et al., 2017. HIV-1 persistence following extremely early initiation of antiretroviral therapy (ART) during acute HIV-1 infection: An observational study. PLOS Med 14: c1002417.
15. Hatziioannou T, Prete G Q D, Keele B F, Estes J D, McNatt M W, Bitzegeio J, Raymond A, Rodriguez A, Schmidt F, Trubey C M, Smedley J, Piatak M, KewalRamani V N, Lifson J D, Bieniasz P D. 2014. HIV-1-induced AIDS in monkeys. Science 344:1401-1405.
16. Ambrose Z, KewalRamani V N, Bieniasz P D, Hatziioannou T. 2007. HIV/AIDS: in search of an animal model. Trends Biotechnol 25:333-337.
17. Li H, et al., 2016. Envelope residue 375 substitutions in simian-human immunodeficiency viruses enhance CD4 binding and replication in rhesus macaques. Proc National Acad Sci 113: E3413-E3422.
18. Joseph S B, Swanstrom R. 2018. The evolution of HIV-1 entry phenotypes as a guide to changing target cells. J Leukocyte Biol 103:421-431.
19. Pugach P, Kuhmann S E, Taylor J, Marozsan A J, Snyder A, Ketas T, Wolinsky S M, Korber B T, Moore J P. 2004. The prolonged culture of human immunodeficiency virus type 1 in primary lymphocytes increases its sensitivity to neutralization by soluble CD4. Virology 321:8-22.
20. Wrin T, Loh T P, Vennari J C, Schuitemaker H, Nunberg J H. 1995. Adaptation to persistent growth in the H9 cell line renders a primary isolate of human immunodeficiency virus type 1 sensitive to neutralization by vaccine sera. J Virol 69:39-48.
21. Munro J B, Gorman J, Ma X, Zhou Z, Arthos J, Burton D R, Koff W C, Courter J R, Smith A B, Kwong P D, Blanchard S C, Mothes W. 2014. Conformational dynamics of single HIV-1 envelope trimers on the surface of native virions. Science 346:759-763.
22. Unutmaz D, KewalRamani V N, Littman D R. 1998. G protein-coupled receptors in HIV and SIV entry: New perspectives on lentivirus-host interactions and on the utility of animal models. Semin Immunol 10:225-236.
23. Sina S T, Ren W, Cheng-Mayer C. 2011. Coreceptor use in nonhuman primate models of HIV infection. J Transl Med 9: S7.
24. Riddick N E, Hermann E A, Loftin L M, Elliott S T, Wey W C, Cervasi B, Taaffe J, Engram J C, Li B, Else J G, Li Y, Hahn B H, Derdeyn C A, Sodora D L, Apetrei C, Paiardini M, Silvestri G, Collman R G. 2010. A novel CCR5 mutation common in sooty mangabeys reveals SIVsmm infection of CCR5-null natural hosts and efficient alternative coreceptor use in vivo. Plos Pathog 6: e1001064.
25. Wetzel K S, Yi Y, Elliott S T C, Romero D, Jacquelin B, Hahn B H, Muller-Trutwin M, Apetrei C, Pandrea I, Collman R G. 2017. CXCR6-Mediated Simian Immunodeficiency Virus SIVagmSab Entry into Sabacus African Green Monkey Lymphocytes Implicates Widespread Use of Non-CCR5 Pathways in Natural Host Infections. J Virol 91.
26. Schmidt F, Keele B F, Prete G Q D, Voronin D, Fennessey C M, Soll S, Kane M, Raymond A, Gifford R J, KewalRamani V, Lifson J D, Bieniasz P D, Hatziioannou T. 2019. Derivation of simian tropic HIV-1 infectious clone reveals virus adaptation to a new host. Proc National Acad Sci 116:10504-10509.
27. Bolger A M, Lohse M, Usadel B. 2014. Trimmomatic: a flexible trimmer for Illumina sequence data. Bioinformatics 30:2114-2120.
28. Andrews S. FastQC. http://www.bioinformatics.babraham.ac.uk/projects/fastqc.
29. Li H, Durbin R. 2009. Fast and accurate short read alignment with Burrows-Wheeler transform. Bioinformatics 25:1754-1760.
30. Li H, Handsaker B, Wysoker A, Fennell T, Ruan J, Homer N, Marth G, Abecasis G, Durbin R, Subgroup 1000 Genome Project Data Processing. 2009. The Sequence Alignment/Map format and SAMtools. Bioinformatics 25:2078-2079.
31. Danecek P, Bonfield J K, Liddle J, Marshall J, Ohan V, Pollard M O, Whitwham A, Keane T, McCarthy S A, Davies R M, Li H. 2021. Twelve years of SAMtools and BCFtools. GigaScience 10: giab008.
32. Auwera G V der, O'Connor B. 2020. Genomics in the Cloud: Using Docker, GATK, and WDL in Terra.
33. Librado P, Rozas J. 2009. DnaSP v5: a software for comprehensive analysis of DNA polymorphism data. Bioinformatics 25:1451-1452.
34. Yamashita M, Emerman M. 2004. Capsid Is a dominant determinant of retrovirus infectivity in nondividing cells. J Virol 78:5670-5678.
35. Dull T, Zufferey R, Kelly M, Mandel R J, Nguyen M, Trono D, Naldini L. 1998. A third-generation lentivirus vector with a conditional packaging system. Journal of Virology 72:8463-8471.
36. Meyerson N R, Warren C J, Vieira DASA, Diaz-Griffero F, Sawyer S L. 2018. Species-specific vulnerability of RanBP2 shaped the evolution of SIV as it transmitted in African apes. PloS Pathog 14: e1006906.
37. Kimpton J, Emerman M. 1992. Detection of replication-competent and pseudotyped human immunodeficiency virus with a sensitive cell line on the basis of activation of an integrated beta-galactosidase gene. Journal of Virology 66:2232-2239.
38. Vermeire J, Naessens E, Vanderstraeten H, Landi A, Iannucci V, Nuffel A V, Taghon T, Pizzato M, Verhasselt B. 2012. Quantification of Reverse Transcriptase Activity by Real-Time PCR as a Fast and Accurate Method for Titration of HIV, Lenti- and Retroviral Vectors. PLOS ONE 7: e50859.
39. Leigh J W, Bryant D. 2015. popart: full-feature software for haplotype network construction. Methods Ecol Evol 6:1110-1116.
40. Zhen A, Wang T, Zhao K, Xiong Y, Yu X-F. 2010. A Single Amino Acid Difference in Human APOBEC3H Variants Determines HIV-1 Vif Sensitivity. J Virol 84:1902-1911.
41. Poss M, Overbaugh J. 1999. Variants from the diverse virus population identified at seroconversion of a clade A human immunodeficiency virus type 1-infected woman have distinct biological properties. Journal of Virology 73:5255-5264.
42. Ochsenbauer C, Edmonds T G, Ding H, Keele B F, Decker J, Salazar M G, Salazar-Gonzalez J F, Shattock R, Haynes B F, Shaw G M, Hahn B H, Kappes J C. 2012. Generation of Transmitted/Founder HIV-1 Infectious Molecular Clones and Characterization of Their Replication Capacity in CD4 T Lymphocytes and Monocyte-Derived Macrophages. J Virol 86:2715-2728.
43. Xu H, Svarovskaia E S, Barr R, Zhang Y, Khan M A, Strebel K, Pathak V K. 2004. A single amino acid substitution in human APOBEC3G antiretroviral enzyme confers resistance to HIV-1 virion infectivity factor-induced depletion. Proc National Acad Sci 101:5652-5657.
44. Chin J X, Chung B K-S, Lee D-Y. 2014. Codon Optimization OnLine (COOL): a web-based multi-objective optimization platform for synthetic gene design. Bioinformatics 30:2210-2212.
45. Kukkonen S, Martinez-Viedma M D P, Kim N, Manrique M, Aldovini A. 2014. HIV-1 Tat second exon limits the extent of Tat-mediated modulation of interferon-stimulated genes in antigen presenting cells. Retrovirology 11:30.
46. Wei X, Decker J M, Liu H, Zhang Z, Arani R B, Kilby J M, Saag M S, Wu X, Shaw G M, Kappes J C. 2002. Emergence of Resistant Human Immunodeficiency Virus Type 1 in Patients Receiving Fusion Inhibitor (T-20) Monotherapy. ANTIMICROBIAL AGENTS AND CHEMOTHERAPY.
47. Gamble T R, Vajdos F F, Yoo S, Worthylake D K, Houseweart M, Sundquist W I, Hill C P. 1996. Crystal structure of human cyclophilin A bound to the amino-terminal domain of HIV-1 capsid. Cell 87:1285-1294.
48. Bichel K, Price A J, Schaller T, Towers G J, Freund S M, James L C. 2013. HIV-1 capsid undergoes coupled binding and isomerization by the nuclear pore protein NUP358. Retrovirology 10:81.
49. Ylinen L M J, Price A J, Rasaiyaah J, Hue S phane, Rose N J, FlaviaMarzetta, James L C, Towers G J. 2010. Conformational Adaptation of Asian Macaque TRIMCyp Directs Lineage Specific Antiviral Activity. PLOS Pathogens 6: e1001062.

SEQUENCE LISTING/LEGEND

SEQ ID NO. 1
DNA
NL4-3 strain
HIV-1
TGGAAGGGCTAATTTGGTCCCAAAAAAGACAAGAGATCCTTGATCTGTGGATCTACCACACACAAGGCTA
CTTCCCTGATTGGCAGAACTACACACCAGGGCCAGGGATCAGATATCCACTGACCTTTGGATGGTGCTTC
AAGTTAGTACCAGTTGAACCAGAGCAAGTAGAAGAGGCCAATGAAGGAGAGAACAACAGCTTGTTACACC
CTATGAGCCAGCATGGGATGGAGGACCCGGAGGGAGAAGTATTAGTGTGGAAGTTTGACAGCCTCCTAGC
ATTTCGTCACATGGCCCGAGAGCTGCATCCGGAGTACTACAAAGACTGCTGACATCGAGCTTTCTACAAG
GGACTTTCCGCTGGGGACTTTCCAGGGAGGTGTGGCCTGGGCGGGACTGGGGAGTGGCGAGCCCTCAGAT
GCTACATATAAGCAGCTGCTTTTTGCCTGTACTGGGTCTCTCTGGTTAGACCAGATCTGAGCCTGGGAGC
TCTCTGGCTAACTAGGGAACCCACTGCTTAAGCCTCAATAAAGCTTGCCTTGAGTGCTCAAAGTAGTGTG
TGCCCGTCTGTTGTGTGACTCTGGTAACTAGAGATCCCTCAGACCCTTTTAGTCAGTGTGGAAAATCTCT
AGCAGTGGCGCCCGAACAGGGACTTGAAAGCGAAAGTAAAGCCAGAGGAGATCTCTCGACGCAGGACTCG
GCTTGCTGAAGCGCGCACGGCAAGAGGCGAGGGGCGGCGACTGGTGAGTACGCCAAAAATTTTGACTAGC
GGAGGCTAGAAGGAGAGAGATGGGTGCGAGAGCGTCGGTATTAAGCGGGGGAGAATTAGATAAATGGGAA
AAAATTCGGTTAAGGCCAGGGGGAAAGAAACAATATAAACTAAAACATATAGTATGGGCAAGCAGGGAGC
TAGAACGATTCGCAGTTAATCCTGGCCTTTTAGAGACATCAGAAGGCTGTAGACAAATACTGGGACAGCT
ACAACCATCCCTTCAGACAGGATCAGAAGAACTTAGATCATTATATAATACAATAGCAGTCCTCTATTGT
GTGCATCAAAGGATAGATGTAAAAGACACCAAGGAAGCCTTAGATAAGATAGAGGAAGAGCAAAACAAAA
GTAAGAAAAAGGCACAGCAAGCAGCAGCTGACACAGGAAACAACAGCCAGGTCAGCCAAAATTACCCTAT
AGTGCAGAACCTCCAGGGGCAAATGGTACATCAGGCCATATCACCTAGAACTTTAAATGCATGGGTAAAA
GTAGTAGAAGAGAAGGCTTTCAGCCCAGAAGTAATACCCATGTTTTCAGCATTATCAGAAGGAGCCACCC
CACAAGATTTAAATACCATGCTAAACACAGTGGGGGGACATCAAGCAGCCATGCAAATGTTAAAAGAGAC
CATCAATGAGGAAGCTGCAGAATGGGATAGATTGCATCCAGTGCATGCAGGGCCTATTGCACCAGGCCAG
ATGAGAGAACCAAGGGGAAGTGACATAGCAGGAACTACTAGTACCCTTCAGGAACAAATAGGATGGATGA
CACATAATCCACCTATCCCAGTAGGAGAAATCTATAAAAGATGGATAATCCTGGGATTAAATAAAATAGT
AAGAATGTATAGCCCTACCAGCATTCTGGACATAAGACAAGGACCAAAGGAACCCTTTAGAGACTATGTA
GACCGATTCTATAAAACTCTAAGAGCCGAGCAAGCTTCACAAGAGGTAAAAAATTGGATGACAGAAACCT
TGTTGGTCCAAAATGCGAACCCAGATTGTAAGACTATTTTAAAAGCATTGGGACCAGGAGCGACACTAGA
AGAAATGATGACAGCATGTCAGGGAGTGGGGGGACCCGGCCATAAAGCAAGAGTTTTGGCTGAAGCAATG
AGCCAAGTAACAAATCCAGCTACCATAATGATACAGAAAGGCAATTTTAGGAACCAAAGAAAGACTGTTA
AGTGTTTCAATTGTGGCAAAGAAGGGCACATAGCCAAAAATTGCAGGGCCCCTAGGAAAAAGGGCTGTTG
GAAATGTGGAAAGGAAGGACACCAAATGAAAGATTGTACTGAGAGACAGGCTAATTTTTTAGGGAAGATC
TGGCCTTCCCACAAGGGAAGGCCAGGGAATTTTCTTCAGAGCAGACCAGAGCCAACAGCCCCACCAGAAG
AGAGCTTCAGGTTTGGGGAAGAGACAACAACTCCCTCTCAGAAGCAGGAGCCGATAGACAAGGAACTGTA
TCCTTTAGCTTCCCTCAGATCACTCTTTGGCAGCGACCCCTCGTCACAATAAAGATAGGGGGGCAATTAA
AGGAAGCTCTATTAGATACAGGAGCAGATGATACAGTATTAGAAGAAATGAATTTGCCAGGAAGATGGAA
ACCAAAAATGATAGGGGGAATTGGAGGTTTTATCAAAGTAAGACAGTATGATCAGATACTCATAGAAATC
TGCGGACATAAAGCTATAGGTACAGTATTAGTAGGACCTACACCTGTCAACATAATTGGAAGAAATCTGT
TGACTCAGATTGGCTGCACTTTAAATTTTCCCATTAGTCCTATTGAGACTGTACCAGTAAAATTAAAGCC
AGGAATGGATGGCCCAAAAGTTAAACAATGGCCATTGACAGAAGAAAAAATAAAAGCATTAGTAGAAATT
TGTACAGAAATGGAAAAGGAAGGAAAAATTTCAAAAATTGGGCCTGAAAATCCATACAATACTCCAGTAT
TTGCCATAAAGAAAAAAGACAGTACTAAATGGAGAAAATTAGTAGATTTCAGAGAACTTAATAAGAGAAC
TCAAGATTTCTGGGAAGTTCAATTAGGAATACCACATCCTGCAGGGTTAAAACAGAAAAAATCAGTAACA
GTACTGGATGTGGGCGATGCATATTTTTCAGTTCCCTTAGATAAAGACTTCAGGAAGTATACTGCATTTA
CCATACCTAGTATAAACAATGAGACACCAGGGATTAGATATCAGTACAATGTGCTTCCACAGGGATGGAA
AGGATCACCAGCAATATTCCAGTGTAGCATGACAAAAATCTTAGAGCCTTTTAGAAAACAAAATCCAGAC
ATAGTCATCTATCAATACATGGATGATTTGTATGTAGGATCTGACTTAGAAATAGGGCAGCATAGAACAA
AAATAGAGGAACTGAGACAACATCTGTTGAGGTGGGGATTTACCACACCAGACAAAAAACATCAGAAAGA
ACCTCCATTCCTTTGGATGGGTTATGAACTCCATCCTGATAAATGGACAGTACAGCCTATAGTGCTGCCA
GAAAAGGACAGCTGGACTGTCAATGACATACAGAAATTAGTGGGAAAATTGAATTGGGCAAGTCAGATTT
ATGCAGGGATTAAAGTAAGGCAATTATGTAAACTTCTTAGGGGAACCAAAGCACTAACAGAAGTAGTACC
ACTAACAGAAGAAGCAGAGCTAGAACTGGCAGAAAACAGGGAGATTCTAAAAGAACCGGTACATGGAGTG
TATTATGACCCATCAAAAGACTTAATAGCAGAAATACAGAAGCAGGGGCAAGGCCAATGGACATATCAAA
TTTATCAAGAGCCATTTAAAAATCTGAAAACAGGAAAGTATGCAAGAATGAAGGGTGCCCACACTAATGA
TGTGAAACAATTAACAGAGGCAGTACAAAAAATAGCCACAGAAAGCATAGTAATATGGGGAAAGACTCCT
AAATTTAAATTACCCATACAAAAGGAAACATGGGAAGCATGGTGGACAGAGTATTGGCAAGCCACCTGGA
TTCCTGAGTGGGAGTTTGTCAATACCCCTCCCTTAGTGAAGTTATGGTACCAGTTAGAGAAAGAACCCAT
AATAGGAGCAGAAACTTTCTATGTAGATGGGGCAGCCAATAGGGAAACTAAATTAGGAAAAGCAGGATAT
GTAACTGACAGAGGAAGACAAAAAGTTGTCCCCCTAACGGACACAACAAATCAGAAGACTGAGTTACAAG
CAATTCATCTAGCTTTGCAGGATTCGGGATTAGAAGTAAACATAGTGACAGACTCACAATATGCATTGGG
AATCATTCAAGCACAACCAGATAAGAGTGAATCAGAGTTAGTCAGTCAAATAATAGAGCAGTTAATAAAA
AAGGAAAAAGTCTACCTGGCATGGGTACCAGCACACAAAGGAATTGGAGGAAATGAACAAGTAGATAAAT
TGGTCAGTGCTGGAATCAGGAAAGTACTATTTTTAGATGGAATAGATAAGGCCCAAGAAGAACATGAGAA
ATATCACAGTAATTGGAGAGCAATGGCTAGTGATTTTAACCTACCACCTGTAGTAGCAAAAGAAATAGTA
GCCAGCTGTGATAAATGTCAGCTAAAAGGGGAAGCCATGCATGGACAAGTAGACTGTAGCCCAGGAATAT
GGCAGCTAGATTGTACACATTTAGAAGGAAAAGTTATCTTGGTAGCAGTTCATGTAGCCAGTGGATATAT
AGAAGCAGAAGTAATTCCAGCAGAGACAGGGCAAGAAACAGCATACTTCCTCTTAAAATTAGCAGGAAGA
TGGCCAGTAAAAACAGTACATACAGACAATGGCAGCAATTTCACCAGTACTACAGTTAAGGCCGCCTGTT
GGTGGGCGGGGATCAAGCAGGAATTTGGCATTCCCTACAATCCCCAAAGTCAAGGAGTAATAGAATCTAT
GAATAAAGAATTAAAGAAAATTATAGGACAGGTAAGAGATCAGGCTGAACATCTTAAGACAGCAGTACAA
ATGGCAGTATTCATCCACAATTTTAAAAGAAAAGGGGGGATTGGGGGGTACAGTGCAGGGGAAAGAATAG
TAGACATAATAGCAACAGACATACAAACTAAAGAATTACAAAAACAAATTACAAAAATTCAAAATTTTCG
GGTTTATTACAGGGACAGCAGAGATCCAGTTTGGAAAGGACCAGCAAAGCTCCTCTGGAAAGGTGAAGGG
GCAGTAGTAATACAAGATAATAGTGACATAAAAGTAGTGCCAAGAAGAAAAGCAAAGATCATCAGGGATT
ATGGAAAACAGATGGCAGGTGATGATTGTGTGGCAAGTAGACAGGATGAGGATTAACACATGGAAAAGAT
TAGTAAAACACCATATGTATATTTCAAGGAAAGCTAAGGACTGGTTTTATAGACATCACTATGAAAGTAC
TAATCCAAAAATAAGTTCAGAAGTACACATCCCACTAGGGGATGCTAAATTAGTAATAACAACATATTGG
GGTCTGCATACAGGAGAAAGAGACTGGCATTTGGGTCAGGGAGTCTCCATAGAATGGAGGAAAAAGAGAT
ATAGCACACAAGTAGACCCTGACCTAGCAGACCAACTAATTCATCTGCACTATTTTGATTGTTTTTCAGA
ATCTGCTATAAGAAATACCATATTAGGACGTATAGTTAGTCCTAGGTGTGAATATCAAGCAGGACATAAC
AAGGTAGGATCTCTACAGTACTTGGCACTAGCAGCATTAATAAAACCAAAACAGATAAAGCCACCTTTGC
CTAGTGTTAGGAAACTGACAGAGGACAGATGGAACAAGCCCCAGAAGACCAAGGGCCACAGAGGGAGCCA
TACAATGAATGGACACTAGAGCTTTTAGAGGAACTTAAGAGTGAAGCTGTTAGACATTTTCCTAGGATAT
GGCTCCATAACTTAGGACAACATATCTATGAAACTTACGGGGATACTTGGGCAGGAGTGGAAGCCATAAT
AAGAATTCTGCAACAACTGCTGTTTATCCATTTCAGAATTGGGTGTCGACATAGCAGAATAGGCGTTACT
CGACAGAGGAGAGCAAGAAATGGAGCCAGTAGATCCTAGACTAGAGCCCTGGAAGCATCCAGGAAGTCAG
CCTAAAACTGCTTGTACCAATTGCTATTGTAAAAAGTGTTGCTTTCATTGCCAAGTTTGTTTCATGACAA
AAGCCTTAGGCATCTCCTATGGCAGGAAGAAGCGGAGACAGCGACGAAGAGCTCATCAGAACAGTCAGAC
TCATCAAGCTTCTCTATCAAAGCAGTAAGTAGTACATGTAATGCAACCTATAATAGTAGCAATAGTAGCA
TTAGTAGTAGCAATAATAATAGCAATAGTTGTGTGGTCCATAGTAATCATAGAATATAGGAAAATATTAA
GACAAAGAAAAATAGACAGGTTAATTGATAGACTAATAGAAAGAGCAGAAGACAGTGGCAATGAGAGTGA
AGGAGAAGTATCAGCACTTGTGGAGATGGGGGTGGAAATGGGGCACCATGCTCCTTGGGATATTGATGAT
CTGTAGTGCTACAGAAAAATTGTGGGTCACAGTCTATTATGGGGTACCTGTGTGGAAGGAAGCAACCACC
ACTCTATTTTGTGCATCAGATGCTAAAGCATATGATACAGAGGTACATAATGTTTGGGCCACACATGCCT
GTGTACCCACAGACCCCAACCCACAAGAAGTAGTATTGGTAAATGTGACAGAAAATTTTAACATGTGGAA
AAATGACATGGTAGAACAGATGCATGAGGATATAATCAGTTTATGGGATCAAAGCCTAAAGCCATGTGTA
AAATTAACCCCACTCTGTGTTAGTTTAAAGTGCACTGATTTGAAGAATGATACTAATACCAATAGTAGTA
GCGGGAGAATGATAATGGAGAAAGGAGAGATAAAAAACTGCTCTTTCAATATCAGCACAAGCATAAGAGA
TAAGGTGCAGAAAGAATATGCATTCTTTTATAAACTTGATATAGTACCAATAGATAATACCAGCTATAGG
TTGATAAGTTGTAACACCTCAGTCATTACACAGGCCTGTCCAAAGGTATCCTTTGAGCCAATTCCCATAC
ATTATTGTGCCCCGGCTGGTTTTGCGATTCTAAAATGTAATAATAAGACGTTCAATGGAACAGGACCATG
TACAAATGTCAGCACAGTACAATGTACACATGGAATCAGGCCAGTAGTATCAACTCAACTGCTGTTAAAT
GGCAGTCTAGCAGAAGAAGATGTAGTAATTAGATCTGCCAATTTCACAGACAATGCTAAAACCATAATAG
TACAGCTGAACACATCTGTAGAAATTAATTGTACAAGACCCAACAACAATACAAGAAAAAGTATCCGTAT
CCAGAGGGGACCAGGGAGAGCATTTGTTACAATAGGAAAAATAGGAAATATGAGACAAGCACATTGTAAC
ATTAGTAGAGCAAAATGGAATGCCACTTTAAAACAGATAGCTAGCAAATTAAGAGAACAATTTGGAAATA
ATAAAACAATAATCTTTAAGCAATCCTCAGGAGGGGACCCAGAAATTGTAACGCACAGTTTTAATTGTGG
AGGGGAATTTTTCTACTGTAATTCAACACAACTGTTTAATAGTACTTGGTTTAATAGTACTTGGAGTACT
GAAGGGTCAAATAACACTGAAGGAAGTGACACAATCACACTCCCATGCAGAATAAAACAATTTATAAACA
TGTGGCAGGAAGTAGGAAAAGCAATGTATGCCCCTCCCATCAGTGGACAAATTAGATGTTCATCAAATAT
TACTGGGCTGCTATTAACAAGAGATGGTGGTAATAACAACAATGGGTCCGAGATCTTCAGACCTGGAGGA
GGCGATATGAGGGACAATTGGAGAAGTGAATTATATAAATATAAAGTAGTAAAAATTGAACCATTAGGAG
TAGCACCCACCAAGGCAAAGAGAAGAGTGGTGCAGAGAGAAAAAAGAGCAGTGGGAATAGGAGCTTTGTT
CCTTGGGTTCTTGGGAGCAGCAGGAAGCACTATGGGCGCAGCGTCAATGACGCTGACGGTACAGGCCAGA
CAATTATTGTCTGATATAGTGCAGCAGCAGAACAATTTGCTGAGGGCTATTGAGGCGCAACAGCATCTGT
TGCAACTCACAGTCTGGGGCATCAAACAGCTCCAGGCAAGAATCCTGGCTGTGGAAAGATACCTAAAGGA
TCAACAGCTCCTGGGGATTTGGGGTTGCTCTGGAAAACTCATTTGCACCACTGCTGTGCCTTGGAATGCT
AGTTGGAGTAATAAATCTCTGGAACAGATTTGGAATAACATGACCTGGATGGAGTGGGACAGAGAAATTA
ACAATTACACAAGCTTAATACACTCCTTAATTGAAGAATCGCAAAACCAGCAAGAAAAGAATGAACAAGA
ATTATTGGAATTAGATAAATGGGCAAGTTTGTGGAATTGGTTTAACATAACAAATTGGCTGTGGTATATA
AAATTATTCATAATGATAGTAGGAGGCTTGGTAGGTTTAAGAATAGTTTTTGCTGTACTTTCTATAGTGA
ATAGAGTTAGGCAGGGATATTCACCATTATCGTTTCAGACCCACCTCCCAATCCCGAGGGGACCCGACAG
GCCCGAAGGAATAGAAGAAGAAGGTGGAGAGAGAGACAGAGACAGATCCATTCGATTAGTGAACGGATCC
TTAGCACTTATCTGGGACGATCTGCGGAGCCTGTGCCTCTTCAGCTACCACCGCTTGAGAGACTTACTCT
TGATTGTAACGAGGATTGTGGAACTTCTGGGACGCAGGGGGTGGGAAGCCCTCAAATATTGGTGGAATCT
CCTACAGTATTGGAGTCAGGAACTAAAGAATAGTGCTGTTAACTTGCTCAATGCCACAGCCATAGCAGTA
GCTGAGGGGACAGATAGGGTTATAGAAGTATTACAAGCAGCTTATAGAGCTATTCGCCACATACCTAGAA
GAATAAGACAGGGCTTGGAAAGGATTTTGCTATAAGATGGGTGGCAAGTGGTCAAAAAGTAGTGTGATTG
GATGGCCTGCTGTAAGGGAAAGAATGAGACGAGCTGAGCCAGCAGCAGATGGGGTGGGAGCAGTATCTCG
AGACCTAGAAAAACATGGAGCAATCACAAGTAGCAATACAGCAGCTAACAATGCTGCTTGTGCCTGGCTA
GAAGCACAAGAGGAGGAAGAGGTGGGTTTTCCAGTCACACCTCAGGTACCTTTAAGACCAATGACTTACA
AGGCAGCTGTAGATCTTAGCCACTTTTTAAAAGAAAAGGGGGGACTGGAAGGGCTAATTCACTCCCAAAG
AAGACAAGATATCCTTGATCTGTGGATCTACCACACACAAGGCTACTTCCCTGATTGGCAGAACTACACA
CCAGGGCCAGGGGTCAGATATCCACTGACCTTTGGATGGTGCTACAAGCTAGTACCAGTTGAGCCAGATA
AGGTAGAAGAGGCCAATAAAGGAGAGAACACCAGCTTGTTACACCCTGTGAGCCTGCATGGAATGGATGA
CCCTGAGAGAGAAGTGTTAGAGTGGAGGTTTGACAGCCGCCTAGCATTTCATCACGTGGCCCGAGAGCTG
CATCCGGAGTACTTCAAGAACTGCTGACATCGAGCTTGCTACAAGGGACTTTCCGCTGGGGACTTTCCAG
GGAGGCGTGGCCTGGGCGGGACTGGGGAGTGGCGAGCCCTCAGATGCTGCATATAAGCAGCTGCTTTTTG
CCTGTACTGGGTCTCTCTGGTTAGACCAGATCTGAGCCTGGGAGCTCTCTGGCTAACTAGGGAACCCACT
GCTTAAGCCTCAATAAAGCTTGCCTTGAGTGCTTCAAGTAGTGTGTGCCCGTCTGTTGTGTGACTCTGGT
AACTAGAGATCCCTCAGACCCTTTTAGTCAGTGTGGAAAATCTCTAGCACCCAGGAGGTAGAGGTTGCAG
TGAGCCAAGATCGCGCCACTGCATTCCAGCCTGGGCAAGAAAACAAGACTGTCTAAAATAATAATAATAA
GTTAAGGGTATTAAATATATTTATACATGGAGGTCATAAAAATATATATATTTGGGCTGGGCGCAGTGGC
TCACACCTGCGCCCGGCCCTTTGGGAGGCCGAGGCAGGTGGATCACCTGAGTTTGGGAGTTCCAGACCAG
CCTGACCAACATGGAGAAACCCCTTCTCTGTGTATTTTTAGTAGATTTTATTTTATGTGTATTTTATTCA
CAGGTATTTCTGGAAAACTGAAACTGTTTTTCCTCTACTCTGATACCACAAGAATCATCAGCACAGAGGA
AGACTTCTGTGATCAAATGTGGTGGGAGAGGGAGGTTTTCACCAGCACATGAGCAGTCAGTTCTGCCGCA
GACTCGGCGGGTGTCCTTCGGTTCAGTTCCAACACCGCCTGCCTGGAGAGAGGTCAGACCACAGGGTGAG
GGCTCAGTCCCCAAGACATAAACACCCAAGACATAAACACCCAACAGGTCCACCCCGCCTGCTGCCCAGG
CAGAGCCGATTCACCAAGACGGGAATTAGGATAGAGAAAGAGTAAGTCACACAGAGCCGGCTGTGCGGGA
GAACGGAGTTCTATTATGACTCAAATCAGTCTCCCCAAGCATTCGGGGATCAGAGTTTTTAAGGATAACT
TAGTGTGTAGGGGGCCAGTGAGTTGGAGATGAAAGCGTAGGGAGTCGAAGGTGTCCTTTTGCGCCGAGTC
AGTTCCTGGGTGGGGGCCACAAGATCGGATGAGCCAGTTTATCAATCCGGGGGTGCCAGCTGATCCATGG
AGTGCAGGGTCTGCAAAATATCTCAAGCACTGATTGATCTTAGGTTTTACAATAGTGATGTTACCCCAGG
AACAATTTGGGGAAGGTCAGAATCTTGTAGCCTGTAGCTGCATGACTCCTAAACCATAATTTCTTTTTTG
TTTTTTTTTTTTTATTTTTGAGACAGGGTCTCACTCTGTCACCTAGGCTGGAGTGCAGTGGTGCAATCAC
AGCTCACTGCAGCCTCAACGTCGTAAGCTCAAGCGATCCTCCCACCTCAGCCTGCCTGGTAGCTGAGACT
ACAAGCGACGCCCCAGTTAATTTTTGTATTTTTGGTAGAGGCAGCGTTTTGCCGTGTGGCCCTGGCTGGT
CTCGAACTCCTGGGCTCAAGTGATCCAGCCTCAGCCTCCCAAAGTGCTGGGACAACCGGGGCCAGTCACT
GCACCTGGCCCTAAACCATAATTTCTAATCTTTTGGCTAATTTGTTAGTCCTACAAAGGCAGTCTAGTCC
CCAGGCAAAAAGGGGGTTTGTTTCGGGAAAGGGCTGTTACTGTCTTTGTTTCAAACTATAAACTAAGTTC
CTCCTAAACTTAGTTCGGCCTACACCCAGGAATGAACAAGGAGAGCTTGGAGGTTAGAAGCACGATGGAA
TTGGTTAGGTCAGATCTCTTTCACTGTCTGAGTTATAATTTTGCAATGGTGGTTCAAAGACTGCCCGCTT
CTGACACCAGTCGCTGCATTAATGAATCGGCCAACGCGCGGGGAGAGGCGGTTTGCGTATTGGGCGCTCT
TCCGCTTCCTCGCTCACTGACTCGCTGCGCTCGGTCGTTCGGCTGCGGCGAGCGGTATCAGCTCACTCAA
AGGCGGTAATACGGTTATCCACAGAATCAGGGGATAACGCAGGAAAGAACATGTGAGCAAAAGGCCAGCA
AAAGGCCAGGAACCGTAAAAAGGCCGCGTTGCTGGCGTTTTTCCATAGGCTCCGCCCCCCTGACGAGCAT
CACAAAAATCGACGCTCAAGTCAGAGGTGGCGAAACCCGACAGGACTATAAAGATACCAGGCGTTTCCCC
CTGGAAGCTCCCTCGTGCGCTCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTATCGTCTTG
AGTCCAACCCGGTAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGAAGAACAGTATTT
GGTATCTGCGCTCTGCTGAAGCCAGTTACCTTCGGAAAAAGAGTTGGTAGCTCTTGATCCGGCAAACAAA
CCACCGCTGGTAGCGGTGGTTTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACTCACGTTAAGGGATTTTGGTC
ATGAGATTATCAAAAAGGATCTTCACCTAGATCCTTTTAAATTAAAAATGAAGTTTTAAATCAATCTAAA
GTATATATGAGTAAACTTGGTCTGACAGTTACCAATGCTTAATCAGTGAGGCACCTATCTCAGCGATCTG
TCTATTTCGTTCATCCATAGTTGCCTGACTCCCCGTCGTGTAGATAACTACGATACGGGAGGGCTTACCA
TCTGGCCCCAGTGCTGCAATGATACCGCGAGACCCACGCTCACCGGCTCCAGATTTATCAGCAATAAACC
AGCCAGCCGGAAGGGCCGAGCGCAGAAGTGGTCCTGCAACTTTATCCGCCTCCATCCAGTCTATTAATTG
TTGCCGGGAAGCTAGAGTAAGTAGTTCGCCAGTTAATAGTTTGCGCAACGTTGTTGCCATTGCTACAGGC
ATCGTGGTGTCACGCTCGTCGTTTGGTATGGCTTCATTCAGCTCCGGTTCCCAACGATCAAGGCGAGTTA
CATGATCCCCCATGTTGTGCAAAAAAGCGGTTAGCTCCTTCGGTCCTCCGATCGTTGTCAGAAGTAAGTT
GGCCGCAGTGTTATCACTCATGGTTATGGCAGCACTGCATAATTCTCTTACTGTCATGCCATCCGTAAGA
TGCTTTTCTGTGACTGGTGAGTACTCAACCAAGTCATTCTGAGAATAGTGTATGCGGCGACCGAGTTGCT
CTTGCCCGGCGTCAATACGGGATAATACCGCGCCACATAGCAGAACTTTAAAAGTGCTCATCATTGGAAA
ACGTTCTTCGGGGCGAAAACTCTCAAGGATCTTACCGCTGTTGAGATCCAGTTCGATGTAACCCACTCGT
GCACCCAACTGATCTTCAGCATCTTTTACTTTCACCAGCGTTTCTGGGTGAGCAAAAACAGGAAGGCAAA
ATGCCGCAAAAAAGGGAATAAGGGCGACACGGAAATGTTGAATACTCATACTCTTCCTTTTTCAATATTA
TTGAAGCATTTATCAGGGTTATTGTCTCATGAGCGGATACATATTTGAATGTATTTAGAAAAATAAACAA
ATAGGGGTTCCGCGCACATTTCCCCGAAAAGTGCCACCTGACGTCTAAGAAACCATTATTATCATGACAT
TAACCTATAAAAATAGGCGTATCACGAGGCCCTTTCGTCTCGCGCGTTTCGGTGATGACGGTGAAAACCT
CTGACACATGCAGCTCCCGGAGACGGTCACAGCTTGTCTGTAAGCGGATGCCGGGAGCAGACAAGCCCGT
CAGGGCGCGTCAGCGGGTGTTGGCGGGTGTCGGGGCTGGCTTAACTATGCGGCATCAGAGCAGATTGTAC
TGAGAGTGCACCATATGCGGTGTGAAATACCGCACAGATGCGTAAGGAGAAAATACCGCATCAGGCGCCA
TTCGCCATTCAGGCTGCGCAACTGTTGGGAAGGGCGATCGGTGCGGGCCTCTTCGCTATTACGCCAGGGG
AGGCAGAGATTGCAGTAAGCTGAGATCGCAGCACTGCACTCCAGCCTGGGCGACAGAGTAAGACTCTGTC
TCAAAAATAAAATAAATAAATCAATCAGATATTCCAATCTTTTCCTTTATTTATTTATTTATTTTCTATT
TTGGAAACACAGTCCTTCCTTATTCCAGAATTACACATATATTCTATTTTTCTTTATATGCTCCAGTTTT
TTTTAGACCTTCACCTGAAATGTGTGTATACAAAATCTAGGCCAGTCCAGCAGAGCCTAAAGGTAAAAAA
TAAAATAATAAAAAATAAATAAAATCTAGCTCACTCCTTCACATCAAAATGGAGATACAGCTGTTAGCAT
TAAATACCAAATAACCCATCTTGTCCTCAATAATTTTAAGCGCCTCTCTCCACCACATCTAACTCCTGTC
AAAGGCATGTGCCCCTTCCGGGCGCTCTGCTGTGCTGCCAACCAACTGGCATGTGGACTCTGCAGGGTCC
CTAACTGCCAAGCCCCACAGTGTGCCCTGAGGCTGCCCCTTCCTTCTAGCGGCTGCCCCCACTCGGCTTT
GCTTTCCCTAGTTTCAGTTACTTGCGTTCAGCCAAGGTCTGAAACTAGGTGCGCACAGAGCGGTAAGACT
GCGAGAGAAAGAGACCAGCTTTACAGGGGGTTTATCACAGTGCACCCTGACAGTCGTCAGCCTCACAGGG
GGTTTATCACATTGCACCCTGACAGTCGTCAGCCTCACAGGGGGTTTATCACAGTGCACCCTTACAATCA
TTCCATTTGATTCACAATTTTTTTAGTCTCTACTGTGCCTAACTTGTAAGTTAAATTTGATCAGAGGTGT
GTTCCCAGAGGGGAAAACAGTATATACAGGGTTCAGTACTATCGCATTTCAGGCCTCCACCTGGGTCTTG
GAATGTGTCCCCCGAGGGGTGATGACTACCTCAGTTGGATCTCCACAGGTCACAGTGACACAAGATAACC
AAGACACCTCCCAAGGCTACCACAATGGGCCGCCCTCCACGTGCACATGGCCGGAGGAACTGCCATGTCG
GAGGTGCAAGCACACCTGCGCATCAGAGTCCTTGGTGTGGAGGGAGGGACCAGCGCAGCTTCCAGCCATC
CACCTGATGAACAGAACCTAGGGAAAGCCCCAGTTCTACTTACACCAGGAAAGGC
SEQ ID NO. 2
Amino Acid
Vif protein (wild-type)
HIV-1
MENRWQVMIVWQVDRMRINTWKRLVKHHMYISRKAKDWFYRHHYESTNPKISSEVHIPLGDAKLVITTYWGLHTGER
DWHLGQGVSIEWRKKRYSTQVDPDLADQLIHLHYFDCFSESAIRNTILGRIVSPRCEYQAGHNKVGSLQYLALAALI
KPKQIKPPLPSVRKLTEDRWNKPQKTKGHRGSHTMNGH
SEQ ID NO. 3**
Amino Acid
Capsid Peptide (wild-type)
HIV-1
PIVQNLQGQMVHQAISPRTLNAWVKVVEEKAFSPEVIPMFSALSEGATPQDLNTMLNTVGGHQAAMQMLKETINEEA
AEWDRLHPVHAGPIAPGQMREPRGSDIAGTTSTLQEQIGWMTHNPPIPVGEIYKRWIILGLNKIVRMYSP
SEQ ID NO. 4
Amino Acid
Tat (wild-type)
HIV-1
MEPVDPRLEPWKHPGSQPKTACTNCYCKKCCFHCQVCFMTKALGISYGRKKRRQRRRAHQNSQTHQASLSKQPTSQS
RGDPTGPKE
SEQ ID NO. 5
Amino Acid
Env (wild-type)
HIV-1
MRVKEKYQHLWRWGWKWGTMLLGILMICSATEKLWVTVYYGVPVWKEATTTLFCASDAKAYDTEVHNVWATHACVPT
DPNPQEVVLVNVTENFNMWKNDMVEQMHEDIISLWDQSLKPCVKLTPLCVSLKCTDLKNDTNTNSSSGRMIMEKGEI
KNCSFNISTSIRDKVQKEYAFFYKLDIVPIDNTSYRLISCNTSVITQACPKVSFEPIPIHYCAPAGFAILKCNNKTF
NGTGPCTNVSTVQCTHGIRPVVSTQLLLNGSLAEEDVVIRSANFTDNAKTIIVQLNTSVEINCTRPNNNTRKSIRIQ
RGPGRAFVTIGKIGNMRQAHCNISRAKWNATLKQIASKLREQFGNNKTIIFKQSSGGDPEIVTHSFNCGGEFFYCNS
TQLFNSTWFNSTWSTEGSNNTEGSDTITLPCRIKQFINMWQEVGKAMYAPPISGQIRCSSNITGLLLTRDGGNNNNG
SEIFRPGGGDMRDNWRSELYKYKVVKIEPLGVAPTKAKRRVVQREKRAVGIGALFLGFLGAAGSTMGAASMTLTVQA
RQLLSDIVQQQNNLLRAIEAQQHLLQLTVWGIKQLQARILAVERYLKDQQLLGIWGCSGKLICTTAVPWNASWSNKS
LEQIWNNMTWMEWDREINNYTSLIHSLIEESQNQQEKNEQELLELDKWASLWNWFNITNWLWYIKLFIMIVGGLVGL
RIVFAVLSIVNRVRQGYSPLSFQTHLPIPRGPDRPEGIEEEGGERDRDRSIRLVNGSLALIWDDLRSLCLFSYHRLR
DLLLIVTRIVELLGRRGWEALKYWWNLLQYWSQELKNSAVNLLNATAIAVAEGTDRVIEVLQAAYRAIRHIPRRIRQ
GLERILL
SEQ ID NO. 6
DNA
Vif (SIVmac)
Rhesus macaque
ATGGAGGAGGAAAAGAGGTGGATAGCAGTTCCCACATGGAGGATACCGGAGAGGCTAGAGAGGTGGCATAGCCTCAT
AAAATATCTGAAATATAAAACTAAAGATCTACAAAAGGTTTGCTATGTGCCCCATTTTAAGGTCGGATGGGCATGGT
GGACCTGCAGCAGAGTAATCTTCCCCCTACAGGAAGGAAGCCATTTAGAAGTACAAGGGTATTGGCATTTGACACCA
GAAAGAGGGTGGCTCAGTACTTATGCAGTGAGGATAACCTGGTACTCAAGGAACTTTTGGACAGATGTAACACCAGA
CTATGCAGACATTTTACTGCATAGCACTTATTTCCCTTGCTTTACAGCGGGAGAAGTGAGAAGGGCCATCAGGGGAG
AACAACTGCTGTCTTGCTGCAAGTTCCCGAGAGCTCATAGGTACCAGGTACCAAGCCTACAGTACTTAGCACTAAAA
GTAGTAAGCGACGTCAGATCCCAGGGAGAGAATCCCACCTGGAAACAGTGGAGAAGAGACAATAGGAGAGGCCTTCG
AATGGCTAAACAGAACAGTAGAGGAGATAAACAGAGAGGCAGTAAACCACCTACCAAGGGAGCTGATTTTCCAGGTT
TGGCAAAGGTCTTGGGAATACTGGCATGA
SEQ ID NO. 7
DNA
Vif (SIVptm)
Pig-tail macaque
ATGGAGGAGGAAAAGAGGTGGATAGCAGTTCCCACATGGAGGATACCGGAGAGGCTAGAGAGGTGGCATAGCCTCAT
AAAATATCTGAAATATAAAACTAAAGATCTACAAAAGGTTTGCTATGTGCCCCATCATAAGGTCGGATGGGCATGGT
GGACCTGCAGCAGAGTAATCTTCCCACTACAAGAAGAAAGCCAGTTAGAAGTACAAGGGTATTGGAATTTAACACCA
GAAAGAGGGTGGCTCAGTACTTATGCAGTGAGAATAACCTGGTACTCAAGGAACTTTTGGACAGATGTAACACCAGA
CTATGCAGACATTTTACTGCATAGCACTTATTTCCCTTGCTTTACAGCGGGAGAAGTGAGAAGGGCCATCAGGGGAG
AACAACTGCTGTCTTGCTGCAGGTTCCCGAGAGCTCATAAGAACCAGGTACCAAGTCTACAGTACTTAGCACTGAGA
GTAGTAAGTTACGTCAGATCCCAGAGAGAGAATCCCACCTGGAAACAGTGGAGAAGAGACAATAGGAGAAGCCTTCG
AATGGCTAAACAGAACAGTAGAGGAGATAAACAGAGAGGCAGTAAACCACCTACCAAGGGAGCTGATTTTCCAGGTT
TGGCAAAGGTCTTGGGAATACTGGCATGA
SEQ ID NO. 8
Amino Acid
Vif protein (wild type)
HIV-1
MENRWQVMIVWQVDRMRINTWKRLVKHHMYISRKAKDWFYRHHYESTNPKISSEVHIPLGDAKLVITTYWGLHTGER
DWHLGQGVSIEWRKKRYSTQVDPDLADQLIHLHYFDCFSESAIRNTILGRIVSPRCEYQAGHNKVGSLQYLALAALI
KPKQIKPPLPSVRKLTEDRWNKPQKTKGHRGSHTMNGH
SEQ ID NO. 9*
Amino Acid
Capsid Peptide (A-Mutant)
HIV-1
PIVQNLQGQMVHQAISPRTLNAWVKVVEEKAFSPEVIPMFSALSEGATPQDLNTMLNTVGGHQAAMQMLKETINEEA
AEWDRLH PVPGPIPAGQMREPRGSDIAGTTSTLQEQIGWMTHNPPIPVGEIYKRWIILGLNKIVRMYSP
SEQ ID NO. 10* **
Amino Acid
Capsid Peptide (H120R)
HIV-1
PIVQNLQGQMVHQAISPRTLNAWVKVVEEKAFSPEVIPMFSALSEGATPQDLNTMLNTVGGHQAAMQMLKETINEEA
AEWDRLHPVPGPIPAGQMREPRGSDIAGTTSTLQEQIGWMTRNPPIPVGEIYKRWIILGLNKIVRMYSP
SEQ ID NO. 11*
Amino Acid
Capsid Peptide (A-Mutant H120R)
HIV-1
PIVQNLQGQMVHQAISPRTLNAWVKVVEEKAFSPEVIPMFSALSEGATPQDLNTMLNTVGGHQAAMQMLKETINEEA
AEWDRLHPVPGPIPAGQMREPRGSDIAGTTSTLQEQIGWMTRNPPIPVGEIYKRWIILGLNKIVRMYSP
SEQ ID NO. 12*
Amino Acid
Tat (A58T)
HIV-1
MEPVDPRLEPWKHPGSQPKTACTNCYCKKCCFHCQVCFMTKALGISYGRKKRRQRRRTHWNSQTHQASLSKWPTSQS
RGDPTGPKE
SEQ ID NO. 13*
Amino Acid
Env (H9R)
HIV-1
MRVKEKYQRLWRWGWKWGTMLLGILMICSATEKLWVTVYYGVPVWKEATTTLFCASDAKAYDTEVHNVWATHACVPT
DPNPQEVVLVNVTENFNMWKNDMVEQMHEDIISLWDQSLKPCVKLTPLCVSLKCTDLKNDTNTNSSSGRMIMEKGEI
KNCSFNISTSIRDKVQKEYAFFYKLDIVPIDNTSYRLISCNTSVITQACPKVSFEPIPIHYCAPAGFAILKCNNKTF
NGTGPCTNVSTVQCTHGIRPVVSTQLLINGSLAEEDVVIRSANFTDNAKTIIVQLNTSVEINCTRPNNNTRKSIRIQ
RGPGRAFVTIGKIGNMRQAHCNISRAKWNATLKQIASKLREQFGNNKTIIFKQSSGGDPEIVTHSFNCGGEFFYCNS
TQLFNSTWFNSTWSTEGSNNTEGSDTITLPCRIKQFINMWQEVGKAMYAPPISGQIRCSSNITGLLLTRDGGNNNNG
SEIFRPGGGDMRDNWRSELYKYKVVKIEPLGVAPTKAKRRVVQREKRAVGIGALFLGFLGAAGSTMGAASMTLTVQA
RQLLSDIVQQQNNLLRAIEAQQHLLQLTVWGIKQLQARILAVERYLKDQQLLGIWGCSGKLICTTAVPWNASWSNKS
LEQIWNNMTWMEWDREINNYTSLIHSLIEESQNQQEKNEQELLELDKWASLWNWFNITNWLWYIKLFIMIVGGLVGL
RIVFAVLSIVNRVRQGYSPLSFQTHLPIPRGPDRPEGIEEEGGERDRDRSIRLVNGSLALIWDDLRSLCLFSYHRLR
DLLLIVTRIVELLGRRGWEALKYWWNLLQYWSQELKNSAVNLLNATAIAVAEGTDRVIEVLQAAYRAIRHIPRRIRQ
GLERILL
SEQ ID NO. 14*
Amino Acid
Env (L10W)
HIV-1
MRVKEKYQHWWRWGWKWGTMLLGILMICSATEKLWVTVYYGVPVWKEATTTLFCASDAKAYDTEVHNVWATHACVPT
DPNPQEVVLVNVTENFNMWKNDMVEQMHEDIISLWDQSLKPCVKLTPLCVSLKCTDLKNDTNTNSSSGRMIMEKGEI
KNCSFNISTSIRDKVQKEYAFFYKLDIVPIDNTSYRLISCNTSVITQACPKVSFEPIPIHYCAPAGFAILKCNNKTF
NGTGPCTNVSTVQCTHGIRPVVSTQLLINGSLAEEDVVIRSANFTDNAKTIIVQLNTSVEINCTRPNNNTRKSIRIQ
RGPGRAFVTIGKIGNMRQAHCNISRAKWNATLKQIASKLREQFGNNKTIIFKQSSGGDPEIVTHSFNCGGEFFYCNS
TQLFNSTWFNSTWSTEGSNNTEGSDTITLPCRIKQFINMWQEVGKAMYAPPISGQIRCSSNITGLLLTRDGGNNNNG
SEIFRPGGGDMRDNWRSELYKYKVVKIEPLGVAPTKAKRRVVQREKRAVGIGALFLGFLGAAGSTMGAASMTLTVQA
RQLLSDIVQQQNNLLRAIEAQQHLLQLTVWGIKQLQARILAVERYLKDQQLLGIWGCSGKLICTTAVPWNASWSNKS
LEQIWNNMTWMEWDREINNYTSLIHSLIEESQNQQEKNEQELLELDKWASLWNWFNITNWLWYIKLFIMIVGGLVGL
RIVFAVLSIVNRVRQGYSPLSFQTHLPIPRGPDRPEGIEEEGGERDRDRSIRLVNGSLALIWDDLRSLCLFSYHRLR
DLLLIVTRIVELLGRRGWEALKYWWNLLQYWSQELKNSAVNLLNATAIAVAEGTDRVIEVLQAAYRAIRHIPRRIRQ
GLERILL
SEQ ID NO. 15*
Amino Acid
Env (D545G)
HIV-1
MRVKEKYQHLWRWGWKWGTMLLGILMICSATEKLWVTVYYGVPVWKEATTTLFCASDAKAYDTEVHNVWATHACVPT
DPNPQEVVLVNVTENFNMWKNDMVEQMHEDIISLWDQSLKPCVKLTPLCVSLKCTDLKNDTNTNSSSGRMIMEKGEI
KNCSFNISTSIRDKVQKEYAFFYKLDIVPIDNTSYRLISCNTSVITQACPKVSFEPIPIHYCAPAGFAILKCNNKTF
NGTGPCTNVSTVQCTHGIRPVVSTQLLINGSLAEEDVVIRSANFTDNAKTIIVQLNTSVEINCTRPNNNTRKSIRIQ
RGPGRAFVTIGKIGNMRQAHCNISRAKWNATLKQIASKLREQFGNNKTIIFKQSSGGDPEIVTHSFNCGGEFFYCNS
TQLFNSTWFNSTWSTEGSNNTEGSDTITLPCRIKQFINMWQEVGKAMYAPPISGQIRCSSNITGLLLTRDGGNNNNG
SEIFRPGGGDMRDNWRSELYKYKVVKIEPLGVAPTKAKRRVVQREKRAVGIGALFLGFLGAAGSTMGAASMTLTVQA
RQLLSGIVQQQNNLLRAIEAQQHLLQLTVWGIKQLQARILAVERYLKDQQLLGIWGCSGKLICTTAVPWNASWSNKS
LEQIWNNMTWMEWDREINNYTSLIHSLIEESQNQQEKNEQELLELDKWASLWNWFNITNWLWYIKLFIMIVGGLVGL
RIVFAVLSIVNRVRQGYSPLSFQTHLPIPRGPDRPEGIEEEGGERDRDRSIRLVNGSLALIWDDLRSLCLFSYHRLR
DLLLIVTRIVELLGRRGWEALKYWWNLLQYWSQELKNSAVNLLNATAIAVAEGTDRVIEVLQAAYRAIRHIPRRIRQ
GLERILL
SEQ ID NO. 16*
Amino Acid
Env (D545G, L10W)
HIV-1
MRVKEKYQHWWRWGWKWGTMLLGILMICSATEKLWVTVYYGVPVWKEATTTLFCASDAKAYDTEVHNVWATHACVPT
DPNPQEVVLVNVTENFNMWKNDMVEQMHEDIISLWDQSLKPCVKLTPLCVSLKCTDLKNDTNTNSSSGRMIMEKGEI
KNCSFNISTSIRDKVQKEYAFFYKLDIVPIDNTSYRLISCNTSVITQACPKVSFEPIPIHYCAPAGFAILKCNNKTF
NGTGPCTNVSTVQCTHGIRPVVSTQLLINGSLAEEDVVIRSANFTDNAKTIIVQLNTSVEINCTRPNNNTRKSIRIQ
RGPGRAFVTIGKIGNMRQAHCNISRAKWNATLKQIASKLREQFGNNKTIIFKQSSGGDPEIVTHSFNCGGEFFYCNS
TQLFNSTWFNSTWSTEGSNNTEGSDTITLPCRIKQFINMWQEVGKAMYAPPISGQIRCSSNITGLLLTRDGGNNNNG
SEIFRPGGGDMRDNWRSELYKYKVVKIEPLGVAPTKAKRRVVQREKRAVGIGALFLGFLGAAGSTMGAASMTLTVQA
RQLLSGIVQQQNNLLRAIEAQQHLLQLTVWGIKQLQARILAVERYLKDQQLLGIWGCSGKLICTTAVPWNASWSNKS
LEQIWNNMTWMEWDREINNYTSLIHSLIEESQNQQEKNEQELLELDKWASLWNWFNITNWLWYIKLFIMIVGGLVGL
RIVFAVLSIVNRVRQGYSPLSFQTHLPIPRGPDRPEGIEEEGGERDRDRSIRLVNGSLALIWDDLRSLCLFSYHRLR
DLLLIVTRIVELLGRRGWEALKYWWNLLQYWSQELKNSAVNLLNATAIAVAEGTDRVIEVLQAAYRAIRHIPRRIRQ
GLERILL
SEQ ID NO. 17*
Amino Acid
Env (D545G, H9R)
HIV-1
MRVKEKYQRLWRWGWKWGTMLLGILMICSATEKLWVTVYYGVPVWKEATTTLFCASDAKAYDTEVHNVWATHACVPT
DPNPQEVVLVNVTENFNMWKNDMVEQMHEDIISLWDQSLKPCVKLTPLCVSLKCTDLKNDTNTNSSSGRMIMEKGEI
KNCSFNISTSIRDKVQKEYAFFYKLDIVPIDNTSYRLISCNTSVITQACPKVSFEPIPIHYCAPAGFAILKCNNKTF
NGTGPCTNVSTVQCTHGIRPVVSTQLLINGSLAEEDVVIRSANFTDNAKTIIVQLNTSVEINCTRPNNNTRKSIRIQ
RGPGRAFVTIGKIGNMRQAHCNISRAKWNATLKQIASKLREQFGNNKTIIFKQSSGGDPEIVTHSENCGGEFFYCNS
TQLFNSTWFNSTWSTEGSNNTEGSDTITLPCRIKQFINMWQEVGKAMYAPPISGQIRCSSNITGLLLTRDGGNNNNG
SEIFRPGGGDMRDNWRSELYKYKVVKIEPLGVAPTKAKRRVVQREKRAVGIGALFLGFLGAAGSTMGAASMTLTVQA
RQLLSGIVQQQNNLLRAIEAQQHLLQLTVWGIKQLQARILAVERYLKDQQLLGIWGCSGKLICTTAVPWNASWSNKS
LEQIWNNMTWMEWDREINNYTSLIHSLIEESQNQQEKNEQELLELDKWASLWNWFNITNWLWYIKLFIMIVGGLVGL
RIVFAVLSIVNRVRQGYSPLSFQTHLPIPRGPDRPEGIEEEGGERDRDRSIRLVNGSLALIWDDLRSLCLFSYHRLR
DLLLIVTRIVELLGRRGWEALKYWWNLLQYWSQELKNSAVNLLNATAIAVAEGTDRVIEVLQAAYRAIRHIPRRIRQ
GLERILL
SEQ ID NO. 18*
Amino Acid
Env (D545G, H9R, L10W)
HIV-1
MRVKEKYQRWWRWGWKWGTMLLGILMICSATEKLWVTVYYGVPVWKEATTTLFCASDAKAYDTEVHNVWATHACVPT
DPNPQEVVLVNVTENFNMWKNDMVEQMHEDIISLWDQSLKPCVKLTPLCVSLKCTDLKNDTNTNSSSGRMIMEKGEI
KNCSFNISTSIRDKVQKEYAFFYKLDIVPIDNTSYRLISCNTSVITQACPKVSFEPIPIHYCAPAGFAILKCNNKTF
NGTGPCTNVSTVQCTHGIRPVVSTQLLLNGSLAEEDVVIRSANFTDNAKTIIVQLNTSVEINCTRPNNNTRKSIRIQ
RGPGRAFVTIGKIGNMRQAHCNISRAKWNATLKQIASKLREQFGNNKTIIFKQSSGGDPEIVTHSFNCGGEFFYCNS
TQLFNSTWFNSTWSTEGSNNTEGSDTITLPCRIKQFINMWQEVGKAMYAPPISGQIRCSSNITGLLLTRDGGNNNNG
SEIFRPGGGDMRDNWRSELYKYKVVKIEPLGVAPTKAKRRVVQREKRAVGIGALFLGFLGAAGSTMGAASMTLTVQA
RQLLSGIVQQQNNLLRAIEAQQHLLQLTVWGIKQLQARILAVERYLKDQQLLGIWGCSGKLICTTAVPWNASWSNKS
LEQIWNNMTWMEWDREINNYTSLIHSLIEESQNQQEKNEQELLELDKWASLWNWENITNWLWYIKLFIMIVGGLVGL
RIVFAVLSIVNRVRQGYSPLSFQTHLPIPRGPDRPEGIEEEGGERDRDRSIRLVNGSLALIWDDLRSLCLFSYHRLR
DLLLIVTRIVELLGRRGWEALKYWWNLLQYWSQELKNSAVNLLNATAIAVAEGTDRVIEVLQAAYRAIRHIPRRIRQ
GLERILL
SEQ ID NO. 19*
Amino Acid
Env (D167G)
HIV-1
MRVKEKYQHLWRWGWKWGTMLLGILMICSATEKLWVTVYYGVPVWKEATTTLFCASDAKAYDTEVHNVWATHACVPT
DPNPQEVVLVNVTENFNMWKNDMVEQMHEDIISLWDQSLKPCVKLTPLCVSLKCTDLKNDTNTNSSSGRMIMEKGEI
KNCSFNISTSIRGKVQKEYAFFYKLDIVPIDNTSYRLISCNTSVITQACPKVSFEPIPIHYCAPAGFAILKCNNKTF
NGTGPCTNVSTVQCTHGIRPVVSTQLLINGSLAEEDVVIRSANFTDNAKTIIVQLNTSVEINCTRPNNNTRKSIRIQ
RGPGRAFVTIGKIGNMRQAHCNISRAKWNATLKQIASKLREQFGNNKTIIFKQSSGGDPEIVTHSFNCGGEFFYCNS
TQLFNSTWFNSTWSTEGSNNTEGSDTITLPCRIKQFINMWQEVGKAMYAPPISGQIRCSSNITGLLLTRDGGNNNNG
SEIFRPGGGDMRDNWRSELYKYKVVKIEPLGVAPTKAKRRVVQREKRAVGIGALFLGFLGAAGSTMGAASMTLTVQA
RQLLSDIVQQQNNLLRAIEAQQHLLQLTVWGIKQLQARILAVERYLKDQQLLGIWGCSGKLICTTAVPWNASWSNKS
LEQIWNNMTWMEWDREINNYTSLIHSLIEESQNQQEKNEQELLELDKWASLWNWFNITNWLWYIKLFIMIVGGLVGL
RIVFAVLSIVNRVRQGYSPLSFQTHLPIPRGPDRPEGIEEEGGERDRDRSIRLVNGSLALIWDDLRSLCLFSYHRLR
DLLLIVTRIVELLGRRGWEALKYWWNLLQYWSQELKNSAVNLLNATAIAVAEGTDRVIEVLQAAYRAIRHIPRRIRQ
GLERILL
SEQ ID NO. 20*
Amino Acid
Env (D545G, H9R, L10W, D167G)
HIV-1
MRVKEKYQRWWRWGWKWGTMLLGILMICSATEKLWVTVYYGVPVWKEATTTLFCASDAKAYDTEVHNVWATHACVPT
DPNPQEVVLVNVTENFNMWKNDMVEQMHEDIISLWDQSLKPCVKLTPLCVSLKCTDLKNDTNTNSSSGRMIMEKGEI
KNCSFNISTSIRGKVQKEYAFFYKLDIVPIDNTSYRLISCNTSVITQACPKVSFEPIPIHYCAPAGFAILKCNNKTF
NGTGPCTNVSTVQCTHGIRPVVSTQLLINGSLAEEDVVIRSANFTDNAKTIIVQLNTSVEINCTRPNNNTRKSIRIQ
RGPGRAFVTIGKIGNMRQAHCNISRAKWNATLKQIASKLREQFGNNKTIIFKQSSGGDPEIVTHSFNCGGEFFYCNS
TQLFNSTWFNSTWSTEGSNNTEGSDTITLPCRIKQFINMWQEVGKAMYAPPISGQIRCSSNITGLLLTRDGGNNNNG
SEIFRPGGGDMRDNWRSELYKYKVVKIEPLGVAPTKAKRRVVQREKRAVGIGALFLGFLGAAGSTMGAASMTLTVQA
RQLLSGIVQQQNNLLRAIEAQQHLLQLTVWGIKQLQARILAVERYLKDQQLLGIWGCSGKLICTTAVPWNASWSNKS
LEQIWNNMTWMEWDREINNYTSLIHSLIEESQNQQEKNEQELLELDKWASLWNWFNITNWLWYIKLFIMIVGGLVGL
RIVEAVLSIVNRVRQGYSPLSFQTHLPIPRGPDRPEGIEEEGGERDRDRSIRLVNGSLALIWDDLRSLCLFSYHRLR
DLLLIVTRIVELLGRRGWEALKYWWNLLQYWSQELKNSAVNLLNATAIAVAEGTDRVIEVLQAAYRAIRHIPRRIRQ
GLERILL
*Mutations are shown in BQLD UNDERLINED

1-133. (canceled)

134. A HIV-1 variant identified by ATCC patent deposit number PTA-127573.

135. The HIV-1 variant of claim 134, wherein said HIV-1 variant is isolated.

136. The HIV-1 variant of claim 134, wherein a cell is infected with the HIV-1 variant.

137. The HIV-1 variant of claim 136, wherein said cell comprises a mammalian cell.

138. The HIV-1 variant of claim 137, wherein said mammalian cell comprises an Aotus nancymaae cell.

139. The HIV-1 variant of claim 134, wherein an animal is infected with the HIV-1 variant.

140. The HIV-1 variant of claim 139, wherein said animal comprises a mammal.

141. The HIV-1 variant of claim 140, wherein said mammal comprises Aotus nancymaae.

142. A pharmaceutical composition comprising the HIV-1 variant of claim 134, and a pharmaceutically acceptable carrier.

143. A HIV-1 variant genetically modified to infect Aotus nancymaae, wherein said HIV-1 variant is selected from:
HIV-1 variant identified by ATCC patent deposit number PTA-127572;
HIV-1 variant identified by ATCC patent deposit number PTA-127573;
HIV-1 variant identified by ATCC patent deposit number PTA-127574; and
HIV-1 variant identified by ATCC patent deposit number PTA-127575.

144. The HIV-1 variant of claim 143, wherein said HIV-1 variant is isolated.

145. The HIV-1 variant of claim 143, wherein a cell is infected with the HIV-1 variant.

146. The HIV-1 variant of claim 145, wherein said cell comprises a mammalian cell.

147. The HIV-1 variant of claim 146, wherein said mammalian cell comprises an Aotus nancymaae cell.

148. The HIV-1 variant of claim 143, wherein an animal infected with the HIV-1 variant.

149. The HIV-1 variant of claim 148, wherein said animal comprises a mammal.

150. The HIV-1 variant of claim 149, wherein said mammal comprises Aotus nancymaae.

151. A pharmaceutical composition comprising the HIV-1 variant of claim 143, and a pharmaceutically acceptable carrier.

152. An Aotus nancymaae animal infected with the HIV-1 variant of claim 143.
